(19) United States
(12) Patent Application Publication
(10) Pub. No.: US 2011/0312945 A1
(43) Pub. Date: Dec. 22, 2011

(54) CRTH2 MODULATORS

(76) Inventors: James Jia, Somerville, MA (US); Ara Mermerian, Melrose, MA (US); Charles Kim, Cambridge, MA (US); Regina Graul, Duxbury, MA (US); Joel Moore, Lexington, MA (US)

(21) Appl. No.: 13/121,669

(22) PCT Filed: Oct. 1, 2009
(86) PCT No.: PCT/US2009/059265

§ 371 Date: Aug. 9, 2011

Related U.S. Application Data
(60) Provisional application No. 61/101,777, filed on Oct. 1, 2008.
(60) Provisional application No. 61/220,755, filed on Jun. 26, 2009.

(51) Int. Cl. A61K 31/55; C07D 209/12; C07D 403/12; C07D 401/12; C07D 413/14; C07D 409/12; A61K 31/5377; A61K 31/403; A61K 31/496; A61K 31/454; A61P 11/06; A61P 29/00; A61P 37/08; A61P 11/00; A61P 11/02; A61P 19/02; A61P 19/08; A61P 17/06; A61P 17/02; A61P 3/10; A61P 1/00; C07D 413/12

(52) U.S. Cl. 514/217.08; 544/143; 548/516; 544/373; 546/201; 540/602; 514/235.2; 514/412; 514/254.09; 514/323; 514/414

(57) ABSTRACT

Modulators of CRTH2, particularly antagonists of CRTH2, that are useful for treating various disorders, including asthma and respiratory disorders are disclosed. The compounds fall within a genus described by formula I

This application claims the benefit of U.S. Provisional Application No. 61/101,777, filed on Oct. 1, 2008 and U.S. Provisional Application No. 61/220,755, filed on Jun. 26, 2009, both of which are hereby incorporated by reference.

### FIELD OF THE INVENTION

The invention relates to modulators of chemoattractant receptor-homologous molecule expressed on T helper type 2 cells (CRTH2), particularly CRTH2 antagonists, that are useful for treating various disorders, including asthma and respiratory disorders.

### BACKGROUND OF THE INVENTION

CRTH2 is a Gαᵢ protein-coupled receptor involved in both mediating PGD2-induced chemoattraction and in activation of specific cell types involved in allergic inflammation. CRTH2 is expressed by Th2 cells, eosinophils and basophils, but not by Th1 cells, B cells or NK cells. PGD2 is produced by allergen-activated mast cells and has been implicated in various allergic diseases as a pro-inflammatory mediator, such as asthma, rhinitis and allergies. Thus, blocking binding of PGD2 to CRTH2 is a useful therapeutic strategy for treatment of such diseases.

CRTH2 agonists activate eosinophils, basophils and Th2 cells in vitro, resulting in induction of actin polymerization, calcium influx, CD11b expression and chemotaxis. Injection of a CRTH2 agonist in vivo can elicit transient recruitment of eosinophils from bone marrow into the blood. A genetic study of African American and Chinese cohorts found that polymorphisms in CRTH2 were tightly associated with asthma susceptibility. Thus, it has been suggested that modulators of CRTH2, particularly CRTH2 inhibitors, may be useful in the prevention and/or treatment of allergic asthma and other allergic disorders as recruitment and/or activation of eosinophils, basophils and Th2 cells is a prominent feature of the changes that occur in the asthmatic lung. Similar activation of these cell types, or subsets thereof, is believed to play an important role in the etiology of other diseases, including eosinophilic esophagitis and atopic dermatitis. This fact, combined with the fact that CRTH2 mediates PGD₂-induced chemotaxis, suggests that compounds that alter chemotaxis by inhibiting CRTH2 activity could be useful in controlling various diseases and disorders, including, without limitation, allergic asthma, chronic airway inflammation, atopic dermatitis, chronic obstructive pulmonary disease (COPD), and/or eosinophilic esophagitis.

Compounds that alter chemotaxis by inhibiting CRTH2 activity could also be useful in controlling allergic rhinitis, which is classified as either seasonal (SAR) or perennial (PAR) depending upon the type of trigger and duration of symptoms. SAR symptoms occur in the spring, summer and/or early fall and can be triggered by outdoor allergens such as airborne tree, grass and weed pollens while PAR is usually persistent and chronic with symptoms occurring year-round and is commonly associated with indoor allergens such as dust mites, animal dander and/or mold spores. Symptoms of allergic rhinitis may include runny nose, nasal itching, sneezing, watery eyes and nasal congestion.

CRTH2 agonists can induce desensitization of the cell system by promoting internalization and down regulation of the cell surface receptor. For example, certain CRTH2 agonists can induce desensitization of PGD₂-responsive cells to subsequent activation by a CRTH2 agonist. Therefore, CRTH2 modulators that are CRTH2 agonists may be therapeutically useful because they can cause the desensitization of PGD₂-responsive cells. Importantly, CRTH2 agonists may also cause cross-desensitization. Cross-desensitization, which can occur in many cell-signaling systems, refers to a phenomenon whereby an agonist for one receptor can reduce or eliminate sensitivity of a cell type to an unrelated agonist/receptor signaling system. For example, treatment with the CRTH2 agonist indomethacin reduces expression of CCR3, the receptor for the chemoattractant, eotaxin.

CRTH2 is also found on cell types outside the immune system, including spinal cord neurons and brain. PGD₂ activation of CRTH2, e.g., during inflammation, can lead to hyperalgesia, allodynia and neuropathic pain. Thus, inhibitors of CRTH2 may be used to treat hyperalgesia, allodynia and neuropathic pain.

Accordingly, there is a need to develop inhibitors of CRTH2, which could be used to prevent and/or treat disorders such as allergic rhinitis, asthma, chronic airway inflammation, atopic dermatitis, chronic obstructive pulmonary disease (COPD), eosinophilic esophagitis and/or neuropathic pain.

### SUMMARY OF THE INVENTION

It has now been found that compounds of this invention, and pharmaceutically acceptable compositions thereof, are effective as inhibitors of CRTH2. Thus, in one aspect the present invention relates to compounds of formula I

wherein
A is a 5, 6, 7, 8, 9 or 10-membered non-aromatic carbocycle; wherein A is optionally substituted with up to eight instances of R⁸;
L is chosen from
   —O—,
   —S(O)ₘ—,
   —NR¹⁴— and
   (C₁-C₃)alkylene, wherein, when said (C₁-C₃)alkylene is a C₂- or C₃-alkylene, one CH₂ is optionally replaced by —O—, —S(O)ₘ— or —NR¹⁴—, and wherein one or more substitutable carbon atoms of said (C₁-C₃)alkylene is optionally substituted with up to three instances of R¹¹;
X is chosen from a direct bond and (C₁-C₂)alkylene, wherein said (C₁-C₂)alkylene is optionally substituted with up to two instances of R¹²;
R¹ is chosen from (3-8-membered)carbocyclyl, (3-8-membered) heterocyclyl, —NR⁶(C₁-C₆)alkyl, —NR⁶(3-8-membered)carbocyclyl and —NR⁶(3-8-membered)heterocyclyl; wherein R¹ is optionally substituted with up to four instances of R⁹;
R² is chosen from hydrogen, halogen, (C₁-C₄)alkyl, (C₁-C₄)haloalkyl, and OH;
R³ is chosen from hydrogen, halogen, (C₁-C₄)alkyl and (C₁-C₄)haloalkyl;
R⁴ is chosen from hydrogen, halogen, (C₁-C₄)alkyl and (C₁-C₄)haloalkyl; or,
R³ and R⁴, taken together, form a (C₃-C₇)cycloalkyl ring;
R⁵ is chosen from C(O)OR⁷, C(O)N(R⁷)₂, C(O)NOR⁷ and C(O)NSR⁷;
R⁶ is chosen from H and (C₁-C₆)alkyl;
R⁷ is selected from hydrogen and (C₁-C₄)alkyl, wherein said (C₁-C₄)alkyl is optionally substituted with up to four instances of R¹⁶;
R⁸ in each occurrence is independently selected from halogen, (C₁-C₄)alkyl, (C₁-C₄)haloalkyl, (C₁-C₄)alkoxy, (C₁-C₄)haloalkoxy, CN, OH, oxo, and N(R¹⁴)₂;
R⁹ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁴)₂;
R¹⁰ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;
R¹¹ in each occurrence is independently selected from halogen, (C₁-C₄)alkyl, (C₁-C₄)haloalkyl, (C₃-C₆)cycloalkyl, CN, OH, (C₁-C₄)alkoxy and (C₁-C₄)haloalkoxy;
R¹² in each occurrence is independently selected from halogen, (C₁-C₄)alkyl and (C₁-C₄)haloalkyl;
R¹⁴ is selected from hydrogen and (C₁-C₄)alkyl, wherein said (C₁-C₄)alkyl is optionally substituted with up to four instances of R¹⁰;
R¹⁵ is selected from hydrogen and (C₁-C₄)alkyl;
R¹⁶ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;
m is zero, one or two; and
n is zero, one or two.

In another aspect, the invention relates to a composition comprising a pharmaceutically acceptable carrier and a compound as described above.

In another aspect, the invention relates to a method for treating a patient suffering from a disease involving the CRTH2 receptor. The method comprises administering to the patient a therapeutically effective amount of a compound as described above. Typical diseases that involve the CRTH2 receptor and that can be treated with the compounds described above include, without limitation, asthma, allergic rhinitis and chronic obstructive pulmonary disease (COPD).

### DETAILED DESCRIPTION OF THE INVENTION

As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, which are herein incorporated by reference in their entirety.

Unless otherwise specified, alkyl is intended to include linear chain, branched chain, or cyclic hydrocarbon structures and combinations thereof. A combination would be, for example, cyclopropylmethyl. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Hydrocarbon refers to any substituent comprised of hydrogen and carbon as the only elemental constituents. C₁ to C₂₀ hydrocarbon includes alkyl, cycloalkyl, polycycloalkyl, alkenyl, alkynyl, aryl and combinations thereof. Examples of C₁ to C₂₀ hydrocarbon include benzyl, phenethyl, cyclohexylmethyl, camphoryl and naphthylethyl. Unless otherwise specified, alkenyl is intended to include linear chain, branched chain or cyclic unsaturated hydrocarbon groups have at least carbon to carbon double bond, but no carbon to carbon triple bonds. Unless otherwise specified, alkynyl is intended to include linear chain, branched chain or cyclic unsaturated hydrocarbon groups have at least one carbon to carbon triple bond, wherein the alkynyl optionally can have one or more carbon to carbon double bonds.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Alkoxy or alkoxyl refers to groups of from 1 to 8 carbon atoms of a straight, branched, cyclic configuration and combinations thereof attached to the parent structure through an oxygen. Examples of alkoxy or alkoxyl include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons have been replaced by sulfur or nitrogen, respectively. Examples include ethylaminoethyl and methylthiopropyl.

Unless otherwise specified, acyl refers to formyl and to groups of 1, 2, 3, 4, 5, 6, 7 or 8 carbon atoms of a straight chain, branched chain, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself is called “oxo”.

Aryl means (i) a monocyclic 6-membered aromatic ring; (ii) a bicyclic 9- or 10-membered aromatic reing system; or (iii) a tricyclic 13- or 14-membered aromatic or ring system. Heteroaryl mean (i) a monocyclic 5- or 6-membered heteroaromatic ring containing 1-3 heteroatoms selected from O, N, or S or a phenyl group (or benzene); (ii) a bicyclic 9- or 10-membered aromatic or heteroaromatic ring system containing 1-4 heteroatoms selected from O, N, or S; or (iii) a tricyclic 13- or 14-membered aromatic or heteroaromatic ring system containing 1-5 heteroatoms selected from O, N, or S. The aromatic 6- to 14-membered carbocyclic rings include, e.g., benzene, naphthalene, indane, tetralin, and fluorene. 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl refers to a substituent in which an aryl residue is attached to the parent structure through alkyl. Examples of arylalkyl are benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Examples of heteroarylalkyl include, e.g., pyridinylmethyl, pyrimidinylethyl and the like.

Heterocycle means a cycloalkyl or aryl carbocycle residue in which from one to three carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

As used herein, the term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, heterocyclyl, etc., refer to alkyl, aryl, cycloalkyl, or heterocyclyl wherein one or more H atoms in each alkyl, aryl, cycloalkyl or heterocyclyl residue are replaced with halogen, haloalkyl, alkyl, acyl, alkoxyalkyl, hydroxyloweralkyl, phenyl, heteroaryl, benzenesulfonyl, hydroxy, loweralkoxy, haloalkoxy, carboxy, alkoxycarbonyl [—C(═O)O-alkyl], alkoxycarbonylamino [HNC(═O)O-alkyl], carboxamido [—C(═O)NH₂], alkylaminocarbonyl [—C(═O)NH-alkyl], cyano, acetoxy, nitro, amino, alkylamino, dialkylamino, mercapto, alkylthio, sulfoxide, sulfone, sulfonylamino, acylamino, amidino, aryl, benzyl, heterocyclyl, phenoxy, benzyloxy, heteroaryloxy, hydroxyimino, alkoxyimino, oxaalkyl, aminosulfonyl, trityl, amidino, guanidino, ureido, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g. on phenyl). In one embodiment, 1, 2 or 3 hydrogen atoms of any one of the alkyl, aryl, cycloalkyl and heterocyclyl residues are replaced with the above specified substituents.

The terms “haloalkyl” and “haloalkoxy” mean alkyl or alkoxy, respectively, substituted with one or more halogen atoms. The terms “alkylcarbonyl” and “alkoxycarbonyl” mean —C(═O)alkyl or —C(O)alkoxy, respectively.

The term “halogen” means fluorine, chlorine, bromine or iodine. In one embodiment, halogen may be fluorine or chlorine.

Substituents Rⁿ are generally defined when introduced and retain that definition throughout the specification and in all independent claims.

When used in a structural or chemical formula, “Et” refers to an ethyl group (—CH₂CH₃), “Me” refers to a methyl group (—CH₃), and “Ph” refers to a phenyl group (—C₆H₆).

As used herein, and as would be understood by the person of skill in the art, the recitation of “a compound”—unless expressly further limited—is intended to include salts, solvates and inclusion complexes of that compound. Thus, for example, the recitation “a compound of formula I” as depicted above, in which R⁵ is COOH, would include salts in which R⁵ is COO⁻ M⁺, wherein M is any counterion. In a particular embodiment, the term “compound of formula I” refers to the compound of formula I or a pharmaceutically acceptable salt thereof. Unless otherwise stated or depicted, structures depicted herein are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen atoms by deuterium or tritium, the replacement of a carbon atom by a ¹³C- or ¹⁴C-enriched carbon atom or the replacement of a sulfur atom by a ³⁵S-enriched sulfur atom are within the scope of this invention. Such compounds are useful, for example, as analytical tools or probes in biological assays.

In one aspect, the invention relates to a compound of formula I as defined above.

Alternatively, A may be a fused cycloheptyl ring optionally substituted with up to eight instances of R⁸, independently selected. The compounds of this embodiment may be represented by the formula:

in which p is zero or an integer from 1 to 8.

In another embodiment, A may be a fused cyclopentyl ring optionally substituted with up to six instances of R⁸, independently selected. The compounds of this embodiment may be represented by the formula:

in which q is zero or an integer from 1 to 6.

In another embodiment, A may be a fused cyclohexyl ring optionally substituted with up to eight instances of R⁸, independently selected. The compounds of this embodiment may be represented by the formula:

In certain embodiments, two R⁸ may each be a methyl residue attached to the same ring carbon, such as in compounds of formula

in which t is zero or an integer from 1 to 4. In a particular embodiment, t is zero. In another embodiment, t is 1 and R⁸ is oxo.

In certain embodiments X may be chosen from a direct bond, —CH₂— and —CH₂CH₂—. Alternatively, X may be a direct bond.

In certain embodiments R² may be chosen from hydrogen, fluoro, methyl, ethyl and trifluoromethyl. Alternatively, R² may be methyl.

In certain embodiments, R³ and R⁴ may be taken together to form a cyclopropyl ring. In other embodiments R³ and R⁴ may each be independently selected from hydrogen and methyl and the methyl may be substituted with 1-3 instances of halogen, particularly fluoro. In other embodiments, R³ and R⁴ may each be hydrogen.

Preferably, L may be chosen from —CH₂—, —O—, —S—, —SO— and —SO₂—. More preferably, L is —CH₂—.

Preferably, X is a direct bond and R⁵ is selected from the group consisting of C(O)OR⁷, C(O)N(R⁷)₂, C(O)NHOR⁷ or C(O)NHSR⁷, and R⁷ may be H or (C₁₋₄)alkyl. More preferably, R⁵ may be either C(O)OH or C(O)O(C₁₋₄)alkyl. When R⁵ is C(O)OH, the compounds of the invention may be presented in the form of salts. Salts containing pharmaceutically acceptable cations are preferred for compositions and formulations that will be administered to humans.

In certain embodiments, R¹ is preferably —NR⁶(C₁-C₆)alkyl and R⁶ is preferably hydrogen or methyl. In other embodiments R¹ may be a non-aromatic 3-8 membered heterocycle, phenyl, or a non-aromatic 3-8 membered carbocycle, and the heterocycle, phenyl or carbocycle may be substituted with up to four instances of R⁹. In some embodiments, R¹ may be phenyl or a non-aromatic 3-8 membered carbocycle; in others, R¹ is a non-aromatic 3-8 membered heterocycle; in others, R¹ is a non-aromatic 5-7 membered heterocycle, optionally substituted with one to three instances of R⁹. In particular embodiments, R¹ is an N-attached pyrrolidine, piperidine, piperazine, azepine or morpholine, optionally substituted with one to three instances of R⁹, wherein each R⁹ is independently selected from (C₁-C₄)alkyl and (C₁-C₄)haloalkyl. In certain embodiments, R¹ is an N-attached piperazine of formula

in which R¹³ is chosen from hydrogen, (C₁-C₄)alkyl, (C₁-C₄)alkylcarbonyl and (C₁-C₄)alkoxycarbonyl and u is zero, one or two. In other embodiments, R¹ is an N-attached morpholine of formula

and u is zero, one or two. The compound may have one of the following formulae:

wherein p may be zero or an integer from 1 to 3. The compound may have the formula:

and p may be zero or one.

In many embodiments shown above, —S(O)ₙR¹ is attached para or ortho to L on the phenyl ring. As before, R⁵ may be C(O)OH or C(O)O(C₁₋₄)alkyl, and when R⁵ is C(O)OH the compounds of the invention may be presented in the form of salts.

Compounds of the invention may comprise compounds of formula I in which
A is chosen from a cyclopentyl ring, a cycloheptyl ring and a dimethylcyclohexyl ring;
R² may be methyl;
R³ and R⁴ may be hydrogen;
R⁵ may be COOH;
X may be a direct bond;
L may be —CH₂—;
n may be 2; and
R¹ may be chosen from
   (a) pyrrolidinyl, piperidinyl, azepinyl or morpholinyl;
   (b) piperazine-1-yl substituted at the 4 position with (C₁-C₄)alkylcarbonyl or (C₁-C₄)alkoxycarbonyl;
   (c) —NR⁶(C₁-C₆)alkyl wherein R⁶ is hydrogen or methyl; and
   (d) phenyl and substituted phenyl.

In another embodiment, the invention comprises compounds of formula II

in which
A may be chosen from a cyclopentyl ring, a cycloheptyl ring, a cyclohexyl ring, and a dimethylcyclohexyl ring;
R⁷ may be hydrogen or (C₁₋₄) alkyl;
L may be —CH₂— or —S(O)ₘ—;
m may be 0 or 2;
R⁸ may be oxo;
t may be 0 or 1; and
R¹ may be chosen from
   (a) pyrrolidinyl, piperidinyl, azepinyl or morpholinyl;
   (b) piperazine-1-yl substituted at the 4 position with (C₁-C₄)alkylcarbonyl or (C₁-C₄)alkoxycarbonyl;
   (c) —NR⁶(C₁-C₆)alkyl wherein R⁶ is hydrogen or methyl; or
   (d) phenyl and substituted phenyl.

In a further embodiment of compounds of formula II, A may be a cyclohexyl ring or a dimethylcyclohexyl ring.

In a further embodiment, the invention may comprise compounds of formulae II′ and II″:

In the compounds of any of formulae II, II′ or II″, L may be —CH₂—.

In the compounds of any of formulae II, II′ or II″, t may be one and R⁸ may be oxo. In yet a further embodiment, the invention comprises compounds of formula III:

In the compounds of any of formulae II, II′, II″ or III, R¹ may be chosen from pyrrolidinyl, piperidinyl, azepinyl, morpholinyl or phenyl. Alternatively, in the compounds of any of formulae II, II′, II″ or III, R¹ may be chosen from pyrrolidine or morpholine.

In the compounds of any of formulae II, II′, II″ or III, —S(O)₂R¹ may be attached para or ortho to L on the phenyl ring. As before, when R⁷ is hydrogen, the compounds of the invention may be presented in the form of salts.

In one embodiment, of compounds II, the fused cyclohexyl ring may be substituted with up to eight instances of R⁸ independently selected from the group consisting of halogen and (C₁-C₄)alkyl. In this embodiment the compound preferably has one of the following three formulae:

wherein t is 0 or an integer of from 1 to 6. Preferably R⁸ is selected from the group consisting of fluoro and methyl. Preferably, t is an integer from 1 to 4. In this embodiment, preferably the compound has the following structure:

wherein p is 0 or an integer from 1 to 4. Most preferably, p is 0 or R₈ is an Oxo.

Further examples of embodiments of the invention are shown in Table 1 below.

```
TABLE 1
  1-1
  1-2
  1-3
  1-4
  1-5
  1-6
  1-7
  1-8
  1-9
  1-10
  1-11
  1-12
  1-13
  1-14
  1-15
  1-16
  1-17
  1-18
  1-19
  1-20
  1-21
  1-22
  1-23
  1-24
  1-25
  1-26
  1-27
  1-28
  1-29
  1-30
  1-31
  1-32
  1-33
  1-34
  1-35
  1-36
  1-37
  1-38
  1-39
  1-40
  1-41
  1-42
  1-43
  1-44
  1-45
  1-46
  1-47
  1-48
  1-49
  1-50
  1-51
  1-52
  1-53
  1-54
  1-55
  1-56
  1-57
  1-58
  1-59
  1-60
  1-61
  1-62
  1-63
  1-64
  1-65
  1-66
  1-67
  1-68
  1-69
  1-70
  1-71
  1-72
  1-73
  1-74
  1-75
  1-76
  1-77
  1-78
  1-79
  1-80
  1-81
  1-83
  1-82
  1-84
  1-85
  1-86
  1-87
  1-92
  1-93
  1-95
  1-96
  1-97
  1-99
  1-100
  1-101
  1-102
  1-103
  1-104
  1-105
```

The compounds of the invention preferably are CRTH2 antagonists that can be used, for example, to prevent and/or treat conditions or disorders in which it is considered desirable to reduce or eliminate CRTH2 activity. CRTH2 antagonists may be used to aid in preventing and/or treating a disease or disorder mediated, regulated or influenced by, for example, Th2 cells, eosinophils, basophils, platelets, Langerhans cells, dendritic cells or mast cells. They also may be used to aid in the prevention or treatment of a disease or disorder mediated, regulated or influenced by PGD₂ and metabolites thereof, such as 13,14-dihydro-15-keto-PGD₂ and 15-deoxy-Al 2,1′-PGD₂.

CRTH2 antagonists may be useful in the prevention and/or treatment of disease and disorders characterized by undesirable activation of Th2 cells, eosinophils, and basophils e.g., asthma, atopic dermatitis, allergic rhinitis, allergies (e.g., food allergies, dust allergies, pollen allergies, mold allergies), and Grave's Disease. CRTH2 antagonists or agonists may be used to aid in preventing and/or treating the following types of diseases, conditions and disorders:

(1) respiratory tract/obstructive airways diseases and disorders including: acute-, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), antitussive activity, asthma (such as bronchial, allergic, intrinsic, extrinsic and dust asthma particularly chronic or inveterate asthma (e.g. late asthma and airways hyper-responsiveness)), bronchitis (including chronic and eosinophilic bronchitis), chronic inflammatory diseases of the lung which result in interstitial fibrosis, such as interstitial lung diseases (ILD) (e.g., idiopathic pulmonary fibrosis, or ILD associated with rheumatoid arthritis, scleroderma lung disease, or other autoimmune conditions), chronic obstructive pulmonary disease (COPD) (such as irreversible COPD), chronic sinusitis, conjunctivitis (e.g. allergic conjunctivitis), cystic fibrosis, fanner's lung and related diseases, fibroid lung, hypersensitivity lung diseases, hypersensitivity pneumonitis, idiopathic interstitial pneumonia, nasal congestion, nasal polyposis, otitis media, and chronic cough associated with inflammation or iatrogenic induced;

(2) systemic anaphylaxis or hypersensitivity responses, drug allergies (e.g., to penicillin, cephalosporins), insect sting allergies, and food related allergies which may have effects remote from the gut (such as migraine, rhinitis and eczema);

(3) bone and joint related diseases and disorders including: arthritis including rheumatic, infectious, autoimmune, seronegative, spondyloarthropathies (such as ankylosing spondylitis, psoriatic arthritis and Reiter's disease), osteoarthritis, and systemic sclerosis;

(4) skin and eye related diseases and disorders including: psoriasis, atopical dermatitis, contact dermatitis, other eczmatous, dermitides, seborrheic dermatitis, cutaneous eosinophilias, chronic skin ulcers, cutaneous lupus erythematosus, contact hypersensitivity/allergic contact dermatits (including sensitivity to poison ivy, sumac, or oak), and eosinophilic folliculitis (Ofuji's disease);

(5) gastrointestinal tract related diseases and disorders including: Coeliac disease, cholecystitis, Crohn's disease, enteritis (including eosinophilic gastroenteritis), eosinophilic esophagitis, enteropathy associated with seronegative arthropathies, gastritis, inflammatory bowel disease and irritable bowel disease;

(6) transplant rejection related conditions including: acute and chronic allograft rejection following solid organ transplant, for example, transplantation of kidney, heart, liver, lung, and cornea, chronic graft versus host disease, skin graft rejection, and bone marrow transplant rejection;

(7) inflammation;

(8) hyperalgesia, allodynia and neuropathic pain; and

(8) other diseases and disorders including: lupus erythematosus; systemic lupus, erythematosus; Hashimoto's thyroiditis, Grave's disease, type I diabetes, eosinophilia fasciitis, hyper IgE syndrome, idiopathic thrombocytopenia pupura; post-operative adhesions, ischemic/reperfusion injury in the heart, brain, peripheral limbs hepatitis (alcoholic, steatohepatitis and chronic viral), mastocytosis (cutaneous and systemic), mastitis (mammary gland), vaginitis, vasculitis (e.g., necrotizing, cutaneous, and hypersensitivity vasculitis), myositis (including polyinyositis, derinatomyositis), basophil related diseases including basophilic leukemia and basophilic leukocytosis, and eosinophil related diseases such as Churg-Strauss syndrome.

The compounds of the invention may be used alone or in combination with other compounds useful in the prevention and/or treatment of diseases and disorders for which compounds of the invention are useful, including any of the diseases or disorders described above. Examples of other therapeutic agents that may be combined with a compound of the invention, either administered separately or in the same pharmaceutical compositions, include, but are not limited to:

(1) inactivating antibodies (e.g., monoclonal or polyclonal) to interleukins (e.g., IL-4 and IL-5 (for example see Leckie et al. 2000 Lancet 356:2144));

(2) soluble chemokine receptors (e.g. recombinant soluble IL-4 receptor (Steinke and Borish 2001 Respiratory Research 2:66));

(3) chemokine receptor modulators including but not limited to antagonists of CCR1 (e.g., CP-481,715 (Gladue et al. J Biol Chem 278:40473)), CCR3 (e.g., UCB35625 (Sabroe et al. J Biol Chem 2000 275:25985), CCR5 and those described in: WO0039125A1, WO02070523A1, WO03035627A1, WO03084954A1, WO04011443A1, WO04014875A1, WO04018425A1, WO04018435A1, WO04026835A1, WO04026880A1, WO04039376A1, WO04039377A1, WO04039787A1, WO04056773A1, WO04056808A1, and WO04056809A1;

(4) histamine HI receptor antagonists/antihistamines (i.e. any compound that is capable of blocking, inhibiting, reducing or otherwise interrupting the interaction between histamine and its receptor) including but not limited to: —4 asternizole, acrivastine, antazoline, asternizole, azatadine, azelastine, bromopheniramine, carbinoxamine, carebastine, cetirizine, chlorpheniramine, clemastine, cyclizine, cyproheptadine, descarboethoxyloratadine, dexchlorpheniramine, dimethindene, diphenhydramine, diphenylpyraline, doxylarnine, ebastine, efletirizine, epinastine, fexofenadine, hydroxyzine, hydroxyzine, ketotifen, levocabastine, levocetirizine, levocetirizine, loratadine, meclizine, mequitazine, methdilazine, mianserin, mizolastine, noberastine, norasternizole, noraztemizole, pheniramine, picumast, promethazine, pyrilamine, temelastine, terfenadine, trimeprazine, tripelenamine, and triprolidin; leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (Singulair®), pranlukast, iralukast, pobilukast, SKB-106,203 and compounds described as having LTD4 antagonizing activity described in U.S. Pat. No. 5,565,473;

(5) PGD2 receptor antagonists including, but not limited to, compounds described as having PGD2 antagonizing activity in United States Published Applications US20020022218, US20010051624, and US20030055077, PCT Published Applications WO9700853, WO9825919, WO03066046, WO03066047, WO03101961, WO03101981, WO04007451, WO0178697, WO04032848, WO03097042, WO03097598, WO03022814, WO03022813, and WO04058164, European Patent Applications EP945450 and EP944614, and those listed in: Torisu et al. 2004 Bioorg Med Chem Lett 14:4557, Torisu et al. 2004 Bioorg Med Chem Lett 2004 14:4891, and Torisu et al. 2004 Bioorg & Med Chem 2004 12:4685;

(6) VLA-4 antagonists;

(7) corticosteroids, such as beclomethasone, methylprednisolone, betamethasone, prednisone, prenisolone, triamcinolone, dexamethasone, fluticasone, flunisolide and hydrocortisone, and corticosteroid analogs such as budesonide;

(8) immunosuppressants such as cyclosporine (cyclosporine A, Sandimmune® Neoral®), tacrolimus (FK-506, Prograf®), rapamycin (sirolimus, Rapamune®) and other FK-506 type immunosuppressants, and mycophenolate, e.g., mycophenolate mofetil (CellCept®);

(9) non-steroidal anti-asthmatics such as 132-agonists (e.g., terbutaline, metaproterenol, fenoterol, isoetharine, albuterol, salmeterol, bitolterol and pirbuterol) and J32-agonist-corticosteroid combinations (e.g., salmeterol-fluticasone (Advair®), formoterol-budesonid (Symbicort®)), theophylline, cromolyn, cromolyn sodium, nedocromil, atropine, ipratropium, ipratropium bromide, leukotriene biosynthesis inhibitors (zileuton, BAY1005);

(10) non-steroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone);

(11) cyclooxygenase-2 (COX-2) inhibitors such as celecoxib (Celebrex®), rofecoxib (Vioxx®), valdecoxib, etoricoxib, parecoxib and lumiracoxib;

(12) inhibitors of phosphodiesterase type IV (PDE-IV);

(13) opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine;

(14) antithrombotic agents, such as thrombolytic agents (e.g., streptokinase, alteplase, anistreplase and reteplase), heparin, hirudin and warfarin derivatives, β-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat);

(15) anti-diabetic agents such as insulin and insulin mimetics, sulfonylureas (e.g., glyburide, meglinatide), biguanides, e.g., metformin (Glucophage®), α-glucosidase inhibitors (acarbose), thiazolidinone compounds, e.g., rosiglitazone (Avandia®), troglitazone (Rezulin®), ciglitazone, pioglitazone (Actos®) and englitazone;

(16) preparations of interferon beta (interferon β-I α, interferon β-I β);

(17) gold compounds such as auranofin and aurothioglucose;

(18) TNF inhibitors, e.g., etanercept (Enbrel®), antibody therapies such as orthoclone (OKT3), daclizumab (Zenapax®), basiliximab (Simulec®)), infliximab (Remicade®) and D2E6 TNF antibody;

(19) lubricants or emollients such as petrolatum and lanolin, keratolytic agents, vitamin D₃ derivatives (e.g., calcipotriene and calcipotriol (Dovonex®)), PUVA, anthralin (Drithrocreme®), etretinate (Tegison®) and isotretinoin;

(20) multiple sclerosis therapeutic agents such as interferon β-I β (Betaseron®), interferon β-I α (Avonex®), azathioprine (Imurek®, Imuran®), glatiramer acetate (Capoxone®), a glucocorticoid (e.g., prednisolone) and cyclophosphamide; and

(21) other compounds such as 5-aminosalicylic acid and prodrugs thereof DNA-alkylating agents (e.g., cyclophosphamide, ifosfamide), antimetabolites (e.g., azathioprine, 6-mercaptopurine, methotrexate, a folate antagonist, and 5-fluorouracil, a pyrimidine antagonist), microtubule disruptors (e.g., vincristine, vinblastine, paclitaxel, colchicine, nocodazole and vinorelbine), DNA intercalators (e.g., doxorubicin, daunomycin and cisplatin), DNA synthesis inhibitors such as hydroxyurea, DNA cross-linking agents, e.g., mitomycin C, hormone therapy (e.g., tamoxifen, and flutamide), and cytostatic agents, e.g., imatinib (STI571, Gleevec®) and rituximab (Rituxan®).

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. The reader's attention is directed to the Physician's Desk Reference, a standard text in the field, in which the term “prevent” occurs hundreds of times. No person of skill in the medical art construes the term in an absolute sense.

The compounds of formula (I) are preferably administered as a pharmaceutical composition. According to a further aspect, the present invention provides a pharmaceutical composition comprising a compound of formula (I) or a pharmaceutically acceptable salt or solvate thereof, together with one or more pharmaceutically acceptable carriers thereof and optionally one or more other therapeutic ingredients. The carrier(s) is “acceptable” in the sense of being compatible with the other ingredients of the formulation and not deleterious to the recipient thereof. For example, compounds of formula (I), optionally with one or more other therapeutic ingredients, can be combined with one or more carriers or mediums that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Examples of salts derived from inorganic bases include aluminum, ammonium, calcium, copper, ferric, ferrous, lithium, magnesium, manganic salts, manganous, potassium, sodium, zinc, and the like. In some embodiments, the salt may be an ammonium, calcium, magnesium, potassium, or sodium salt. Examples of salts derived from inorganic bases include aluminum, ammonium, calcium, copper, ferric, ferrous, lithium, magnesium, manganic salts, manganous, potassium, sodium, zinc, and the like. In some embodiments, the salt may be an ammonium, calcium, magnesium, potassium, or sodium salt. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, e.g. R⁵, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include tris, arecoline, arginine, barium, betaine, bismuth, chloroprocaine, choline, clemizole, deanol, imidazole, and morpholineethanol. In one embodiment are tris salts.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula (I) or a pharmaceutically acceptable salt or solvate thereof (“active ingredient”) with the carrier which constitutes one or more accessory ingredients. In general, the formulations may be prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

Preferred unit dosage formulations are those containing an effective dose, as hereinbelow recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose from about 0.001 to about 2500 mg/kg per day. The dose range for adult humans is preferably from about 0.005 mg to about 10 g/day. Tablets or other forms of presentation provided in discrete units may conveniently contain an amount of compound of the invention which is effective at such dosage or as a multiple of the same, for instance, units containing from about 5 mg to about 500 mg, usually from about 10 mg to about 200 mg.

The compounds of formula (I) may be administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

The compounds of formula (I) may be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents may also be administered by captisol delivery technology, rectal suppository or parenterally.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein. The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: alginic acid, cellulose and its derivatives (e.g. ethyl cellulose, cellulose acetate, carboxymethyl cellulose, carboxymethyl cellulose calcium, sodium carboxymethyl cellulose), citric acid monohydrate, corn starch, gelatin, guar gum, hydroxymethyl cellulose, hydroxypropyl cellulose, hydroxypropyl methyl cellulose, methyl cellulose, microcrystalline cellulose (e.g. AVICEL™ such as AVICEL-PH-101™, -103™, and 105™ sold by FMC Corporation, Marcus Hook, Pa. USA), natural and synthetic gums such as acacia, other alginates, other starches, polyethylene oxide, polyvinyl alcohol, polyvinyl pyrrolidone, potato starch, powdered tragacanth, pre-gelatinized starch (e.g. STARCH 1500® and STARCH 1500 LM®, sold by Colorcon), sodium alginate, or mixtures thereof;

FILLERS: aluminum magnesium hydroxide, aluminum oxide, calcium carbonate (e.g. granules or powder), calcium dihydroxide, calcium sulfate (e.g. granules or powder), dextrates, dextrose, dibasic calcium phosphate, dibasic calcium phosphate anhydrous, fructose (granules or powder), honey, hydrous lactose, iron oxides (e.g. yellow, black, red, e.g. ferric oxide), kaolin, lactose, lactose and aspartame, lactose and cellulose, lactose and microcrystalline cellulose, lactose anhydrate, lactose monohydrate, magnesium aluminate, magnesium carbonate, magnesium hydroxide, maltodextrin, maltose, mannitol, microcrystalline cellulose, microcrystalline cellulose & guar gum, molasses, powdered cellulose, pre-gelatinized starch, silicic acid, silicic anhyride, silicified microcrystalline cellulose, sodium choloride, sorbitol, soybean lecithin, starch, sucrose, talc, triacetin, tribasic calcium phosphate, xanthar gum, or mixtures thereof;

DISINTEGRANTS: agar-agar, alginic acid, calcium carbonate, clays, croscarmellose sodium, crospovidone, gums (like gellan), lactose monohydrate, low-substituted hydroxypropyl cellulose, microcrystalline cellulose, other algins, other celluloses, other starches, polacrilin potassium, potato or tapioca starch, povidone, pre-gelatinized starch, simethicone emulsion, sodium starch glycolate, or mixtures thereof;

SURFACTANTS: Tween 80 or polyoxyethylene-polyoxypropylene copolymer, polyoxyethylene sorbitan, or mixtures thereof;

LUBRICANTS: a coagulated aerosol of synthetic silica (Degussa Co. Plano Tex. USA), a pyrogenic silicon dioxide (CAB-O-SIL, Cabot Co., Boston, Mass. USA), agar, calcium stearate, ethyl laurate, ethyl oleate, glycerin, hydrogenated vegetable oil (e.g., peanut oil, cottonseed oil, sunflower oil, sesame oil, olive oil, corn oil and soybean oil), light mineral oil, magnesium stearate, mannitol, mineral oil, other glycols, palmitic acid, polyethylene glycol, sodium lauryl sulfate, sodium stearyl fumarate, sorbitol, stearic acid, syloid silica gel (AEROSIL 200, W.R. Grace Co., Baltimore, Md. USA), talc, vegetable based fatty acids lubricant, zinc stearate, or mixtures thereof;

ANTI-CAKING AGENTS: calcium silicate, magnesium silicate, silicon dioxide, colloidal silicon dioxide, talc, or mixtures thereof,

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, polysorbate, sorbic acid, thimersol, thymo, or mixtures thereof;

COATING AGENTS: candellilla wax, carnuba wax, cellulose acetate phthalate, ethylcellulose, gelatin, gellan gum, hydroxypropyl cellulose, hydroxypropyl methyl cellulose phthalate, hydroxypropyl methylcellulose (hypromellose), maltodextrin, methacrylates, methylcellulose, microcrystalline cellulose and carrageenan, microcrystalline wax, pharmaceutical glaze, polyethylene glycol (e.g. polyethylene glycol 8000, polyethylene glycol 3000), polyvinyl acetate phthalate, shellac, sodium carboxymethyl cellulose, sucrose, titanium dioxide, or mixtures thereof; COLORANTS: FD&C blue no. 1, D&C yellow #10 aluminum lake, FD&C yellow #6/sunset yellow FCF aluminum lake, FD&C carmine aluminum lake and FD&C blue #1, or mixtures thereof; and

ANTIOXIDANTS: butylated hydroxyanisole, sodium ascorbate, sodium metabisulfate, malic acid, citric acid, ascorbic acid, butylated hydroxytoluene, vitamin C, propyl gallate, or mixtures thereof.

The formulation can also include other excipients and categories thereof including but not limited to L-histidine, Pluronic®, Poloxamers (such as Lutrol® and Poloxamer 188), ascorbic acid, glutathione, permeability enhancers (e.g. lipids, sodium cholate, acylcarnitine, salicylates, mixed bile salts, fatty acid micelles, chelators, fatty acid, surfactants, medium chain glycerides), protease inhibitors (e.g. soybean trypsin inhibitor, organic acids), pH lowering agents and absorption enhancers effective to promote bioavailability (including but not limited to those described in U.S. Pat. No. 6,086,918 and U.S. Pat. No. 5,912,014), creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD& C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, mannitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

Solid oral dosage forms may optionally be treated with coating systems (e.g. Opadry® fx film coating system, for example Opadry® blue (OY-LS-20921), Opadry® white (YS-2-7063), Opadry® white (YS-1-7040), and black ink (S-1-8106).

The dose range for adult humans is generally from 0.005 mg to 10 g/day orally. Tablets or other forms of presentation provided in discrete units may conveniently contain an amount of compound described herein which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g. an oral dosage unit) can include from, for example, 1 to 30 μg, 1 to 40 μg, 1 to 50 μg, 1 to 100 μg, 1 to 200 μg, 1 to 300 μg, 1 to 400 μg, 1 to 500 μg, 1 to 600 μg, 1 to 700 μg, 1 to 800 μg, 1 to 900 μg, 1 to 1000 μg, 10 to 30 μg, 10 to 40 μg, 10 to 50 μg, 10 to 100 μg, 10 to 200 μg, 10 to 300 μg, 10 to 400 μg, 10 to 500 μg, 10 to 600 μg, 10 to 700 μg, 10 to 800 μg, 10 to 900 μg, 10 to 1000 μg, 100 to 200 μg, 100 to 300 μg, 100 to 400 μg, 100 to 500 μg, 100 to 600 μg, 100 to 700 μg, 100 to 800 μg, 100 to 900 μg, 100 to 1000 μg, 100 to 1250 μg, 100 to 1500 μg, 100 to 1750 μg, 100 to 2000 μg, 100 to 2250 μg, 100 to 2500 μg, 100 to 2750 μg, 100 to 3000 μg, 200 to 300 μg, 200 to 400 μg, 200 to 500 μg, 200 to 600 μg, 200 to 700 μg, 200 to 800 μg, 200 to 900 μg, 200 to 1000 μg, 200 to 1250 μg, 200 to 1500 μg, 200 to 1750 μg, 200 to 2000 μg, 200 to 2250 μg, 200 to 2500 μg, 200 to 2750 μg, 200 to 3000 μg, 300 to 400 μg, 300 to 500 μg, 300 to 600 μg, 300 to 700 μg, 300 to 800 μg, 300 to 900 μg, 300 to 1000 μg, 300 to 1250 μg, 300 to 1500 μg, 300 to 1750 μg, 300 to 2000 μg, 300 to 2250 μg, 300 to 2500 μg, 300 to 2750 μg, 300 to 3000 μg, 400 to 500 μg, 400 to 600 μg, 400 to 700 μg, 400 to 800 μg, 400 to 900 μg, 400 to 1000 μg, 400 to 1250 μg, 400 to 1500 μg, 400 to 1750 μg, 400 to 2000 μg, 400 to 2250 μg, 400 to 2500 μg, 400 to 2750 μg, 400 to 3000 μg, 500 to 600 μg, 500 to 700 μg, 500 to 800 μg, 500 to 900 μg, 500 to 1000 μg, 500 to 1250 μg, 500 to 1500 μg, 500 to 1750 μg, 500 to 2000 μg, 500 to 2250 μg, 500 to 2500 μg, 500 to 2750 μg, 500 to 3000 μg, 600 to 700 μg, 600 to 800 μg, 600 to 900 μg, 600 to 1000 μg, 600 to 1250 μg, 600 to 1500 μg, 600 to 1750 μg, 600 to 2000 μg, 600 to 2250 μg, 600 to 2500 μg, 600 to 2750 μg, 600 to 3000 μg, 700 to 800 μg, 700 to 900 μg, 700 to 1000 μg, 700 to 1250 μg, 700 to 1500 μg, 700 to 1750 μg, 700 to 2000 μg, 700 to 2250 μg, 700 to 2500 μg, 700 to 2750 μg, 700 to 3000 μg, 800 to 900 μg, 800 to 1000 μg, 800 to 1250 μg, 800 to 1500 μg, 800 to 1750 μg, 800 to 2000 μg, 800 to 2250 μg, 800 to 2500 μg, 800 to 2750 μg, 800 to 3000 μg, 900 to 1000 μg, 900 to 1250 μg, 900 to 1500 μg, 900 to 1750 μg, 900 to 2000 μg, 900 to 2250 μg, 900 to 2500 μg, 900 to 2750 μg, 900 to 3000 μg, 1000 to 1250 μg, 1000 to 1500 μg, 1000 to 1750 μg, 1000 to 2000 μg, 1000 to 2250 μg, 1000 to 2500 μg, 1000 to 2750 μg, 1000 to 3000 μg, 2 to 500 μg, 50 to 500 μg, 3 to 100 μg, 5 to 20 μg, 5 to 100 μg, 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3250 μg, 3500 μg, 3750 μg, 4000 μg, 4250 μg, 4500 μg, 4750 μg, 5000 μg, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein. In certain embodiments the dosage unit and daily dose are equivalent. In various embodiments, the dosage unit is administered with food at anytime of the day, without food at anytime of the day, with food after an overnight fast (e.g. with breakfast), at bedtime after a low fat snack. In various embodiments, the dosage unit is administered once a day, twice a day, three times a day, four times a day.

Combining two or more active ingredients in single dosage form results in the possibility of chemical interactions between the active drug substances. For example, acidic and basic active ingredients can react with each other and acidic active ingredients can facilitate the degradation of acid labile substances. Thus, in certain dosage forms, acidic and basic substances can be physically separated as two distinct or isolated layers in a compressed tablet, or in the core and shell of a press-coated tablet. Additional agents that are compatible with acidic as well as basic substances, have the flexibility of being placed in either layer. In certain multiple layer compositions at least one active ingredient can be enteric-coated. In certain embodiments thereof at least one active ingredient can be presented in a controlled release form. In certain embodiments where a combination of three or more active substances are used, they can be presented as physically isolated segments of a compressed multilayer tablet, which can be optionally film coated.

The therapeutic combinations described herein can be formulated as a tablet or capsule comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

The therapeutic combinations described herein can be formulated as a capsule comprising microtablets or minitablets of all active ingredients. Microtablets of the individual agents can be prepared using well known pharmaceutical procedures of tablet making like direct compression, dry granulation or wet granulation. Individual microtablets can be filled into hard gelatin capsules. A final dosage form may comprise one or more microtablets of each individual component. The microtablets may be film coated or enteric coated.

The therapeutic combinations described herein can be formulated as a capsule comprising one or more microtablets and powder, or one or more microtablets and granules or beads. In order to avoid interactions between drugs, some active ingredients of a said combination can be formulated as microtablets and the others filled into capsules as a powder, granules, or beads. The microtablets may be film coated or enteric coated. At least one active ingredient can be presented in controlled release form.

The therapeutic combinations described herein can be formulated wherein the active ingredients are distributed in the inner and outer phase of tablets. In an attempt to divide chemically incompatible components of proposed combination, few interacting components are converted in granules or beads using well known pharmaceutical procedures in prior art. The prepared granules or beads (inner phase) are then mixed with outer phase comprising the remaining active ingredients and at least one pharmaceutically acceptable excipient. The mixture thus comprising inner and outer phase is compressed into tablets or molded into tablets. The granules or beads can be controlled release or immediate release beads or granules, and can further be coated using an enteric polymer in an aqueous or non-aqueous system, using methods and materials that are known in the art.

The therapeutic combinations described herein can be formulated as single dosage unit comprising suitable buffering agent. All powdered ingredients of said combination are mixed and a suitable quantity of one or more buffering agents is added to the blend to minimize possible interactions.

The agents described herein, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

### Formulation

The agents either in their free form or as a salt can be combined with a polymer such as polylactic-glycolic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(M-caprolactone) and poly(alkylene oxide) (U.S. 20030068384) to create a sustained release formulation. Such formulations can be used in implants that release a compound or another agent over a period of a few days, a few weeks or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and. US20020019446. In such sustained release formulations microparticles of compound are combined with microparticles of polymer. U.S. Pat. No. 6,011,011 and WO 94/06452 describe a sustained release formulation providing either polyethylene glycols (where PEG 300 and PEG 400 are most preferred) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1,U.S. Pat. No. 6,667,060, WO 01/49249, WO 01/49311, WO 01/49249, WO 01/49311, and U.S. Pat. No. 5,877,224.

### Controlled Release Formulations

In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

Matrix devices are a common device for controlling the release of various agents. In such devices, the agents described herein are generally present as a dispersion within the polymer matrix, and are typically formed by the compression of a polymer/drug mixture or by dissolution or melting. The dosage release properties of these devices may be dependent upon the solubility of the agent in the polymer matrix or, in the case of porous matrices, the solubility in the sink solution within the pore network, and the tortuosity of the network. In one instance, when utilizing an erodible polymeric matrix, the matrix imbibes water and forms an aqueous-swollen gel that entraps the agent. The matrix then gradually erodes, swells, disintegrates or dissolves in the GI tract, thereby controlling release of one or more of the agents described herein. In non-erodible devices, the agent is released by diffusion through an inert matrix.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use.

The erodible polymeric matrix into which an agent described herein can be incorporated may generally be described as a set of excipients that are mixed with the agent following its formation that, when contacted with the aqueous environment of use imbibes water and forms a water-swollen gel or matrix that entraps the drug form. Drug release may occur by a variety of mechanisms, for example, the matrix may disintegrate or dissolve from around particles or granules of the agent or the agent may dissolve in the imbibed aqueous solution and diffuse from the tablet, beads or granules of the device. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series.

The choice of matrix material can have a large effect on the maximum drug concentration attained by the device as well as the maintenance of a high drug concentration. The matrix material can be a concentration-enhancing polymer, for example, as described in WO05/011634.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

The erodible matrix polymer may contain a wide variety of the same types of additives and excipients known in the pharmaceutical arts, including osmopolymers, osmagens, solubility-enhancing or -retarding agents and excipients that promote stability or processing of the device.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g. ethyl cellulose, cellulose acetate, crosslinked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000).

Matrix controlled release devices may be prepared by blending an agent described herein and other excipients together, and then forming the blend into a tablet, caplet, pill, or other device formed by compressive forces. Such compressed devices may be formed using any of a wide variety of presses used in the fabrication of pharmaceutical devices. Examples include single-punch presses, rotary tablet presses, and multilayer rotary tablet presses, all well known in the art. See for example, Remington: The Science and Practice of Pharmacy, 20th Edition, 2000. The compressed device may be of any shape, including round, oval, oblong, cylindrical, or triangular. The upper and lower surfaces of the compressed device may be flat, round, concave, or convex.

In certain embodiments, when formed by compression, the device has a strength of at least 5 Kiloponds (Kp)/cm² (for example, at least 7 Kp/cm²). Strength is the fracture force, also known as the tablet hardness required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kP/cm². Friability is a well-know measure of a device's resistance to surface abrasion that measures weight loss in percentage after subjecting the device to a standardized agitation procedure. Friability values of from 0.8 to 1.0% are regarded as constituting the upper limit of acceptability. Devices having a strength of greater than 5 kP/cm² generally are very robust, having a friability of less than 0.5%. Other methods for forming matrix controlled-release devices are well known in the pharmaceutical arts. See for example, Remington: The Science and Practice of Pharmacy, 20th Edition, 2000.

As noted above, the agents described herein may also be incorporated into an osmotic control device. Such devices generally include a core containing one or more agents as described herein and a water permeable, non-dissolving and non-eroding coating surrounding the core which controls the influx of water into the core from an aqueous environment of use so as to cause drug release by extrusion of some or all of the core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Nonlimiting examples of osmotic control devices are disclosed in U.S. patent application Ser. No. 09/495,061.

Osmotic agents create a driving force for transport of water from the environment of use into the core of the device. Osmotic agents include but are not limited to water-swellable hydrophilic polymers, and osmogens (or osmagens). Thus, the core may include water-swellable hydrophilic polymers, both ionic and nonionic, often referred to as osmopolymers and hydrogels. The amount of water-swellable hydrophilic polymers present in the core may range from about 5 to about 80 wt % (including for example, 10 to 50 wt %). Nonlimiting examples of core materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolat. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The core may include a wide variety of additives and excipients that enhance the performance of the dosage form or that promote stability, tableting or processing. Such additives and excipients include tableting aids, surfactants, water-soluble polymers, pH modifiers, fillers, binders, pigments, disintegrants, antioxidants, lubricants and flavorants. Nonlimiting examples of additives and excipients include but are not limited to those described elsewhere herein as well as microcrystalline cellulose, metallic salts of acids (e.g. aluminum stearate, calcium stearate, magnesium stearate, sodium stearate, zinc stearate), pH control agents (e.g. buffers, organic acids, organic acid salts, organic and inorganic bases), fatty acids, hydrocarbons and fatty alcohols (e.g. stearic acid, palmitic acid, liquid paraffin, stearyl alcohol, and palmitol), fatty acid esters (e.g. glyceryl (mono- and di-) stearates, triglycerides, glyceryl (palmiticstearic) ester, sorbitan esters (e.g. sorbitan monostearate, saccharose monostearate, saccharose monopalmitate, sodium stearyl fumarate), polyoxyethylene sorbitan esters), surfactants (e.g. alkyl sulfates (e.g. sodium lauryl sulfate, magnesium lauryl sulfate), polymers (e.g. polyethylene glycols, polyoxyethylene glycols, polyoxyethylene, polyoxypropylene ethers, including copolymers thereof), polytetrafluoroethylene), and inorganic materials (e.g. talc, calcium phosphate), cyclodextrins, sugars (e.g. lactose, xylitol), sodium starch glycolate). Nonlimiting examples of disintegrants are sodium starch glycolate (e.g., Explotab™ CLV, (microcrystalline cellulose (e.g., Avicel™), microcrystalline silicified cellulose (e.g., ProSolv™), croscarmellose sodium (e.g., Ac-Di-Sol™). When the agent described herein is a solid amorphous dispersion formed by a solvent process, such additives may be added directly to the spray-drying solution when forming an agent described herein/concentration-enhancing polymer dispersion such that the additive is dissolved or suspended in the solution as a slurry, Alternatively, such additives may be added following the spray-drying process to aid in forming the final controlled release device.

A nonlimiting example of an osmotic device consists of one or more drug layers containing an agent described herein, such as a solid amorphous drug/polymer dispersion, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic delivery devices may be fabricated in various geometries including bilayer (wherein the core comprises a drug layer and a sweller layer adjacent to each other), trilayer (wherein the core comprises a sweller layer sandwiched between two drug layers) and concentric (wherein the core comprises a central sweller agent surrounded by the drug layer). The coating of such a tablet comprises a membrane permeable to water but substantially impermeable to drug and excipients contained within. The coating contains one or more exit passageways or ports in communication with the drug-containing layer(s) for delivering the drug agent. The drug-containing layer(s) of the core contains the drug agent (including optional osmagents and hydrophilic water-soluble polymers), while the sweller layer consists of an expandable hydrogel, with or without additional osmotic agents.

When placed in an aqueous medium, the tablet imbibes water through the membrane, causing the agent to form a dispensable aqueous agent, and causing the hydrogel layer to expand and push against the drug-containing agent, forcing the agent out of the exit passageway. The agent can swell, aiding in forcing the drug out of the passageway. Drug can be delivered from this type of delivery system either dissolved or dispersed in the agent that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

Other materials useful in forming the drug-containing agent, in addition to the agent described herein itself, include HPMC, PEO and PVP and other pharmaceutically acceptable carriers. In addition, osmagents such as sugars or salts, including but not limited to sucrose, lactose, xylitol, mannitol, or sodium chloride, may be added. Materials which are useful for forming the hydrogel layer include sodium CMC, PEO (e.g. polymers having an average molecular weight from about 5,000,000 to about 7,500,000 daltons), poly(acrylic acid), sodium (polyacrylate), sodium croscarmellose, sodium starch glycolat, PVP, crosslinked PVP, and other high molecular weight hydrophilic materials.

In the case of a bilayer geometry, the delivery port(s) or exit passageway(s) may be located on the side of the tablet containing the drug agent or may be on both sides of the tablet or even on the edge of the tablet so as to connect both the drug layer and the sweller layer with the exterior of the device. The exit passageway(s) may be produced by mechanical means or by laser drilling, or by creating a difficult-to-coat region on the tablet by use of special tooling during tablet compression or by other means.

The osmotic device can also be made with a homogeneous core surrounded by a semipermeable membrane coating, as in U.S. Pat. No. 3,845,770. The agent described herein can be incorporated into a tablet core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above. In one embodiment, an osmotic device comprises: (a) a single-layer compressed core comprising: (i) an agent described herein, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable layer surrounding the core; and (c) at least one passageway within the water-permeable layer (b) for delivering the drug to a fluid environment surrounding the tablet. In certain embodiments, the device is shaped such that the surface area to volume ratio (of a water-swollen tablet) is greater than 0.6 mm⁻¹ (including, for example, greater than 1.0 mm⁻¹). The passageway connecting the core with the fluid environment can be situated along the tablet band area. In certain embodiments, the shape is an oblong shape where the ratio of the tablet tooling axes, i.e., the major and minor axes which define the shape of the tablet, are between 1.3 and 3 (including, for example, between 1.5 and 2.5). In one embodiment, the combination of the agent described herein and the osmagent have an average ductility from about 100 to about 200 Mpa, an average tensile strength from about 0.8 to about 2.0 Mpa, and an average brittle fracture index less than about 0.2. The single-layer core may optionally include a disintegrant, a bioavailability enhancing additive, and/or a pharmaceutically acceptable excipient, carrier or diluent.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™), microcrystalline silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Water-soluble polymers are added to keep particles of the agent suspended inside the device before they can be delivered through the passageway(s) (e.g., an orifice). High viscosity polymers are useful in preventing settling. However, the polymer in combination with the agent is extruded through the passageway(s) under relatively low pressures. At a given extrusion pressure, the extrusion rate typically slows with increased viscosity. Certain polymers in combination with particles of the agent described herein form high viscosity solutions with water but are still capable of being extruded from the tablets with a relatively low force. In contrast, polymers having a low weight-average, molecular weight (<about 300,000) do not form sufficiently viscous solutions inside the tablet core to allow complete delivery due to particle settling. Settling of the particles is a problem when such devices are prepared with no polymer added, which leads to poor drug delivery unless the tablet is constantly agitated to keep the particles from settling inside the core. Settling is also problematic when the particles are large and/or of high density such that the rate of settling increases.

In certain embodiments, the water-soluble polymers for such osmotic devices do not interact with the drug. In certain embodiments the water-soluble polymer is a non-ionic polymer. A nonlimiting example of a non-ionic polymer forming solutions having a high viscosity yet still extrudable at low pressures is Natrosol™ 250H (high molecular weight hydroxyethylcellulose, available from Hercules Incorporated, Aqualon Division, Wilmington, Del.; MW equal to about 1 million daltons and a degree of polymerization equal to about 3,700). Natrosol 250H™ provides effective drug delivery at concentrations as low as about 3% by weight of the core when combined with an osmagent. Natrosol 250H™ NF is a high-viscosity grade nonionic cellulose ether that is soluble in hot or cold water. The viscosity of a 1% solution of Natrosol 250H using a Brookfield LVT (30 rpm) at 25° C. is between about 1, 500 and about 2,500 cps.

In certain embodiments, hydroxyethylcellulose polymers for use in these monolayer osmotic tablets have a weight-average, molecular weight from about 300,000 to about 1.5 million. The hydroxyethylcellulose polymer is typically present in the core in an amount from about 2.0% to about 35% by weight.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

The osmotic controlled release devices of the present invention can also comprise a coating. In certain embodiments, the osmotic controlled release device coating exhibits one or more of the following features: is water-permeable, has at least one port for the delivery of drug, and is non-dissolving and non-eroding during release of the drug formulation, such that drug is substantially entirely delivered through the delivery port(s) or pores as opposed to delivery primarily via permeation through the coating material itself. Delivery ports include any passageway, opening or pore whether made mechanically, by laser drilling, by pore formation either during the coating process or in situ during use or by rupture during use. In certain embodiments, the coating is present in an amount ranging from about 5 to 30 wt % (including, for example, 10 to 20 wt %) relative to the core weight.

One form of coating is a semipermeable polymeric membrane that has the port(s) formed therein either prior to or during use. Thickness of such a polymeric membrane may vary between about 20 and 800 μm (including, for example, between about 100 to 500 μm). The diameter of the delivery port (s) may generally range in size from 0.1 to 3000 μm or greater (including, for example, from about 50 to 3000 μm in diameter). Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. No. 5,612,059 and U.S. Pat. No. 5,698,220. The delivery port may be formed in situ by rupture of the coating, for example, when a collection of beads that may be of essentially identical or of a variable agent are used. Drug is primarily released from such beads following rupture of the coating and, following rupture, such release may be gradual or relatively sudden. When the collection of beads has a variable agent, the agent may be chosen such that the beads rupture at various times following administration, resulting in the overall release of drug being sustained for a desired duration.

Coatings may be dense, microporous or asymmetric, having a dense region supported by a thick porous region such as those disclosed in U.S. Pat. No. 5,612,059 and U.S. Pat. No. 5,698,220. When the coating is dense the coating can be composed of a water-permeable material. When the coating is porous, it may be composed of either a water-permeable or a water-impermeable material. When the coating is composed of a porous water-impermeable material, water permeates through the pores of the coating as either a liquid or a vapor. Nonlimiting examples of osmotic devices that utilize dense coatings include U.S. Pat. No. 3,995,631 and U.S. Pat. No. 3,845,770. Such dense coatings are permeable to the external fluid such as water and may be composed of any of the materials mentioned in these patents as well as other water-permeable polymers known in the art.

The membranes may also be porous as disclosed, for example, in U.S. Pat. No. 5,654,005 and U.S. Pat. No. 5,458,887 or even be formed from water-resistant polymers. U.S. Pat. No. 5,120,548 describes another suitable process for forming coatings from a mixture of a water-insoluble polymer and a leachable water-soluble additive. The porous membranes may also be formed by the addition of pore-formers as disclosed in U.S. Pat. No. 4,612,008. In addition, vapor-permeable coatings may even be formed from extremely hydrophobic materials such as polyethylene or polyvinylidene difluorid that, when dense, are essentially water-impermeable, as long as such coatings are porous. Materials useful in forming the coating include but are not limited to various grades of acrylic, vinyls, ethers, polyamides, polyesters and cellulosic derivatives that are water-permeable and water-insoluble at physiologically relevant pHs, or are susceptible to being rendered water-insoluble by chemical alteration such as by crosslinking Nonlimiting examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxiated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes. In various embodiments, the coating agent comprises a cellulosic polymer, in particular cellulose ethers, cellulose esters and cellulose ester-ethers, i.e., cellulosic derivatives having a mixture of ester and ether substituents, the coating materials are made or derived from poly(acrylic) acids and esters, poly(methacrylic) acids and esters, and copolymers thereof, the coating agent comprises cellulose acetate, the coating comprises a cellulosic polymer and PEG, the coating comprises cellulose acetate and PEG.

Coating is conducted in conventional fashion, typically by dissolving or suspending the coating material in a solvent and then coating by dipping, spray coating or by pan-coating. In certain embodiments, the coating solution contains 5 to 15 wt % polymer. Typical solvents useful with the cellulosic polymers mentioned above include but are not limited to acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described, for example, in U.S. Pat. No. 5,612,059. Coatings may also be hydrophobic microporous layers wherein the pores are substantially filled with a gas and are not wetted by the aqueous medium but are permeable to water vapor, as disclosed, for example, in U.S. Pat. No. 5,798,119. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Hydrophobic microporous coating materials include but are not limited to polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed, for example, in U.S. Pat. No. 4,247,498, U.S. Pat. No. 4,490,431 and U.S. Pat. No. 4,744,906. Osmotic controlled-release devices may be prepared using procedures known in the pharmaceutical arts. See for example, Remington: The Science and Practice of Pharmacy, 20th Edition, 2000.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size. Other excipients, such as a binder (e.g., microcrystalline cellulose), may be blended with the agent to aid in processing and forming the multiparticulates. In the case of wet granulation, a binder such as microcrystalline cellulose may be included in the granulation fluid to aid in forming a suitable multiparticulate. See, for example, Remington: The Science and Practice of Pharmacy, 20″ Edition, 2000. In any case, the resulting particles may themselves constitute the therapeutic composition or they may be coated by various film-forming materials such as enteric polymers or water-swellable or water-soluble polymers, or they may be combined with other excipients or vehicles to aid in dosing to patients.

In certain embodiments, it may be desirable to provide for the immediate release of one or more of the agents described herein, and the controlled release of one or more other agents. For example, in one embodiment, a compound described herein can be provided in an immediate release formulation together with a cotherapy agent described herein in a controlled release format. For example, in one embodiment, a compound described herein can be provided in a controlled release format together with a cotherapy agent described herein in an immediate release format.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds described herein can be incorporated into pharmaceutically-acceptable nanoparticle, nanosphere, and nanocapsule formulations (Delie and Blanco-Prieto 2005 Molecule 10:65-80). Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art (Couvreur et al, 1980; 1988; zur Muhlen et al., 1998; Zambaux et al. 1998; Pinto-Alphandry et al., 1995 and U.S. Pat. No. 5,145,684).

The compounds described herein can be formulated with pH sensitive materials which may include those described in WO04041195 (including the seal and enteric coating described therein) and pH-sensitive coatings that achieve delivery in the colon including those described in U.S. Pat. No. 4,910,021 and WO9001329. U.S. Pat. No. 4,910,021 describes using a pH-sensitive material to coat a capsule. WO9001329 describes using pH-sensitive coatings on beads containing acid, where the acid in the bead core prolongs dissolution of the pH-sensitive coating. U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher. The compounds described herein may be formulated in the pH triggered targeted control release systems described in WO04052339. The compounds described herein may be formulated according to the methodology described in any of WO03105812 (extruded hyrdratable polymers); WO0243767 (enzyme cleavable membrane translocators); WO03007913 and WO03086297 (mucoadhesive systems); WO02072075 (bilayer laminated formulation comprising pH lowering agent and absorption enhancer); WO04064769 (amidated peptides); WO05063157 (solid lipid suspension with pseudotropic and/or thixotropic properties upon melting); WO03035029 and WO03035041 (erodible, gastric retentive dosage forms); U.S. Pat. No. 5,007,790 and U.S. Pat. No. 5,972,389 (sustained release dosage forms); WO04112711 (oral extended release compositions); WO05027878, WO02072033, and WO02072034 (delayed release compositions with natural or synthetic gum); WO05030182 (controlled release formulations with an ascending rate of release); WO05048998 (microencapsulation system); U.S. Pat. No. 5,952,314 (biopolymer); U.S. Pat. No. 5,108,758 (glassy amylose matrix delivery); U.S. Pat. No. 5,840,860 (modified starch based delivery). JP10324642 (delivery system comprising chitosan and gastric resistant material such as wheat gliadin or zein); U.S. Pat. No. 5,866,619 and U.S. Pat. No. 6,368,629 (saccharide containing polymer); U.S. Pat. No. 6,531,152 (describes a drug delivery system containing a water soluble core (Ca pectinate or other water-insoluble polymers) and outer coat which bursts (eg hydrophobic polymer-Eudragrit)); U.S. Pat. No. 6,234,464; U.S. Pat. No. 6,403,130 (coating with polymer containing casein and high methoxy pectin; WO0174175 (Maillard reaction product); WO05063206 (solubility increasing formulation); WO04019872 (transferring fusion proteins). The compounds described herein may be formulated using gastrointestinal retention system technology (GIRES; Merrion Pharmaceuticals). GIRES comprises a controlled-release dosage form inside an inflatable pouch, which is placed in a drug capsule for oral administration. Upon dissolution of the capsule, a gas-generating system inflates the pouch in the stomach where it is retained for 16-24 hours, all the time releasing compounds described herein.

The compounds described hereincan be formulated in an osmotic device including the ones disclosed in U.S. Pat. No. 4,503,030, U.S. Pat. No. 5,609,590 and U.S. Pat. No. 5,358,502. U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption. U.S. Pat. Nos. 5,609,590 and 5,358,502 disclose an osmotic bursting device for dispensing a beneficial agent to an aqueous environment. The device comprises a beneficial agent and osmagent surrounded at least in part by a semi-permeable membrane. The beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

The compounds described hereinmay be formulated based on the invention described in U.S. Pat. No. 5,316,774 which discloses a composition for the controlled release of an active substance comprising a polymeric particle matrix, where each particle defines a network of internal pores. The active substance is entrapped within the pore network together with a blocking agent having physical and chemical characteristics selected to modify the release rate of the active substance from the internal pore network. In one embodiment, drugs may be selectively delivered to the intestines using an enteric material as the blocking agent. The enteric material remains intact in the stomach but degrades under the pH conditions of the intestines. In another embodiment, the sustained release formulation employs a blocking agent, which remains stable under the expected conditions of the environment to which the active substance is to be released. The use of pH-sensitive materials alone to achieve site-specific delivery is difficult because of leaking of the beneficial agent prior to the release site or desired delivery time and it is difficult to achieve long time lags before release of the active ingredient after exposure to high pH (because of rapid dissolution or degradation of the pH-sensitive materials).

The agents may also be formulated in a hybrid system which combines pH-sensitive materials and osmotic delivery systems. These hybrid devices provide delayed initiation of sustained-release of the beneficial agent. In one device a pH-sensitive matrix or coating dissolves releasing osmotic devices that provide sustained release of the beneficial agent see U.S. Pat. Nos. 4,578,075, 4,681,583, and 4,851,231. A second device consists of a semipermeable coating made of a polymer blend of an insoluble and a pH-sensitive material. As the pH increases, the permeability of the coating increases, increasing the rate of release of beneficial agent see U.S. Pat. Nos. 4,096,238, 4,503,030, 4,522,625, and 4,587,117.

The compounds described herein may be formulated in terpolumers according to U.S. Pat. No. 5,484,610 which discloses terpolymers which are sensitive to pH and temperature which are useful carriers for conducting bioactive agents through the gastric juices of the stomach in a protected form. The terpolymers swell at the higher physiologic pH of the intestinal tract causing release of the bioactive agents into the intestine. The terpolymers are linear and are made up of 35 to 99 wt % of a temperature sensitive component, which imparts to the terpolymer LCST (lower critical solution temperature) properties below body temperatures, 1 to 30 wt % of a pH sensitive component having a pKa in the range of from 2 to 8 which functions through ionization or deionization of carboxylic acid groups to prevent the bioactive agent from being lost at low pH but allows bioactive agent release at physiological pH of about 7.4 and a hydrophobic component which stabilizes the LCST below body temperatures and compensates for bioactive agent effects on the terpolymers. The terpolymers provide for safe bioactive agent loading, a simple procedure for dosage form fabrication and the terpolymer functions as a protective carrier in the acidic environment of the stomach and also protects the bioactive agents from digestive enzymes until the bioactive agent is released in the intestinal tract.

The compounds described herein may be formulated in pH sensitive polymers according to those described in U.S. Pat. No. 6,103,865. U.S. Pat. No. 6,103,865 discloses pH-sensitive polymers containing sulfonamide groups, which can be changed in physical properties, such as swellability and solubility, depending on pH and which can be applied for a drug-delivery system, bio-material, sensor, and the like, and a preparation method therefore. The pH-sensitive polymers are prepared by introduction of sulfonamide groups, various in pKa, to hydrophilic groups of polymers either through coupling to the hydrophilic groups of polymers, such as acrylamide, N,N-dimethylacrylamide, acrylic acid, N-isopropylacrylamide and the like or copolymerization with other polymerizable monomers. These pH-sensitive polymers may have a structure of linear polymer, grafted copolymer, hydrogel or interpenetrating network polymer.

The compounds described herein may be formulated according U.S. Pat. No. 5,656,292 which discloses a composition for pH dependent or pH regulated controlled release of active ingredients especially drugs. The composition consists of a compactable mixture of the active ingredient and starch molecules substituted with acetate and dicarboxylate residues. The preferred dicarboxylate acid is succinate. The average substitution degree of the acetate residue is at least 1 and 0.2-1.2 for the dicarboxylate residue. The starch molecules can have the acetate and dicarboxylate residues attached to the same starch molecule backbone or attached to separate starch molecule backbones. The present invention also discloses methods for preparing said starch acetate dicarboxylates by transesterification or mixing of starch acetates and starch dicarboxylates respectively.

The compounds described herein may be formulated according to the methods described in U.S. Pat. Nos. 5,554,147,5, 788, 687, and 6,306,422 which disclose a method for the controlled release of a biologically active agent wherein the agent is released from a hydrophobic, pH-sensitive polymer matrix. The polymer matrix swells when the environment reaches pH 8.5, releasing the active agent. A polymer of hydrophobic and weakly acidic comonomers is disclosed for use in the controlled release system. Also disclosed is a specific embodiment in which the controlled release system may be used. The pH-sensitive polymer is coated onto a latex catheter used in ureteral catheterization. A ureteral catheter coated with a pH-sensitive polymer having an antibiotic or urease inhibitor trapped within its matrix will release the active agent when exposed to high pH urine.

The compounds described herein may be formulated in/with bioadhesive polymers according to U.S. Pat. No. 6,365,187. Bioadhesive polymers in the form of, or as a coating on, microcapsules containing drugs or bioactive substances which may serve for therapeutic, or diagnostic purposes in diseases of the gastrointestinal tract, are described in U.S. Pat. No. 6,365,187. The polymeric microspheres all have a bioadhesive force of at least 11 mN/cm² (110 N/m2) Techniques for the fabrication of bioadhesive microspheres, as well as a method for measuring bioadhesive forces between microspheres and selected segments of the gastrointestinal tract in vitro are also described. This quantitative method provides a means to establish a correlation between the chemical nature, the surface morphology and the dimensions of drug-loaded microspheres on one hand and bioadhesive forces on the other, allowing the screening of the most promising materials from a relatively large group of natural and synthetic polymers which, from theoretical consideration, should be used for making bioadhesive microspheres. Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be fused to immunoglobulins or albumin, or incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

Methods to increase chemical and/or physical stability of the agents the described herein are found in WO 00/04880, and WO 97/04796 and the references cited therein.

Methods to increase bioavailability of the agents described herein are found in U.S. 20030198619, WO 01/49268, WO 00/32172, and WO 02/064166. Glycyrrhizinate can also be used as an absorption enhancer (see, e.g., EP397447). WO 03/004062 discusses Ulex europaeus I (UEAl) and UEAI mimetics which may be used to target the agents to the GI tract.

Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for cotherapy use, a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

### EXAMPLES

### Scheme 1: Preparation of ethyl 2-(2,5,5-trimethyl-4,5,6,7-tetrahydro-1H-indol-1-yl)acetate (3)

Scheme 1A: Preparation of 4,4-Dimethyl-2-(2-oxopropyl)cyclohexanone (2). Sodium hydride (2.61 g, 65.4 mmol, 60% dispersion in mineral oil) was diluted in anhydrous toluene (20 mL) and stirred for 5 min, then allowed to settle, after which the supernatant was removed via cannula. The slurry was rediluted in toluene (30 mL) followed by the addition of a solution of 4,4-dimethylcyclohexanone (1) (7.50 g, 59.4 mmol) in toluene (30 mL). The reaction was heated to 120° C. for 3 h, after which neat 3-chloro-2-(methoxymethoxy)prop-1-ene (8.93 g, 65.4 mmol) was added. After heating to reflux for an additional 12 h, the reaction was cooled to room temperature, quenched by the addition of methanol (3 mL), extracted with dichloromethane (3×50 mL), dried (sodium sulfate), filtered, and concentrated to a brown residue which was reconstituted in dioxane (20 mL) and water (20 mL). Sulfuric acid (0.12 mL, 2.2 mmol) was added, and the resulting solution was refluxed for 2.5 h at 100° C. then cooled to room temperature, extracted with dichloromethane (3×100 mL), dried (sodium sulfate), filtered, and concentrated to a brown oil. Purification by silica gel chromatography (Luknova 120 g, 20 mL/min) using 10 to 70% ethyl acetate in hexanes (over 40 min) afforded the product, 4,4-dimethyl-2-(2-oxopropyl)cyclohexanone (2) (2.5 g, 13.8 mmol). Later fractions were also collected, and were repurified using the same chromatography conditions, but using a Luknova 80 g silica column with a 20 mL/min flow rate. From this second purification, additional 4,4-dimethyl-2-(2-oxopropyl)cyclohexanone (1.70 g, 9.30 mmol) was isolated as a gold oil (total yield 4.2 g, 23.1 mmol, 39%). ¹H NMR (400 MHz, CDCl₃) δ (ppm): 3.06-3.15 (m, 1H), 2.91 (dd, 1H, J=17.6 Hz, J=7.6 Hz), 2.52 (app. td, 1H, J=14.4 Hz, J=6.0 Hz), 2.24 (m, 1H), 2.20 (s, 3H), 2.04-2.10 (m, 1H), 1.67-1.75 (m, 2H), 1.58-1.64 (m, 1H), 1.36 (dd, 1H, J=13.6 Hz, J=13.2 Hz), 1.24 (s, 3H), 0.99 (s, 3H).

Scheme 1B: Preparation of Ethyl 2-(2,5,5-trimethyl-4,5,6,7-tetrahydro-1H-indol-1-yl)acetate (3). Ethyl 2-aminoacetate hydrochloride (1.53 g, 11.0 mmol) was added to a room temperature solution of 4,4-dimethyl-2-(2-oxopropyl)cyclohexanone (2) (2.00 g, 11.0 mmol) in dichloromethane (27.4 mL). To this was added solid sodium bicarbonate (2.305 g, 27.4 mmol), and the reaction was stirred at 55° C. for 15 h (overnight). The reaction was then cooled to room temperature, extracted with dichloromethane (3×40 mL), dried (sodium sulfate), filtered and concentrated. Purification by silica gel chromatography (Luknova 80 g, 20 mL/min) using 0 to 70% ethyl acetate in hexanes (over 40 min) afforded the product, ethyl 2-(2,5,5-trimethyl-4,5,6,7-tetrahydro-1H-indol-1-yl)acetate (3) (2.09 g, 8.38 mmol, 76%), as a clear colorless oil. Starting material, 4,4-dimethyl-2-(2-oxopropyl)cyclohexanone (2) (0.111 g, 0.609 mmol, 5.55%), was also recovered as a clear, colorless oil. ¹H NMR (400 MHz, CDCl₃) δ (ppm): 5.71 (s, 1H), 4.47 (s, 2H), 4.24 (q, 2H, J=6.8 Hz), 2.45 (t, 2H, J=6.4 Hz), 2.30 (s, 2H), 2.20 (s, 3H), 1.59 (t, 2H, J=6.4 Hz), 1.30 (t, 3H, J=6.8 Hz), 1.00 (s, 6H).

### Scheme 2: General Synthesis for Functionalized Dimethyl Tetrahydroindoles (4) and (5)

### Example 1

Preparation of Ethyl 2-(2,5,5-trimethyl-3-(2-(morpholinosulfonyl)benzyl)-4,5,6,7-tetrahydro-1H-indol-1-yl)acetate (I-25) To a −10° C. solution of trimethylsilyl trifluoromethanesulfonate (2.15 mL, 11.9 mmol) in dichloromethane (100 mL) was added a solution of ethyl 2-(2,5,5-trimethyl-4,5,6,7-tetrahydro-1H-indol-1-yl)acetate (3) (1.48 g, 5.94 mmol) and 2-(morpholinosulfonyl)benzaldehyde (1.89 g, 7.12 mmol) in dichloromethane (30 mL). The reaction was allowed to stir at −10° C. for 15 min, after which neat triethylsilane (2.84 mL, 17.81 mmol) was added to the reaction. The reaction was stirred for 3 h at −10° C. (internal temperature), after which the reaction was slowly warmed to room temperature (over 1 hr). An additional 0.10 equivalent of aldehyde (188 mg, 0.736 mmol) was added, and the reaction was continued stirring for another 45 min at room temperature, then stored in a freezer overnight. Most of (3) had disappeared. The reaction (now a total time of 20 h) was quenched at 0° C. by the addition of saturated sodium bicarbonate solution (50 mL). Extraction with dichloromethane (3×40 mL), drying (sodium sulfate), then filtration furnished a crude brown residue which was purified by silica gel chromatography (Luknova 120 g, 20 mL/min) using 1 to 5% (7/1 MeCN/MeOH) in dichloromethane over 50 min affording ethyl 2-(2,5,5-trimethyl-3-(2-(morpholinosulfonyl)benzyl)-4,5,6,7-tetrahydro-1H-indol-1-yl)acetate (I-25) (820 mg, 1.68 mmol). An additional 182 mg of desired product was also recovered with ˜95% purity, which was later repurified using the same chromatography conditions leading to a total of 1.00 g isolated of the title compound (35%). The byproducts diethyl 2,2′-(3,3′-((2 (morpholinosulfonyl)phenyl)methylene)bis(2,5,5-trimethyl-4,5,6,7-tetrahydro-1H-indole-3,1-diyl))diacetate (280 mg, 0.342 mmol, 6%) and (2-(morpholinosulfonyl)phenyl)methanol (890 mg, 3.46 mmol, 45%) were also isolated as a dark red residue and a tan solid, respectively. ¹H NMR (400 MHz, CDCl₃) δ (ppm): 7.96 (d, 1H, J=7.6 Hz), 7.40 (t, 1H, J=7.2 Hz), 7.28 (t, 1H, J=7.6 Hz), 7.08 (d, 1H, J=7.2 Hz), 4.49 (s, 2H), 4.22 (q, 2H, J=6.8 Hz), 4.12 (s, 2H), 3.75 (m, 4H), 3.24 (m, 4H), 2.45 (t, 2H, J=6.0 Hz), 2.02 (s, 3H), 1.98 (s, 2H), 1.56 (t, 2H, J=6.0 Hz), 1.29 (t, 3H, J=7.2 Hz), 0.91 (s, 6H).

### Example 2

Preparation of 2-(2,5,5-Trimethyl-3-(2-(morpholinosulfonyl)benzyl)-4,5,6,7-tetrahydro-1H-indol-1-yl)acetic acid (I-17). To a solution of ethyl 2-(2,5,5-trimethyl-3-(2-(morpholinosulfonyl)benzyl)-4,5,6,7-tetrahydro-1H-indol-1-yl)acetate (I-25) (805 mg, 1.65 mmol) in THF (2.0 mL) and water (2.0 mL) at 0° C. was added 1N aqueous NaOH solution (3.30 mL, 3.30 mmol). The reaction was warmed to room temperature and stirred for 2.5 h after which the reaction was complete. The reaction mixture was then cooled to 0° C. then quenched by the addition of 3N aqueous HCl solution (1.10 mL), then concentrated to remove all the THF leading to the formation of a brown residue slick on the surface of the remaining water phase. Dichloromethane (25 mL) was added, and the reaction was extracted with dichloromethane (3×25 mL), dried (sodium sulfate), filtered, and concentrated to 2-(2,5,5-trimethyl-3-(2-(morpholinosulfonyl)benzyl)-4,5,6,7-tetrahydro-1H-indol-1-yl)acetic acid (I-17) (733 mg, 1.60 mmol, 97%), a blue-green solid. ¹H NMR (400 MHz, CDCl₃) δ (ppm): 7.96 (d, 1H, J=8.0 Hz), 7.40 (t, 1H, J=7.6 Hz), 7.29 (t, 1H, J=7.6 Hz), 7.05 (d, 1H, J=7.6 Hz), 4.56 (s, 2H), 4.13 (s, 2H), 3.74 (m, 4H), 3.24 (m, 4H), 2.45 (t, 2H, J=5.6 Hz), 2.04 (s, 3H), 1.99 (s, 2H), 1.57 (t, 2H, J=6.4 Hz), 0.90 (s, 6H).

### Example 3

Preparation of Ethyl 2-(2,5,5-trimethyl-3-(2-(phenylsulfonyl)benzyl)-4,5,6,7-tetrahydro-1H-indol-1-yl)acetate (I-28) To a flame-dried vial purged with N₂ was added dichloromethane (2 mL) followed by trimethylsilyl trifluoromethanesulfonate (0.145 mL, 0.802 mmol). After cooling to 0° C., a solution of 2-(phenylsulfonyl)benzaldehyde (99.0 mg, 0.401 mmol) and ethyl 2-(2,5,5-trimethyl-4,5,6,7-tetrahydro-1H-indol-1-yl)acetate (3) (100 mg, 0.401 mmol) in dichloromethane (5 mL) was added. The solution was stirred for 30 min at 0° C., after which neat triethylsilane (0.256 mL, 1.60 mmol) was added. The reaction was continued stirring at 0° C. for 30 min, after which it was quenched at 0° C. by the addition of saturated sodium bicarbonate solution (20 mL), extracted with dichloromethane (3×30 mL), dried (sodium sulfate), filtered, and concentrated to a dark residue which was purified on silica gel using 0 to 70% ethyl acetate in hexanes (over 50 min) then 80% ethyl acetate (with 1% NEt₃) in hexanes for another 30 min. Two products were obtained, The first of which was the desired product, ethyl 2-(2,5,5-trimethyl-3-(2-(phenylsulfonyl)benzyl)-4,5,6,7-tetrahydro-1H-indol-1-yl)acetate (I-28) (67.4 mg, 0.141 mmol, 35.0%), the second is diethyl 2,2′-(3,3′-((2-(phenylsulfonyl)phenyl)methylene)bis(2,5,5-trimethyl-4,5,6,7-tetrahydro-1H-indole-3,1-diyl))diacetate (55.2 mg, 0.0760 mmol, 19%). ¹H NMR (400 MHz, CDCl₃) δ (ppm): 8.30 (dd, 1H, J=7.6 Hz, J=1.6 Hz), 7.90 (m, 2H), 7.57 (m, 1H), 7.50 (m, 2H), 7.35-7.47 (m, 2H), 6.97 (dd, 1H, J=7.6 Hz, J=0.8 Hz), 4.42 (s, 2H), 4.19 (q, 2H, J=7.2 Hz), 3.3 (s, 2H), 2.37 (t, 2H, J=6.0 Hz), 1.81 (s, 3H), 1.45 (m, 4H, two shifts isochronous), 1.26 (t, 3H, J=6.8 Hz), 0.76 (s, 6H).

### Example 4

Preparation of 2-(2,5,5-Trimethyl-3-(2-(phenylsulfonyl)benzyl)-4,5,6,7-tetrahydro-1H-indol-1-yl)acetic acid (I-9). To a room temperature solution of ethyl 2-(2,5,5-trimethyl-3-(2-(phenylsulfonyl)benzyl)-4,5,6,7-tetrahydro-1H-indol-1-yl)acetate (I-28) (67.4 mg, 0.141 mmol) in THF (3.0 mL), MeOH (1.0 mL), and water (1.0 mL), was added solid lithium hydroxide monohydrate (23.6 mg, 0.562 mmol). The reaction was stirred at room temperature for 1 h after which the reaction was quenched at room temperature by the addition of 3N HCl (0.187 mL) then concentrated to about 20% of its total volume. The resulting brown precipitate was filtered and washed with water (10 mL) then dried to afford 2-(2,5,5-trimethyl-3-(2-(phenylsulfonyl)benzyl)-4,5,6,7-tetrahydro-1H-indol-1-yl)acetic acid (I-9) (49.7 mg, 0.110 mmol, 78%) as a tan solid. ¹H NMR (400 MHz, CDCl₃) δ (ppm): 8.23 (dd, 1H, J=7.6 Hz, J=1.6 Hz), 7.82 (m, 2H), 7.51 (m, 1H), 7.41 (m, 2H), 7.35 (app. td, 1H, J=7.6 Hz, J=2.0 Hz), 7.31 (m, 1H), 6.88 (d, 1H, J=6.8 Hz), 4.43 (s, 2H), 3.76 (s, 2H), 2.31 (t, 2H, J=6.0 Hz), 1.77 (s, 3H), 1.39 (m, 4H, two shifts isochronous), 0.70 (s, 6H).

### Example 5

Preparation of Ethyl 2-(2,5,5-trimethyl-3-(2-(pyrrolidin-1-ylsulfonyl)benzyl)-4,5,6,7-tetrahydro-1H-indol-1-yl)acetate (I-29). To a solution of ethyl 2-(2,5,5-trimethyl-4,5,6,7-tetrahydro-1H-indol-1-yl)acetate (3) (123 mg, 0.494 mmol) in dichloromethane (19 mL) at 0° C. was added neat trimethylsilyl trifluoromethanesulfonate (0.089 mL, 0.494 mmol) followed by neat triethylsilane (0.158 mL, 0.987 mmol). A solution of 2-(pyrrolidin-1-ylsulfonyl)benzaldehyde (125 mg, 0.522 mmol) in dichloromethane (8 mL) was slowly added to the reaction (over 4 min). After 1.2 h, an additional 0.37 equiv of 2-(pyrrolidin-1-ylsulfonyl)benzaldehyde was added (62.4 mg, 0.261 mmol) in dichloromethane (4.5 mL), and the reaction was continued stirring at 0° C. After 2 h, an additional 0.37 equivalents of 2-(pyrrolidin-1-ylsulfonyl)benzaldehyde was added (62.4 mg, 0.251 mmol) in dichloromethane 8.0 mL), and the reaction was slowly allowed to reach room temperature. The reaction was stopped after 3 h by the addition of saturated sodium bicarbonate solution (40 mL), then extracted with dichloromethane (3×40 mL), dried (sodium sulfate), filtered, and concentrated to a salmon-colored residue which was purified by silica gel chromatography (Luknova 80 g, 20 mL/min) with 7/1 MeCN/MeOH in dichloromethane (1% for 8 min then gradient to 7% over 35 min), affording ethyl 2-(2,5,5-trimethyl-3-(2-(pyrrolidin-1-ylsulfonyl)benzyl)-4,5,6,7-tetrahydro-1H-indol-1-yl)acetate (I-29) (81.3 mg, 0.172 mmol, 34.8%) as a clear, colorless residue. ¹H NMR (400 MHz, CDCl₃) δ (ppm): 7.95 (d, 1H, J=8.0 Hz), 7.36 (t, 1H, J=8.0 Hz), 7.26 (m, 1H), 7.06 (d, 1H, J=7.6 Hz), 4.49 (s, 2H), 4.22 (q, 2H, J=7.6 Hz), 4.12 (s, 2H), 3.36 (m, 4H), 2.45 (m, 2H), 2.01 (s, 3H), 1.94 (m, 6H, two shifts isochronous), 1.55 (t, 2H, J=6.0 Hz), 1.28 (t, 3H, J=7.2 Hz), 0.89 (s, 6H).

### Example 6

Preparation of 2-(2,5,5-Trimethyl-3-(2-(pyrrolidin-1-ylsulfonyl)benzyl)-4,5,6,7-tetrahydro-1H-indol-1-yl)acetic acid (I-14). To a 0° C. solution of ethyl 2-(2,5,5-trimethyl-3-(2-(pyrrolidin-1-ylsulfonyl)benzyl)-4,5,6,7-tetrahydro-1H-indol-1-yl)acetate) (I-29) (473 mg, 1.00 mmol) in THF (13.5 mL) and water (13.5 mL) was added a 1N solution of sodium hydroxide (2.00 mL, 2.00 mmol). The ice bath was removed and the reaction was stirred at room temperature for 2 h, after which the reaction was recooled to 0° C. and quenched by the addition of 3N HCl (0.667 mL), then concentrated to about 30% of its original volume. The reaction was extracted with dichloromethane (3×30 mL), dried (sodium sulfate), filtered and concentrated to afford 2-(2,5,5-trimethyl-3-(2-(pyrrolidin-1-ylsulfonyl)benzyl)-4,5,6,7-tetrahydro-1H-indol-1-yl)acetic acid (I-14) (333 mg, 0.748 mmol, 74.7%) which was isolated as a greenish-white solid. ¹H NMR (400 MHz, CDCl₃) δ (ppm): 7.96 (dd, 1H, J=7.6 Hz, J=1.2 Hz), 7.37 (ddd, 1H, J=7.6 Hz, J=7.2 Hz, J=0.8 Hz), 7.26 (m, 1H), 7.03 (d, 1H, J=7.6 Hz), 4.56 (s, 2H), 4.13 (s, 2H), 3.36 (t, 4H, J=6.4 Hz), 2.46 (t, 2H, J=6.0 Hz), 2.04 (s, 3H), 1.98 (s, 2H), 1.93 (m, 4H), 1.56 (t, 2H, J=6.4 Hz), 0.90 (s, 6H).

### Example 7

Preparation of Ethyl 2-(2,5,5-trimethyl-3-(4-(pyrrolidin-1-ylsulfonyl)benzyl)-4,5,6,7-tetrahydro-1H-indol-1-yl)acetate (I-30). To a flame-dried vial was charged with dichloromethane (2 mL) and trimethylsilyl trifluoromethanesulfonate (0.06 mL, 0.32 mmol) was added at 0° C., followed by a solution of ethyl 2-(2,5,5-trimethyl-4,5,6,7-tetrahydro-1H-indol-1-yl)acetate (40 mg, 0.16 mmol) (3) and 4-(pyrrolidin-1-ylsulfonyl)benzaldehyde (38.4 mg, 0.16 mmol) in dichloromethane (5 mL) (cannulated), the mixture was stirred at 0° C. for 15 min, neat triethylsilane (0.10 mL, 0.64 mmol) was added slowly, stirred for 30 min at 0° C., then slowly warmed to room temperature. TLC analysis showed that the reaction was complete, quenched with sat. NaHCO₃, extracted with dichloromethane. Combined extracts were dried with Na₂SO₄, filtered and concentrated, the residue was purified via prep-HPLC to give ethyl 2-(2,5,5-trimethyl-3-(4-(pyrrolidin-1-ylsulfonyl)benzyl)-4,5,6,7-tetrahydro-1H-indol-1-yl)acetate (I-30) 15 mg, 0.03 mmol, 19.8%, as a blue solid. ¹H NMR (400 MHz, CDCl₃) δ (ppm): 7.68 (d, 2H, J=8.0 Hz), 7.26 (d, 2H, J=8.4 Hz), 4.47 (s, 2H), 4.21 (q, 2H, J=7.2 Hz), 3.77 (s, 2H), 3.23 (m, 4H), 2.42 (m, 2H), 2.09 (s, 3H), 2.01 (s, 2H), 1.73 (m, 4H), 1.53 (t, 2H, J=6.8 Hz), 1.28 (t, 3H, J=7.2 Hz), 0.90 (s, 6H).

### Example 8

Preparation of 2-(2,5,5-Trimethyl-3-(4-(pyrrolidin-1-ylsulfonyl)benzyl)-4,5,6,7-tetrahydro-1H-indol-1-yl)acetic acid (I-4) Ethyl 2-(2,5,5-trimethyl-3-(4-(pyrrolidin-1-ylsulfonyl)benzyl)-4,5,6,7-tetrahydro-1H-indol-1-yl)acetate (I-30) (210 mg, 0.44 mmol) was dissolved with stirring in THF (4 mL), MeOH (1 mL) and water (1 mL) LiOH (21.28 mg, 0.88 mmol) was added and the reaction was stirred at 25° C. for 2 h, TLC showed disappearance of starting material. The solvent was removed, water was added, followed by 1 N HCl, remove solvent, MeOH was added, purified by prep-HPLC to give 2-(2,5,5-trimethyl-3-(4-(pyrrolidin-1-ylsulfonyl)benzyl)-4,5,6,7-tetrahydro-1H-indol-1-yl)acetic acid (I-4) (110 mg, 0.25 mmol), light blue solid, 55.7%. ¹H NMR (400 MHz, CDCl₃) δ (ppm): 7.69 (d, 2H, J=8.4 Hz), 7.25 (d, 2H, J=8.0 Hz), 4.54 (s, 2H), 3.78 (s, 2H), 3.22 (m, 4H), 2.42 (m, 2H), 2.11 (s, 3H), 2.02 (s, 2H), 1.73 (m, 4H), 1.53 (m, 2H), 0.91 (s, 6H).

The compounds of Examples 9 to 14 synthesized via general route for compounds I-4 using ethyl 2-(2,5,5-trimethyl-4,5,6,7-tetrahydro-1H-indol-1-yl)acetate (3) as the starting material.

### Example 9

2-(2,5,5-Trimethyl-3-(4-(morpholinosulfonyl)benzyl)-4,5,6,7-tetrahydro-1H-indol-1-yl)acetic acid (I-11) ¹H NMR (400 MHz, CDCl₃) δ (ppm): 7.61 (d, 2H, J=8.4 Hz), 7.27 (d, 2H, J=8.4 Hz), 4.54 (s, 2H), 3.78 (s, 2H), 3.73 (t, 4H, J=4.8 Hz), 2.97 (t, 4H, J=4.8 Hz), 2.43 (t, 2H, J=6.0 Hz), 2.10 (s, 3H), 2.04 (s, 2H), 1.55 (t, 2H, J=6.4 Hz), 0.92 (s, 6H).

### Example 10

2-(3-(4-(4-(Ethoxycarbonyl)piperazin-1-ylsulfonyl)benzyl)-2,5,5-trimethyl-4,5,6,7-tetrahydro-1H-indol-1-yl)acetic acid (I-12) ¹H NMR (400 MHz, CDCl₃) δ (ppm): 7.59 (d, 2H, J=8.0 Hz), 7.27 (d, 2H, J=6.8 Hz), 4.53 (s, 2H), 4.08 (q, 2H, J=7.2 Hz), 3.78 (s, 2H), 3.55 (m, 4H), 2.95 (m, 4H), 2.43 (t, 2H, J=6.0 Hz), 2.10 (s, 3H), 2.04 (s, 2H), 1.54 (t, 2H, J=6.8 Hz), 1.21 (t, 3H, J=7.2 Hz), 0.91 (s, 6H).

### Example 11

2-(2,5,5-Trimethyl-3-(4-(piperidin-1-ylsulfonyl)benzyl)-4,5,6,7-tetrahydro-1H-indol-1-yl)acetic acid (I-13). ¹H NMR (400 MHz, CDCl₃) δ (ppm): 7.61 (d, 2H, J=8.0 Hz), 7.25 (d, 2H, J=7.6 Hz), 4.54 (s, 2H), 3.78 (s, 2H), 2.95 (t, 4H, J=5.2 Hz), 2.43 (t, 2H, J=6.0 Hz), 2.11 (s, 3H), 2.03 (s, 2H), 1.63 (m, 4H), 1.54 (t, 2H, J=6.4 Hz), 1.41 (m, 2H), 0.91 (s, 6H).

### Example 12

2-(3-(4-(N-Cyclopropylsulfamoyl)benzyl)-2,5,5-trimethyl-4,5,6,7-tetrahydro-1H-indol-1-yl)acetic acid (I-15). ¹H NMR (400 MHz, CDCl₃) δ (ppm): 7.76 (d, 2H, J=8.4 Hz), 7.29 (d, 2H, J=8.4 Hz), 4.98 (br, 1H), 4.54 (s, 2H), 3.78 (s, 2H), 2.43 (t, 2H, J=5.6 Hz), 2.23 (m, 1H), 2.11 (s, 3H), 2.02 (s, 2H), 1.54 (t, 2H, J=6.4 Hz), 0.91 (s, 6H), 0.54-0.61 (m, 4H).

### Example 13

2-(3-(4-(Azepan-1-ylsulfonyl)benzyl)-2,5,5-trimethyl-4,5,6,7-tetrahydro-1H-indol-1-yl)acetic acid (I-16. ¹H NMR (400 MHz, CDCl₃) δ (ppm): 7.65 (d, 2H, J=8.4 Hz), 7.22 (d, 2H, J=8.0 Hz), 4.54 (s, 2H), 3.76 (s, 2H), 3.24 (t, 4H, J=6.0 Hz), 2.43 (t, 2H, J=6.0 Hz), 2.10 (s, 3H), 2.03 (s, 2H), 1.70 (m, 4H), 1.52-1.59 (m, 6H), 0.91 (s, 6H).

### Example 14

2-(3-(4-(N,N-Dimethylsulfamoyl)benzyl)-2,5,5-trimethyl-4,5,6,7-tetrahydro-1H-indol-1-yl)acetic acid (I-18). ¹H NMR (400 MHz, CDCl₃) δ (ppm): 7.64 (d, 2H, J=8.4 Hz), 7.27 (d, 2H, J=8.4 Hz), 4.54 (s, 2H), 3.79 (s, 2H), 2.68 (s, 6H), 2.43 (t, 2H, J=6.0 Hz), 2.11 (s, 3H), 2.04 (s, 2H), 1.54 (t, 2H, J=6.4 Hz), 0.91 (s, 6H).

### Scheme 3—Synthesis of 4-(pyrrolidin-1-ylsulfonyl)benzaldehyde (23)

### Scheme 3a: Preparation of (4-(Chlorosulfonyl)phenyl)methylene diacetate (21)

To a solution of p-toluenesulfonyl chloride (20) (4.02 g, 21.1 mmol) in Ac₂O (40 mL, 424 mmol) and AcOH (40 mL) at 0° C. was added sulfuric acid (6.4 mL). Solid chromium trioxide (8.61 g, 84.4 mmol) was added over 5 min, and the resulting solution was stirred for 30 min at 0° C., leading to the formation of a white precipitate. The reaction was then filtered, and the resulting white solid was washed with water (35 mL), slurried in saturated sodium bicarbonate and stirred for 2 h. The solution was extracted with ethyl acetate (3×50 mL), dried (sodium sulfate), filtered, and concentrated to a white solid (4-(chlorosulfonyl)phenyl)methylene diacetate (21) (2.5 g, 8.15 mmol, 38.7%) upon concentration. ¹H NMR (400 MHz, CDCl₃) δ (ppm): 8.09 (d, 2H, J=8.8 Hz), 7.78 (d, 2H, J=8.4 Hz), 7.74 (s, 1H), 2.17 (s, 6H).

### Scheme 3b: Preparation of (4-(Pyrrolidin-1-ylsulfonyl)phenyl)methylene diacetate (22)

To a solution of (4-(chlorosulfonyl)phenyl)methylene diacetate (21) (2.54 g, 8.28 mmol) in acetone (24 mL) was added a solution of pyrrolidine (1.37 mL, 16.6 mmol) in acetone (3 mL) at room temperature. The reaction was stirred at room temperature overnight (14 h), then concentrated, redissolved in dichloromethane (50 mL), washed with 1N HCl (2×50 mL), and concentrated to a dark oil, (4-(pyrrolidin-1-ylsulfonyl)phenyl)methylene diacetate (22) (2.81 g, 8.23 mmol, 99%). ¹H NMR (400 MHz, CDCl₃) δ (ppm): 7.87 (d, 2H, J=8.4 Hz), 7.72 (s, 1H), 7.67 (d, 2H, J=8.4 Hz), 3.26 (t, 4H, J=6.8 Hz), 2.16 (s, 6H), 1.79 (m, 4H).

### Scheme 3c: Preparation of 4-(Pyrrolidin-1-ylsulfonyl)benzaldehyde (23)

To a solution of (4-(pyrrolidin-1-ylsulfonyl)phenyl)methylene diacetate (22) (2.82 g, 8.26 mmol) in ethanol (10 mL) and water (10 mL) was added sulfuric acid (1.00 mL, 18.8 mmol). The reaction was refluxed for 2.5 h, after which it was cooled to room temperature. Removal of ethanol in vacuo, followed by cooling to 0° C. led to a precipitate which was collected and washed with water. A tan crystalline solid was collected as 4-(pyrrolidin-1-ylsulfonyl)benzaldehyde (23) (1.81 g, 7.56 mmol, 92%). ¹H NMR (400 MHz, CDCl₃) δ (ppm): 10.1 (s, 1H), 8.05 (d, 2H, J=8.4 Hz), 8.01 (d, 2H, J=8.4 Hz), 3.28 (t, 4H, J=6.4 Hz), 1.80 (m, 4H).

### Scheme 4—Synthesis of 2-(pyrrolidin-1-ylsulfonyl)benzaldehyde (27)

### Scheme 4a: Preparation of 1-(Phenylsulfonyl)pyrrolidine (25)

To a solution of benzenesulfonyl chloride (24) (7.30 mL, 56.6 mmol) in acetone (100 mL) was added neat pyrrolidine (9.36 mL, 113 mmol). The reaction was stirred at room temperature for 4 h, after which it was concentrated, extracted w/dichloromethane (3×75 mL), dried (sodium sulfate), filtered and concentrated to an off-white crystalline solid 1-(phenylsulfonyl)pyrrolidine (25) (10.3 g, 48.8 mmol, 86%). ¹H NMR (400 MHz, CDCl₃) δ (ppm): 7.83 (m, 2H), 7.50-7.61 (m, 3H), 3.25 (m, 4H), 1.75 (m, 4H).

### Scheme 4b: Preparation of Sodium hydroxy(2-(pyrrolidin-1-ylsulfonyl)phenyl)methanesulfonate (26)

To a solution of 1-(phenylsulfonyl)pyrrolidine (25) (4.88 g, 23.10 mmol) in THF (47.0 mL) at 0° C. was added a hexane solution of n-BuLi (13.93 mL, 27.7 mmol). The reaction was stirred at 0° C. for 30 min, after which a solution of morpholine-4-carbaldehyde (2.80 mL, 27.7 mmol) in THF (28 mL) was added. The reaction was stirred at 0° C. for 1 h, after which an aliquot was removed and quenched with water, then concentrated to dryness. The reaction was allowed to warm to room temperature and stirred for 14 h (overnight), then quenched by addition of water (40 mL) and the organic layer was separated. The aqueous layer was treated with solid NaCl, and then extraction was performed with diethyl ether (3×75 mL). The combined organic extracts were washed with saturated sodium chloride solution (2×100 mL), dried (sodium sulfate), filtered and concentrated to a dark brown residue, which was shown by ¹H NMR to be a 2:1 mixture of 2-(pyrrolidin-1-ylsulfonyl)benzaldehyde (4.9 g, 13.72 mmol, 59.4%) to 1-(phenylsulfonyl)pyrrolidine. The solid mixture was dissolved in Et₂O (20 mL) to which a solution of sodium bisulfite (2.86 g, 27.4 mmol) in water (20 mL) was added. The solution was stirred at room temperature for 2 h, leading to the formation of an off-white precipitate which was washed in ether then hexanes. The first crop afforded 3.33 g of analytically pure bisulfite adduct and the second crop (refiltered material) afforded 293 mg. The off white solid, sodium hydroxy(2-(pyrrolidin-1-ylsulfonyl)phenyl)methanesulfonate (26), (3.623 g, 10.55 mmol, 77%) was dried in vacuo. ¹H NMR (400 MHz, D₂O) δ (ppm): 7.92 (d, 1H, J=8.0 Hz), 7.83 (d, 1H, J=8.0 Hz), 7.61 (t, 1H, J=7.8 Hz), 7.49 (t, 1H, J=7.8 Hz), 6.33 (s, 1H), 3.08-3.15 (m, 4H), 1.70 (m, 4H).

### Scheme 4c: Preparation of 2-(Pyrrolidin-1-ylsulfonyl)benzaldehyde (27)

To a solution of sodium hydroxy(2-(pyrrolidin-1-ylsulfonyl)phenyl)methanesulfonate (26) (2.79 g, 8.13 mmol) in THF (16 mL) and water (16 mL) was added sodium carbonate (1.72 g, 16.3 mmol). The reaction was heated to 75° C. for 1.5 h, then cooled to room temperature, concentrated to ˜½ volume by removing the THF in vacuo, extracted with dichloromethane (3×50 mL), dried (sodium sulfate), filtered, and concentrated to a gold oil which solidified on standing affording 2-(pyrrolidin-1-ylsulfonyl)benzaldehyde (27) (1.65 g, 6.90 mmol, 85%). ¹H NMR (400 MHz, CDCl₃) δ (ppm): 10.9 (s, 1H), 8.07 (m, 1H), 7.98 (m, 1H), 7.72 (m, 2H), 3.29 (t, 4H, J=6.4 Hz), 1.87 (m, 4H).

### Scheme 5—Synthesis of 2-(2-methyl-3-(4-(pyrrolidin-1-ylsulfonyl)benzoyl)-4,5,6,7-tetrahydro-1H-indol-1-yl)acetic acid (33)

### Scheme 5a: Preparation of 2-(2-Oxopropyl)cyclohexanone (29)

NaH (0.45 g 60% in mineral oil, 11.21 mmol) was added to DMF (16.00 mL) at 25° C. cyclohexanone (28) (1.0 g, 10.19 mmol) was added, stirred for 10 min, then heated at 40° C. for 3 h, 3-chloro-2-(methoxymethoxy)prop-1-ene (1.39 mL, 11.21 mmol) was added dropwise and heated at 120° C. for 1 h, cooled to room temperature, quenched with MeOH (1 mL) and the solvent was washed with water, extracted with dichloromethane, combined extracts were washed with brine, dried with Na₂SO₄, filtered and concentrated, to this residue was added 1% H₂SO₄ (5 mL) and dioxane (5 mL), heated at 60° C. for 1 h, the product was extracted with ether, combined extracts was dried with Na₂SO₄, filtered and concentrated, the residue was purified by Biotage, to give 2-(2-oxopropyl)cyclohexanone (29) as a liquid (0.36 g, 2.33 mmol, 23%). ¹H NMR (400 MHz, CDCl₃) δ (ppm): 2.90-2.99 (m, 2H), 2.31-2.41 (m, 2H), 2.19 (s, 3H), 2.03-2.17 (m, 3H), 1.55-1.89 (m, 3H), 1.30-1.41 (m, 1H).

### Scheme 5b: Preparation of Ethyl 2-(2-methyl-4,5,6,7-tetrahydro-1H-indol-1-yl)acetate (30)

2-(2-Oxopropyl)cyclohexanone (29) (1.78 g, 11.54 mmol) in dichloromethane (5 mL) was stirred, ethyl 2-aminoacetate hydrochloride (1.93 g, 13.85 mmol) was added, followed by sodium bicarbonate (1.46 g, 17.31 mmol), the mixture was stirred at room temperature for 48 h, dichloromethane was added, washed with water, brine, dried with Na₂SO₄, filtered and concentrated, the residue was chromatographed via by Biotage, and eluted with ethyl acetate in hexanes (5 to 50%), to give ethyl 2-(2-methyl-4,5,6,7-tetrahydro-1H-indol-1-yl)acetate (30) 1.8 g as a liquid (8.13 mmol, 70.5%). ¹H NMR (400 MHz, CDCl₃) δ (ppm): 5.72 (s, 1H), 4.43 (s, 2H), 4.23 (q, 2H, J=6.8 Hz), 2.45 (m, 4H), 2.17 (s, 3H), 1.83 (m, 2H), 1.73 (m, 2H), 1.29 (t, 3H, J=7.0 Hz).

### Scheme 5c: Preparation of 4-(Pyrrolidin-1-ylsulfonyl)benzoyl chloride(31)

4-(Pyrrolidin-1-ylsulfonyl)benzoic acid (2.10 g, 8.23 mmol) in 250 mL of flask was stirred and SOCl₂ (4.20 mL, 57.6 mmol) was added, the mix was stirred and additional SOCl₂ (4.20 mL, 57.6 mmol) was added, the mixture was heated to reflux, after the solid was dissolved and the solution was clear, the thionylchloride was removed to give 4-(pyrrolidin-1-ylsulfonyl)benzoyl chloride (31) as a solid (1.98 g, 7.23 mmol, 88%). ¹H NMR (400 MHz, CDCl₃) δ (ppm): 8.19 (d, 2H, J=8.4 Hz), 7.89 (d, 2H, J=8.4 Hz), 3.32 (t, 4H, J=6.8 Hz), 1.84 (m, 4H).

### Example 15

### Ethyl 2-(2-methyl-3-(4-(pyrrolidin-1-ylsulfonyl)benzoyl)-4,5,6,7-tetrahydro-1H-indol-1-yl)acetate (I-99)

To a stirred solution of ethyl 2-(2-methyl-4,5,6,7-tetrahydro-1H-indol-1-yl)acetate (154 mg, 0.70 mmol) (31) in dichloromethane (5 mL) was added diethylaluminum chloride (0.77 mL, 0.77 mmol) at 0° C., stirred for 30 min then 4-(pyrrolidin-1-ylsulfonyl)benzoyl chloride (210 mg, 0.77 mmol) in dichloromethane (1 mL) was added, stirred at 0° C. for 3 h then slowly warmed to room temperature overnight. The reaction was poured into ice cooled 1N HCl and extracted with dichloromethane, combined extracts were washed with water and brine, dried with Na₂SO₄, filtered and concentrated, the residue was chromatographed via Biotage, (eluted with ethyl acetate in hexanes (with 1% TEA) (5 to 75%) to give ethyl 2-(2-methyl-3-(4-(pyrrolidin-1-ylsulfonyl)benzoyl)-4,5,6,7-tetrahydro-1H-indol-1-yl)acetate (32) as a yellow a solid (215 mg, 0.47 mmol, 65.8%). ¹H NMR (400 MHz, CDCl₃) δ (ppm): 7.87 (d, 2H, J=8.4 Hz), 7.78 (d, 2H, J=8.4 Hz), 4.50 (s, 2H), 4.25 (q, 2H, J=7.2 Hz), 3.25 (m, 4H), 2.43 (m, 2H), 2.23 (s, 3H), 2.14 (t, 2H, J=6.4 Hz), 1.74-1.79 (m, 6H), 1.58 (m, 2H), 1.31 (t, 3H, J=6.8 Hz).

### Example 16

### 2-(2-Methyl-3-(4-(pyrrolidin-1-ylsulfonyl)benzoyl)-4,5,6,7-tetrahydro-1H-indol-1-yl)acetic acid (I-100)

Ethyl 2-(2-methyl-3-(4-(pyrrolidin-1-ylsulfonyl)benzoyl)-4,5,6,7-tetrahydro-1H-indol-1-yl)acetate (32) (50 mg, 0.11 mmol) in THF (2.50 mL), MeOH (0.83 mL) and water (0.83 mL) was stirred, LiOH (5.22 mg, 0.22 mmol) was added, stirred at 0° C. for 2 h, TLC revealed that the starting material was gone, remove solvent, water was added, followed by 1 N HCl, remove solvent, MeOH was added, purified by prep-HPLC to give 37 mg of 2-(2-methyl-3-(4-(pyrrolidin-1-ylsulfonyl)benzoyl)-4,5,6,7-tetrahydro-1H-indol-1-yl)acetic acid (33) as a light blue solid, (0.09 mmol, 79%). ¹H NMR (400 MHz, DMSO d₆) δ (ppm): 7.86 (d, 2H, J=8.4 Hz), 7.69 (d, 2H, J=8.0 Hz), 4.63 (s, 2H), 3.15 (t, 4H, J=6.8 Hz), 2.38 (t, 2H, J=6.0 Hz), 2.09 (s, 3H), 2.02 (t, 2H, J=5.6 Hz), 1.59-1.68 (m, 6H), 1.48 (m, 2H).

### Scheme 6—General Synthesis for Benzoyl Dimethyl Tetrahydroindols (36)

### Example 17

### Ethyl 2-(2,5,5-trimethyl-3-(4-(pyrrolidin-1-ylsulfonyl)benzoyl)-4,5,6,7-tetrahydro-1H-indol-1-yl)acetate (I-101)

To a stirred solution of ethyl 2-(2,5,5-trimethyl-4,5,6,7-tetrahydro-1H-indol-1-yl)acetate (3) (100 mg, 0.40 mmol) in dichloromethane (5 mL) was added diethylaluminum chloride (0.44 mL, 0.44 mmol) at 0° C., stirred for 30 min then 4-(pyrrolidin-1-ylsulfonyl)benzoyl chloride (121 mg, 0.44 mmol) in dichloromethane (1 mL) was added, stirred at 0° C. for 3 h then slowly warmed to room temperature overnight, poured into ice cooled 1N HCl and extracted with dichloromethane, combined extracts were washed with water and brine, dried with Na₂SO₄, filtered and concentrated. The residue was chromatographed via Biotage, and eluted with ethyl acetate in hexanes (with 1% TEA) (5 to 75%) to give ethyl 2-(2,5,5-trimethyl-3-(4-(pyrrolidin-1-ylsulfonyl)benzoyl)-4,5,6,7-tetrahydro-1H-indol-1-yl)acetate (37) as a yellow solid (95.8 mg, 0.20 mmol, 49.1%). ¹H NMR (400 MHz, CDCl₃) δ (ppm): 7.85 (d, 2H, J=8.4 Hz), 7.77 (d, 2H, J=8.4 Hz), 4.49 (s, 2H), 4.22 (q, 2H, J=7.2 Hz), 3.23 (m, 4H), 2.40 (m, 2H), 2.20 (s, 3H), 1.93 (s, 2H), 1.72 (m, 4H), 1.52 (t, 2H, J=6.4 Hz), 1.27 (t, 3H, J=7.2 Hz), 0.82 (s, 6H).

### Example 18

### 2-(2,5,5-Trimethyl-3-(4-(pyrrolidin-1-ylsulfonyl)benzoyl)-4,5,6,7-tetrahydro-1H-indol-1-yl)acetic acid (I-102)

Ethyl 2-(2,5,5-trimethyl-3-(4-(pyrrolidin-1-ylsulfonyl)benzoyl)-4,5,6,7-tetrahydro-1H-indol-1-yl)acetate (95 mg, 0.20 mmol) in THF (2.0 mL), MeOH (0.50 mL) and water (0.50 mL) was stirred, LiOH (18.70 mg, 0.78 mmol) was added, stirred at 25° C. for 2 h, TLC showed that starting material was gone, remove solvent, water was added, followed by 1 N HCl, remove solvent, MeOH was added, purified by prep-HPLC to give 2-(2,5,5-trimethyl-3-(4-(pyrrolidin-1-ylsulfonyl)benzoyl)-4,5,6,7-tetrahydro-1H-indol-1-yl)acetic acid (38) as a yellow solid, (66 mg, 0.14 mmol 73.7%). ¹H NMR (400 MHz, CDCl₃) δ (ppm): 7.87 (d, 2H, J=8.4 Hz), 7.78 (d, 2H, J=8.4 Hz), 4.57 (s, 2H), 3.25 (t, 4H, J=6.8 Hz), 2.43 (t, 2H, J=6.0 Hz), 2.23 (s, 3H), 1.93 (s, 2H), 1.74 (m, 4H), 1.54 (t, 2H, J=6.4 Hz), 0.83 (s, 6H).

The following compounds are made in same fashion as 2-(2,5,5-trimethyl-3-(4-(pyrrolidin-1-ylsulfonyl)benzoyl)-4,5,6,7-tetrahydro-1H-indol-1-yl)acetic acid (I-4).

### Example 19

### 2-(2,5,5-Trimethyl-3-(4-(morpholinosulfonyl)benzoyl)-4,5,6,7-tetrahydro-1H-indol-1-yl)acetic acid (I-103)

¹H NMR (400 MHz, CDCl₃) δ (ppm): 7.82 (d, 2H, J=8.8 Hz), 7.80 (d, 2H, J=8.6 Hz), 4.59 (s, 2H), 3.75 (m, 4H), 3.01 (t, 4H, J=4.4 Hz), 2.44 (t, 2H, J=6.4 Hz), 2.25 (s, 3H), 1.94 (s, 2H), 1.56 (t, 2H, J=6.4 Hz), 0.85 (s, 6H).

### Example 20

### 2-(2,5,5-Trimethyl-3-(4-(4-methylpiperidin-1-ylsulfonyl)benzoyl)-4,5,6,7-tetrahydro-1H-indol-1-yl)acetic acid (I-104)

¹H NMR (400 MHz, CDCl₃) δ (ppm): 7.80 (d, 2H, J=9.2 Hz), 7.78 (d, 2H, J=8.4 Hz), 4.59 (s, 2H), 3.76 (d, 2H, J=11.2 Hz), 2.44 (t, 2H, J=6.0 Hz), 2.22 (m, 5H), 1.95 (s, 2H), 1.67 (d, 2H, J=10.0 Hz), 1.55 (t, 2H, J=6.4 Hz), 1.29 (m, 3H), 0.91 (d, 3H, J=4.8 Hz), 0.83 (s, 6H).

### Example 21

### 2-(3-(4-(3,5-Dimethylpiperidin-1-ylsulfonyl)benzoyl)-2,5,5-trimethyl-4,5,6,7-tetrahydro-1H-indol-1-yl)acetic acid (I-105)

¹H NMR (400 MHz, CDCl₃, cis:trans=9:1, δ (ppm): 7.80 (d, 2H, J=8.8 Hz), 7.78 (d, 2H, J=8.4 Hz), 4.58 (s, 2H), 3.73 (d, 2H, J=9.2 Hz), 2.43 (t, 2H, J=6.0 Hz), 2.25 (s, 3H), 1.92 (s, 2H), 1.63-1.77 (m, 4H), 1.55 (t, 2H, J=6.4 Hz), 0.97 (d, 1H, J=6.8 Hz), 0.84 (m, 12H), 0.44 (q, 1H, J=12 Hz).

### Scheme 7: Preparation of Ethyl 2-(2-methyl-4,5,6,7-tetrahydro-1H-indol-1-yl)acetate (30)

Scheme 7A: Preparation of 2-(2-Oxopropyl)cyclohexanone (29). NaH (0.45 g 60% in mineral oil, 11.21 mmol) was added to DMF (16.00 mL) at 25° C. cyclohexanone (28) (1.0 g, 10.19 mmol) was added, stirred for 10 min, then heated at 40° C. for 3 h, 3-chloro-2-(methoxymethoxy)prop-1-ene (1.39 mL, 11.21 mmol) was added dropwise and heated at 120° C. for 1 h, cooled to room temperature, quenched with MeOH (1 mL) and the solvent was washed with water, extracted with dichloromethane, combined extracts were washed with brine, dried with Na₂SO₄, filtered and concentrated, to this residue was added 1% H₂SO₄ (5 mL) and dioxane (5 mL), heated at 60° C. for 1 h, the product was extracted with ether, combined extracts was dried with Na₂SO₄, filtered and concentrated, the residue was purified by Biotage, to give 2-(2-oxopropyl)cyclohexanone (29) as a liquid (0.36 g, 2.33 mmol, 23%). ¹H NMR (400 MHz, CDCl₃) δ (ppm): 2.90-2.99 (m, 2H), 2.31-2.41 (m, 2H), 2.19 (s, 3H), 2.03-2.17 (m, 3H), 1.55-1.89 (m, 3H), 1.30-1.41 (m, 1H).

Scheme 7B: Preparation of Ethyl 2-(2-methyl-4,5,6,7-tetrahydro-1H-indol-1-yl)acetate (30). 2-(2-Oxopropyl)cyclohexanone (29) (1.78 g, 11.54 mmol) in dichloromethane (5 mL) was stirred, ethyl 2-aminoacetate hydrochloride (1.93 g, 13.85 mmol) was added, followed by sodium bicarbonate (1.46 g, 17.31 mmol), the mixture was stirred at room temperature for 48 h, dichloromethane was added, washed with water, brine, dried with Na₂SO₄, filtered and concentrated, the residue was chromatographed via by Biotage, and eluted with ethyl acetate in hexanes (5 to 50%), to give ethyl 2-(2-methyl-4,5,6,7-tetrahydro-1H-indol-1-yl)acetate (30) 1.8 g as a liquid (8.13 mmol, 70.5%). ¹H NMR (400 MHz, CDCl₃) δ (ppm): 5.72 (s, 1H), 4.43 (s, 2H), 4.23 (q, 2H, J=6.8 Hz), 2.45 (m, 4H), 2.17 (s, 3H), 1.83 (m, 2H), 1.73 (m, 2H), 1.29 (t, 3H, J=7.0 Hz)

### Scheme 8: General Synthesis for Functionalized Tetrahydroindoles (44)

Scheme 8A: Preparation of 1-(4-(Bromomethyl)phenylsulfonyl)pyrrolidine (Generic compound 42, wherein R is pyrrolidine). Pyrrolidine (1.53 mL, 18.55 mmol) in dichloromethane (20 mL) was stirred at 0° C., triethylamine (2.84 mL, 20.40 mmol) was added, followed by 4-(bromomethyl)benzene-1-sulfonyl chloride (45) (5.0 g, 18.55 mmol) slowly warmed to room temperature overnight, TLC showed that starting material was gone, dichloromethane was added, washed with water, brine, dried with Na₂SO₄, filtered and concentrated, the residue was chromatographed via Biotage, and eluted with (10 to 50%) Ethyl acetate in hexanes to give 1-(4-(bromomethyl)phenylsulfonyl)pyrrolidine (42) as a solid (3.07 g, 10.09 mmol, 54.4%). ¹H NMR (400 MHz, CDCl₃) δ (ppm): 7.78 (d, 2H, J=8.0 Hz), 7.52 (d, 2H, J=8.0 Hz), 4.60 (s, 2H), 3.21 (m, 4H), 1.73 (m, 4H).

### Example 21

Preparation of Ethyl 2-(2-methyl-3-(4-(pyrrolidin-1-ylsulfonyl)benzyl)-4,5,6,7-tetrahydro-1H-indol-1-yl)acetate (Generic compound 43, wherein R is pyrrolidine, I-31) Ethyl 2-(2-methyl-4,5,6,7-tetrahydro-1H-indol-1-yl)acetate (335 mg, 1.51 mmol) (30) and 1-(4-(bromomethyl)phenylsulfonyl)pyrrolidine (460 mg, 1.51 mmol) in acetonitrile (2 mL) was stirred, potassium carbonate (418 mg, 3.02 mmol) was added, the mixture was stirred at 80° C. for 24 h, cooled to room temperature and purified via Biotage, and eluted with ethyl acetate in hexanes (5 to 30%) to give Ethyl 2-(2-methyl-3-(4-(pyrrolidin-1-ylsulfonyl)benzyl)-4,5,6,7-tetrahydro-1H-indol-1-yl)acetate (I-31), as a solid (120 mg, 0.27 mmol, 17.9%). ¹H NMR (400 MHz, CDCl₃) δ (ppm): 7.69 (d, 2H, J=8.0 Hz), 7.29 (d, 2H, J=8.0 Hz), 4.46 (s, 2H), 4.22 (q, 2H, J=6.8 Hz), 3.78 (s, 2H), 3.22 (m, 4H), 2.43 (m, 2H), 2.23 (m, 2H), 2.11 (s, 3H), 1.64-1.79 (m, 8H), 1.29 (t, 3H, J=7.0 Hz).

### Example 22

Preparation of 2-(2-Methyl-3-(4-(pyrrolidin-1-ylsulfonyl)benzyl)-4,5,6,7-tetrahydro-1H-indol-1-yl)acetic acid (Generic compound 44, wherein R is pyrrolidine, I-1) Ethyl 2-(2-methyl-3-(4-(pyrrolidin-1-ylsulfonyl)benzyl)-4,5,6,7-tetrahydro-1H-indol-1-yl)acetate (I-31) (140 mg, 0.32 mmol) in THF (2.50 mL), MeOH (0.83 mL) and Water (0.83 mL) was stirred, LiOH (15.08 mg, 0.63 mmol) was added, stirred at 0° C. for 2 h, TLC showed that starting material was gone, remove solvent, water was added, followed by 1 N HCl, remove solvent, MeOH was added, purified by prep-HPLC to give 2-(2-methyl-3-(4-(pyrrolidin-1-ylsulfonyl)benzyl)-4,5,6,7-tetrahydro-1H-indol-1-yl)acetic acid (I-1) as a light blue solid (70 mg, 0.17 mmol, 53.4%). ¹H NMR (400 MHz, CDCl₃) δ (ppm): 7.69 (d, 2H, J=8.4 Hz), 7.28 (d, 2H, J=8.8 Hz), 4.54 (s, 2H), 3.78 (s, 2H), 3.22 (m, 4H), 2.44 (t, 2H, J=4.0 Hz), 2.24 (t, 2H, J=4.4 Hz), 2.12 (s, 3H), 1.63-1.81 (m, 8H).

The following compounds are made by the same process as 2-(2-methyl-3-(4-(pyrrolidin-1-ylsulfonyl)benzyl)-4,5,6,7-tetrahydro-1H-indol-1-yl)acetic acid (I-1) according to Scheme 8.

### Example 23

2-(3-(4-(N-Cyclohexylsulfamoyl)benzyl)-2-methyl-4,5,6,7-tetrahydro-1H-indol-1-yl)acetic acid (I-2). ¹H NMR (400 MHz, CDCl₃) δ (ppm): 7.74 (d, 2H, J=8.4 Hz), 7.28 (d, 2H, J=8.4 Hz), 4.68 (m, 1H), 4.54 (s, 2H), 3.78 (s, 2H), 3.11 (m, 1H), 2.44 (t, 2H, J=6.0 Hz), 2.21 (t, 2H, J=6.0 Hz), 2.12 (s, 3H), 1.60-1.80 (m, 8H), 1.50 (m, 1H), 1.09-1.23 (m, 5H).

### Example 24

2-(2-Methyl-3-(4-(morpholinosulfonyl)benzyl)-4,5,6,7-tetrahydro-1H-indol-1-yl)acetic acid (I-6. ¹H NMR (400 MHz, CDCl₃) δ (ppm): 7.61 (d, 2H, J=8.4 Hz), 7.31 (d, 2H, J=8.4 Hz), 4.54 (s, 2H), 3.80 (s, 2H), 3.74 (m, 4H), 2.98 (m, 4H), 2.45 (t, 2H, J=6.0 Hz), 2.25 (t, 2H, J=6.0 Hz), 2.12 (s, 3H), 1.80 (m, 2H), 1.69 (m, 2H).

### Example 25

2-(3-(4-(4-(Ethoxycarbonyl)piperazin-1-ylsulfonyl)benzyl)-2-methyl-4,5,6,7-tetrahydro-1H-indol-1-yl)acetic acid (I-5). ¹H NMR (400 MHz, CDCl₃) δ (ppm): 7.60 (d, 2H, J=8.8 Hz), 7.30 (d, 2H, J=8.4 Hz), 4.52 (s, 2H), 4.08 (q, 2H, J=7.2 Hz), 3.79 (s, 2H), 3.55 (m, 4H), 2.96 (m, 4H), 2.44 (t, 2H, J=6.0 Hz), 2.25 (t, 2H, J=5.6 Hz), 2.11 (s, 3H), 1.78 (m, 2H), 1.69 (m, 2H), 1.21 (t, 3H, J=6.8 Hz).

### Example 26

2-(3-(4-(N-Cyclopropylsulfamoyl)benzyl)-2-methyl-4,5,6,7-tetrahydro-1H-indol-1-yl)acetic acid (I-8). ¹H NMR (400 MHz, CDCl₃) δ (ppm): 7.77 (d, 2H, J=8.4 Hz), 7.29 (d, 2H, J=8.4 Hz), 5.16 (m, 1H), 4.54 (s, 2H), 3.80 (s, 2H), 2.45 (t, 2H, J=6.0 Hz), 2.25 (m, 3H), 2.12 (s, 3H), 1.80 (m, 2H), 1.68 (m, 2H), 0.56-0.61 (m, 4H).

### Example 27

2-(3-(4-(N-Isopropylsulfamoyl)benzyl)-2-methyl-4,5,6,7-tetrahydro-1H-indol-1-yl)acetic acid (I-10). ¹H NMR (400 MHz, CDCl₃) δ (ppm): 7.74 (d, 2H, J=8.4 Hz), 7.26 (d, 2H, J=8.0 Hz), 4.84 (d, 1H, J=7.6 Hz), 4.53 (s, 2H), 3.79 (s, 2H), 3.42 (m, 1H), 2.45 (t, 2H, J=6.0 Hz), 2.22 (t, 2H, J=6.0 Hz), 2.12 (s, 3H), 1.79 (m, 2H), 1.67 (m, 2H), 1.06 (d, 6H, J=6.4 Hz).

### Example 28

2-(3-(4-(N-(3,3-Dimethylbutan-2-yl)sulfamoyl)benzyl)-2-methyl-4,5,6,7-tetrahydro-1H-indol-1-yl)acetic acid (I-3). ¹H NMR (400 MHz, CDCl₃) δ (ppm): 7.74 (d, 2H, J=8.4 Hz), 7.25 (d, 2H, J=8.0 Hz), 4.74 (d, 1H, J=9.6 Hz), 4.53 (s, 2H), 3.78 (s, 2H), 3.02 (m, 1H), 2.44 (t, 2H, J=6.0 Hz), 2.18 (t, 2H, J=6.0 Hz), 2.11 (s, 3H), 1.78 (m, 2H), 1.65 (m, 2H), 0.86 (d, 3H, J=6.8 Hz), 0.82 (s, 9H).

The compound I-7 was synthesized via general route for compound I-14 using ethyl 2-(2-methyl-4,5,6,7-tetrahydro-1H-indol-1-yl)acetate (30) as the starting material.

### Example 29

2-(2-methyl-3-(2-(phenylsulfonyl)benzyl)-4,5,6,7-tetrahydro-1H-indol-1-yl)acetic acid (I-7). ¹H NMR (400 MHz, CDCl₃) δ (ppm): 8.29 (dd, 1H, J=7.6 Hz, J=1.2 Hz), 7.89 (m, 2H), 7.57 (m, 1H), 7.38-7.51 (m, 4H), 7.00 (dd, 1H, J=8.4 Hz, J=0.8 Hz), 4.48 (s, 2H), 3.85 (s, 2H), 2.40 (t, 2H, J=6.0 Hz), 1.82 (s, 3H), 1.72 (m, 4H), 1.52 (m, 2H).

### Scheme 9: Synthesis of ethyl 2-(2-methyl-3-(4-(pyrrolidin-1-ylsulfonyl)benzyl)-5,6-dihydrocyclopenta[b]pyrrol-1(4H)-yl)acetate (I-32) and 2-(2-methyl-3-(4-(pyrrolidin-1-ylsulfonyl)benzyl)-5,6-dihydrocyclopenta[b]pyrrol-1(4H)-yl)acetic acid (I-19)

Scheme 9A: Preparation of 2-(2-Oxopropyl)cyclopentanone (56). Ammonium cerium(IV) nitrate (7.02 g, 12.80 mmol) and sodium bicarbonate (2.150 g, 25.6 mmol) in acetonitrile (25 mL) was stirred at 25° C., cyclopentenyloxytrimethylsilane (1.14 mL, 6.40 mmol) and trimethyl(prop-1-en-2-yloxy)silane (9.69 mL, 64.0 mmol) were added dropwise, stirred until the orange color disappeared and a thick white precipitate formed. The reaction mix was poured into water and extracted with ethyl acetate, combined extracts were washed with brine, and dried with Na₂SO₄, filtered and concentrated, the residue was purified by chromatography over silica gel, eluted with ethyl acetate in hexanes (10 to 50%) to give 2-(2-oxopropyl)cyclopentanone (56) (0.44 g, 3.14 mmol, 49.1%). ¹H NMR (400 MHz, CDCl₃) δ (ppm): 2.91 (dd, 1H, J=18.0 Hz, J=3.6 Hz), 2.69 (s, 1H), 2.40-2.55 (m, 2H), 2.18-2.37 (m, 2H), 2.15 (s, 3H), 1.99-2.07 (m, 1H), 1.73-1.85 (m, 1H), 1.46-1.56 (m, 1H).

Scheme 9B: Preparation of Ethyl 2-(2-methyl-5,6-dihydrocyclopenta[b]pyrrol-1(4H)-yl)acetate (57). Sodium sulfate (0.45 g, 3.14 mmol) in dichloromethane (15 mL) was stirred, ethyl 2-aminoacetate (0.39 g, 3.77 mmol) and 2-(2-oxopropyl)cyclopentanone (56) (0.44 g, 3.14 mmol) were added, followed by TEA (0.44 mL, 3.14 mmol), the mixture was stirred at room temperature for 24 h, dichloromethane was added, washed with water, brine, dried with Na₂SO₄ filtered and concentrated, the residue was chromatographed via Biotage, and eluted with ethyl acetate in hexane (5 to 50%), to give ethyl 2-(2-methyl-5,6-dihydrocyclopenta[b]pyrrol-1(4H)-yl)acetate (57) 171 mg, 0.83 mmol, 26.3%. ¹H NMR (400 MHz, CDCl₃) δ (ppm): 5.72 (s, 1H), 4.44 (s, 2H), 4.24 (q, 2H, J=6.8 Hz), 2.60 (m, 4H), 2.38 (m, 2H), 2.17 (s, 3H), 1.28 (t, 3H, J=7.2 Hz).

### Example 30

Preparation of Ethyl 2-(2-methyl-3-(4-(pyrrolidin-1-ylsulfonyl)benzyl)-5,6-dihydrocyclopenta[b]pyrrol-1(4H)-yl)acetate (I-32) To a flame-dried vial was charged with dichloromethane (2 mL), trimethylsilyl trifluoromethanesulfonate (0.087 mL, 0.482 mmol was added at 0° C., followed by a solution of ethyl 2-(2-methyl-5,6-dihydrocyclopenta[b]pyrrol-1(4H)-yl)acetate (57) (50 mg, 0.24 mmol and 4-(pyrrolidin-1-ylsulfonyl)benzaldehyde (57.7 mg, 0.24 mmol in dichloromethane (5 mL) (cannulated), the mix was stirred at 0° C. for 15 min, neat triethylsilane (0.15 mL, 0.97 mmol) was added slowly, stirred for 30 min at 0° C., then slowly warmed to room temperature. TLC checked that reaction was done, quenched with sat. NaHCO₃, extracted with dichloromethane. Combined extracts were dried with Na₂SO₄, filtered and concentrated, the residue was chromatographed via prep-HPLC to give ethyl 2-(2-methyl-3-(4-(pyrrolidin-1-ylsulfonyl)benzyl)-5,6-dihydrocyclopenta[b]pyrrol-1(4H)-yl)acetate (I-32) as a solid (37 mg, 0.086 mmol, 35.6%). ¹H NMR (400 MHz, CDCl₃) δ (ppm): 7.70 (d, 2H, J=8.4 Hz), 7.32 (d, 2H, J=8.4 Hz), 4.44 (s, 2H), 4.22 (q, 2H, J=7.2 Hz), 3.78 (s, 2H), 3.21 (m, 4H), 2.59 (m, 2H), 2.32 (m, 4H), 2.11 (s, 3H), 1.74 (m, 4H), 1.28 (t, 3H, J=7.2 Hz).

### Example 31

Preparation of 2-(2-Methyl-3-(4-(pyrrolidin-1-ylsulfonyl)benzyl)-5,6-dihydrocyclopenta[b]pyrrol-1(4H)-yl)acetic acid (I-19). Ethyl 2-(2-methyl-3-(4-(pyrrolidin-1-ylsulfonyl)benzyl)-5,6-dihydrocyclopenta[b]pyrrol-1(4H)-yl)acetate (I-32) (37 mg, 0.09 mmol) in THF (2.0 mL), MeOH (0.50 mL) and water (0.50 mL) was stirred, LiOH (8.23 mg, 0.34 mmol) was added, stirred at 25° C. for 2 h, TLC checked that starting material was gone, remove solvent, water was added, followed by 1 N HCl, remove solvent, MeOH was added, purified by prep-HPLC to give 21 mg of 2-(2-methyl-3-(4-(pyrrolidin-1-ylsulfonyl)benzyl)-5,6-dihydrocyclopenta[b]pyrrol-1(4H)-yl)acetic acid (I-19) as a light blue solid, (0.05 mmol, 60.7%). ¹H NMR (400 MHz, CDCl₃) δ (ppm): 7.71 (d, 2H, J=8.0 Hz), 7.31 (d, 2H, J=8.4 Hz), 4.52 (s, 2H), 3.78 (s, 2H), 3.22 (t, 4H, J=6.4 Hz), 2.60 (m, 2H), 2.33 (m, 4H), 2.13 (s, 3H), 1.74 (m, 4H).

### Scheme 10: Synthesis of ethyl 2-(2-methyl-3-(2-(morpholinosulfonyl)benzyl)-5,6,7,8-tetrahydrocyclohepta[b]pyrrol-1(4H)-yl)acetate (I-33) and 2-(2-methyl-3-(2-(morpholinosulfonyl)benzyl)-5,6,7,8-tetrahydrocyclohepta[b]pyrrol-1(4H)-yl)acetic acid (I-21)

Scheme 10A: Preparation of 2-(2-Oxopropyl)cycloheptanone (61). NaH (1.24 g, 49.0 mmol) was added to toluene (16.00 mL) at 25° C. cycloheptanone (60) (5.5 g, 49.0 mmol) was added, stirred for 10 min, then heated at 40° C. for 3 h, 3-chloro-2-(methoxymethoxy)prop-1-ene (6.09 mL, 49.0 mmol) was added dropwise and heated at 120° C. for 3 h, cooled to 25° C., quenched with MeOH (1 mL) and the solvent was washed with water, extracted with dichloromethane, combined extracts were washed with brine, dried with Na₂SO₄, filtered and concentrated, to this residue was added 5 mL 1% H₂SO₄ and 5 mL dioxane, heated at 60° C. for 1 h, the product was extracted with ether, combined extracts was dried with Na₂SO₄, filtered and concentrated, the residue was purified by Biotage, to give 2-(2-oxopropyl)cycloheptanone (61) as a liquid 3.1 g, 18.4 mmol, 41.3%. ¹H NMR (400 MHz, CDCl₃) δ (ppm): 3.10-3.17 (m, 1H), 3.00-3.07 (m, 1H), 2.61-2.68 (m, 1H), 2.30-2.45 (m, 2H), 2.14 (s, 3H), 1.65-1.90 (m, 5H), 1.48-1.59 (m, 1H), 1.20-1.36 (m, 2H).

Scheme 10B: Preparation of Ethyl 2-(2-methyl-5,6,7,8-tetrahydrocyclohepta[b]pyrrol-1(4H)-yl)acetate (62). 2-(2-oxopropyl)cycloheptanone (61) (2.1 g, 12.48 mmol) in dichloromethane (3 mL) was stirred, ethyl 2-aminoacetate hydrochloride (2.61 g, 18.72 mmol) was added, followed by sodium bicarbonate (2.10 g, 24.97 mmol), the mixture was stirred at 55° C. for 5 h, TLC showed that reaction was done, dichloromethane was added, washed with water, brine, dried with Na₂SO₄, filtered and concentrated, the residue was chromatographed via Biotage, and eluted with ethyl acetate in hexanes 5 to 30%, to give ethyl 2-(2-methyl-5,6,7,8-tetrahydrocyclohepta[b]pyrrol-1(4H)-yl)acetate (62) as a liquid (2.70 g, 11.47 mmol, 92%). ¹H NMR (400 MHz, CDCl₃) δ (ppm): 5.69 (s, 1H), 4.48 (s, 2H), 4.23 (q, 2H, J=6.4 Hz), 2.54 (m, 4H), 2.15 (s, 3H), 1.78 (m, 2H), 1.67 (m, 4H), 1.28 (t, 3H, J=7.2 Hz).

### Example 32

Preparation of Ethyl 2-(2-methyl-3-(2-(morpholinosulfonyl)benzyl)-5,6,7,8-tetrahydrocyclohepta[b]pyrrol-1(4H)-yl)acetate (I-33) To a flame-dried vial was charged with dichloromethane (5 mL), trimethylsilyl trifluoromethanesulfonate (0.18 mL, 1.02 mmol) was added at 0° C., followed by a solution of ethyl 2-(2-methyl-5,6,7,8-tetrahydrocyclohepta[b]pyrrol-1(4H)-yl)acetate (62) (120 mg, 0.51 mmol) and 2-(morpholinosulfonyl)benzaldehyde (312 mg, 1.22 mmol) in dichloromethane (5 mL) (cannulated), the mixture was stirred at 0° C. for 15 min, neat triethylsilane (0.33 mL, 2.04 mmol) was added slowly, stirred for 30 min at 0° C., then slowly warmed to room temperature. TLC checked that the reaction was done, quenched with sat. NaHCO₃, extracted with dichloromethane. Combined extracts were dried with Na₂SO₄, filtered and concentrated, the residue was chromatographed via prep-HPLC to give ethyl 2-(2-methyl-3-(2-(morpholinosulfonyl)benzyl)-5,6,7,8-tetrahydrocyclohepta[b]pyrrol-1(4H)-yl)acetate (I-33) as a solid (60 mg, 0.13 mmol, 25%). ¹H NMR (400 MHz, CDCl₃) δ (ppm): 7.96 (d, 1H, J=7.6 Hz), 7.42 (t, 1H, J=7.6 Hz), 7.28 (t, 1H, J=7.2 Hz), 7.08 (d, 1H, J=8.0 Hz), 4.55 (s, 2H), 4.21 (q, 2H, J=7.2 Hz), 4.15 (s, 2H), 3.73 (t, 4H, J=4.6 Hz), 3.24 (t, 4H, J=4.6 Hz), 2.59 (t, 2H, J=4.6 Hz), 2.27 (t, 2H, J=5.4 Hz), 2.03 (s, 3H), 1.68-1.76 (m, 4H), 1.54 (m, 2H), 1.29 (t, 3H, J=7.2 Hz).

### Example 33

Preparation of 2-(2-Methyl-3-(2-(morpholinosulfonyl)benzyl)-5,6,7,8-tetrahydrocyclohepta[b]pyrrol-1(4H)-yl)acetic acid (I-21). Ethyl 2-(2-methyl-3-(2-(morpholinosulfonyl)benzyl)-5,6,7,8-tetrahydrocyclohepta[b]pyrrol-1(4H)-yl)acetate (I-33) (60 mg, 0.13 mmol) in THF (2 mL), MeOH (0.53 mL) and water (0.53 mL) was stirred, LiOH (6.06 mg, 0.25 mmol) was added, stirred at 25° C. for 2 h, TLC showed that starting material was gone, remove solvent, water was added, followed by 1 N HCl, remove solvent, MeOH was added, purified by prep-HPLC to give 44 mg of 2-(2-methyl-3-(2-(morpholinosulfonyl)benzyl)-5,6,7,8-tetrahydrocyclohepta[b]pyrrol-1(4H)-yl)acetic acid (I-21) as a light blue solid, (0.098 mmol, 78%). ¹H NMR (400 MHz, CDCl₃) δ (ppm): 7.97 (d, 1H, J=8.0 Hz), 7.42 (t, 1H, J=7.6 Hz), 7.29 (t, 1H, J=7.6 Hz), 7.05 (d, 1H, J=7.6 Hz), 4.62 (s, 2H), 4.16 (s, 2H), 3.74 (m, 4H), 3.25 (m, 4H), 2.61 (m, 2H), 2.29 (m, 2H), 2.05 (s, 3H), 1.69-1.77 (m, br, 4H), 1.55 (m, 2H).

The following compounds are made by a similar process as 2-(2-methyl-3-(2-(morpholinosulfonyl)benzyl)-5,6,7,8-tetrahydrocyclohepta[b]pyrrol-1(4H)-yl)acetic acid (I-21).

### Example 34

2-(2-Methyl-3-(4-(pyrrolidin-1-ylsulfonyl)benzyl)-5,6,7,8-tetrahydrocyclohepta[b]pyrrol-1(4H)-yl)acetic acid (I-20). ¹H NMR (400 MHz, CDCl₃) δ (ppm): 7.69 (d, 2H, J=8.8 Hz), 7.25 (d, 2H, J=8.0 Hz), 4.60 (s, 2H), 3.82 (s, 2H), 3.22 (t, 4H, J=6.8 Hz), 2.58 (t, 2H, J=4.8 Hz), 2.36 (t, 2H, J=5.2 Hz), 2.10 (s, 3H), 1.67-1.76 (m, 8H), 1.56 (m, 2H).

### Example 35

2-(2-Methyl-3-(2-(pyrrolidin-1-ylsulfonyl)benzyl)-5,6,7,8-tetrahydrocyclohepta[b]pyrrol-1(4H)-yl)acetic acid (I-22). ¹H NMR (400 MHz, CDCl₃) δ (ppm): 7.96 (d, 1H, J=7.6 Hz), 7.38 (t, 1H, J=7.6 Hz), 7.26 (t, 1H, J=7.6 Hz), 7.03 (d, 1H, J=7.6 Hz), 4.62 (s, 2H), 4.16 (s, 2H), 3.36 (t, 4H, J=6.4 Hz), 2.61 (t, 2H, J=5.2 Hz), 2.28 (t, 2H, J=4.8 Hz), 2.04 (s, 3H), 1.92-1.95 (m, 4H), 1.69-1.80 (m, 4H), 1.54 (m, 2H).

### Example 36

Preparation of 2-(2-methyl-3-(2-(morpholinosulfonyl)benzyl)-5,6-dihydrocyclopenta[b]pyrrol-1(4H)-yl)acetic acid (I-23). The compound was synthesized in the same fashion as 2-(2-methyl-3-(4-(pyrrolidin-1-ylsulfonyl)benzyl)-5,6-dihydrocyclopenta[b]pyrrol-1(4H)-yl)acetic acid except 2-(morpholinosulfonyl)benzaldehyde was used in the coupling instead of 4-(pyrrolidin-1-ylsulfonyl)benzaldehyde. ¹H NMR (400 MHz, CDCl₃) δ (ppm): 7.94 (d, 1H, J=8.0 Hz), 7.46 (t, 1H, J=8.0 Hz), 7.31 (t, 1H, J=8.0 Hz), 7.29 (m, 1H), 4.54 (s, 2H), 4.15 (s, 2H), 3.69 (m, 4H), 3.15 (m, 4H), 2.62 (m, 2H), 2.30 (m, 4H), 2.08 (s, 3H).

### Example 37

Preparation of 2-(2-methyl-3-(2-(phenylsulfonyl)benzyl)-5,6,7,8-tetrahydrocyclohepta[b]pyrrol-1(4H)-yl)acetic acid (I-24) The title compound was synthesized in the same fashion as 2-(2-methyl-3-(2-(morpholinosulfonyl)benzyl)-5,6,7,8-tetrahydrocyclohepta[b]pyrrol-1(4H)-yl)acetic acid except 2-(phenylsulfonyl)benzaldehyde was used in the coupling instead of 2-(morpholinosulfonyl)benzaldehyde. ¹H NMR (400 MHz, CDCl₃) δ (ppm): 8.30 (d, 1H, J=7.6 Hz), 7.91-7.89 (m, 2H), 7.57-7.37 (m, 5H), 6.94 (d, 1H, J=7.6 Hz), 4.54 (s, 2H), 3.85 (s, 2H), 2.52 (m, 2H), 1.81 (m, 5H), 1.66-1.62 (m, br, 4H), 1.33 (m, 2H).

### Example 38

### Preparation of Ethyl 2-(2,6,6-trimethyl-3-(2-(morpholinosulfonyl)benzyl)-4,5,6,7-tetrahydro-1H-indol-1-yl)acetate (I-74) and 2-(2,6,6-trimethyl-3-(2-(morpholinosulfonyl)benzyl)-4,5,6,7-tetrahydro-1H-indol-1-yl)acetic acid (I-26)

### Example 38A

Preparation of Ethyl 2-(2,6,6-trimethyl-4,5,6,7-tetrahydro-1H-indol-1-yl)acetate. To a stirred solution of ethyl 2-(2,6,6-trimethyl-4-oxo-4,5,6,7-tetrahydro-1H-indol-1-yl)acetate (0.7 g, 2.66 mmol) in THF (12 ml) was added Borane-THF (2.66 ml, 2.66 mmol) at room temperature, the mixture was stirred for 2 hours, tlc (EtOAc/Hex, 20%) checked that the reaction is done, EtOH was added to destroy residue diborane, water was added, extracted with ether, combine extracts were washed with brine, dried with sodium sulfate, filtered and concentrated to leave a liquid 0.31 g, 1.243 mmol, yield 46.8%. ¹H NMR (400 MHz, CDCl₃) δ (ppm): 5.72 (s, 1H), 4.41 (s, 2H), 4.21 (q, 2H, J=7.2 Hz), 2.45 (t, 2H, J=6.4 Hz), 2.19 (s, 2H), 2.16 (s, 3H), 1.47 (t, 2H, J=6.0 Hz), 1.27-1.24 (m, 3H), 0.99 (s, 6H).

### Example 38B

Preparation of Ethyl 2-(2,6,6-trimethyl-3-(2-(morpholinosulfonyl)benzyl)-4,5,6,7-tetrahydro-1H-indol-1-yl)acetate (I-74). To a flame-dried vial was charged with dichloromethane (5 ml) and trimethylsilyl trifluoromethanesulfonate (0.077 ml, 0.425 mmol) was added at 0° C., followed by a solution of ethyl 2-(2,6,6-trimethyl-4,5,6,7-tetrahydro-1H-indol-1-yl)acetate (53 mg, 0.213 mmol) and 2-(morpholinosulfonyl)benzaldehyde (81 mg, 0.319 mmol) in dichloromethane (5 ml) (cannulated), the mix was stirred at 0° C. for 15 min, neat triethylsilane (0.135 ml, 0.850 mmol) was added slowly, stirred for 30 min at 0° C., then slowly warmed to r.t. tlc checked that reaction was done, quenched with sat. NaHCO3, extracted with CH₂Cl₂. Combined CH₂Cl₂ was dried with Na₂SO₄, filtered and concentrated, the residue was chromatographed with Biotage, eluted with 5-50% EtOAc and Hexane, to give 47 mg, 0.096 mmol semisolid, yield 45.3%. ¹H NMR (400 MHz, CDCl₃) δ (ppm): 7.95 (d, 1H, J=8.0 Hz), 7.41 (t, 1H, J=7.6 Hz), 7.23 (t, 1H, J=7.2 Hz), 7.09 (d, 1H, J=8.0 Hz), 4.47 (s, 2H), 4.22 (q, 2H, J=7.2 Hz), 4.15 (s, 2H), 3.74 (m, 4H), 3.23 (m, 4H), 2.23 (m, 2H), 2.17 (m, 2H), 2.03 (m, 3H), 1.43 (t, 2H, J=6.4 Hz), 1.30-1.24 (m, 3H), 0.98 (s, 6H).

### Example 38C

Preparation of 2-(2,6,6-trimethyl-3-(2-(morpholinosulfonyl)benzyl)-4,5,6,7-tetrahydro-1H-indol-1-yl)acetic acid (I-26). Ethyl 2-(2,6,6-trimethyl-3-(2-(morpholinosulfonyl)benzyl)-4,5,6,7-tetrahydro-1H-indol-1-yl)acetate (40 mg, 0.082 mmol) in THF (2 ml), MeOH (0.533 ml) and Water (0.533 ml) was stirred, lithium hydroxide (3.92 mg, 0.164 mmol) was added, stirred at 25° C. for two hours, tlc checked that starting material was gone, remove solvent, water was added, followed by 1 N HCl, remove solvent, MeOH was added, purified by prep-HPLC to give 2-(2,6,6-trimethyl-3-(2-(morpholinosulfonyl)benzyl)-4,5,6,7-tetrahydro-1H-indol-1-yl)acetic acid (27.1 mg, 0.059 mmol, 71.9% yield). ¹H NMR (400 MHz, CDCl₃) δ (ppm): 7.94 (d, 1H, J=7.6 Hz), 7.40 (t, 1H, J=7.2 Hz), 7.28 (t, 1H, J=7.6 Hz), 7.07 (d, 1H, J=7.6 Hz), 4.48 (s, 2H), 4.14 (s, 2H), 3.74-3.72 (m, 4H), 3.46 (s, 1H), 3.23-3.20 (m, 4H), 2.22 (s, 2H), 2.16 (t, 2H, J=5.6 Hz), 2.02 (s, 3H), 1.42 (t, 2H, J=6.0 Hz), 0.97 (s, 6H).

### Example 39

Preparation of 2-(2,5,5-trimethyl-3-(3-(morpholinosulfonyl)benzyl)-4,5,6,7-tetrahydro-1H-indol-1-yl)acetic acid (I-27. The title compound was synthesized in the same fashion as I-17 except 3-(morpholinosulfonyl)benzaldehyde was used instead of 2-(morpholinosulfonyl)benzaldehyde. ¹H NMR (400 MHz, CDCl₃) δ (ppm): 7.52 (d, 1H, J=7.2 Hz), 7.37-7.45 (m, 3H), 4.52 (s, 2H), 3.79 (s, 2H), 3.71 (t, 4H, J=4.4 Hz), 2.92 (t, 4H, J=4.4 Hz), 2.42 (t, 2H, J=6.0 Hz), 2.09 (s, 3H), 2.03 (s, 2H), 1.53 (t, 2H), 0.91 (s, 6H).

### Example 40

Preparation of (R)-2-(2,5,5-trimethyl-3-(2-(morpholinosulfonyl)benzyl)-4,5,6,7-tetrahydro-1H-indol-1-yl)propanoic acid (I-35). Synthesis was carried out as in I-17 except (R)-ethyl 2-bromopropanoate was used instead of ethyl 2-aminoacetate. ¹H NMR (400 MHz, CDCl₃) δ (ppm): 7.97 (d, 1H, J=7.2 Hz), 7.41 (app t, 1H, J=7.6 Hz), 7.26-7.30 (m, 1H), 7.02 (d, 1H, J=7.6 Hz), 4.90 (q, 1H, J=7.6 Hz), 4.11 (s, 2H), 3.74 (m, 4H), 3.24 (m, 4H), 2.48-2.56 (m, 2H), 2.07 (s, 3H), 1.98 (s, 2H), 1.70 (d, 3H, J=7.2 Hz), 1.53-1.57 (m, 2H), 0.90 (s, 6H, two shifts isochronous).

### Example 41

Preparation of (S)-2-(2,5,5-trimethyl-3-(2-(morpholinosulfonyl)benzyl)-4,5,6,7-tetrahydro-1H-indol-1-yl)propanoic acid (I-34). Synthesis was carried out as in I-17 except (S)-ethyl 2-bromopropanoate was used instead of ethyl 2-aminoacetate. ¹H NMR (400 MHz, CDCl₃) δ (ppm): 7.96 (d, 1H, J=7.6 Hz), 7.41 (dd, 1H, J=8.0, J=7.6 Hz), 7.26-7.31 (m, 1H), 7.02 (d, 1H, J=7.6 Hz), 4.91 (q, 1H, J=6.8 Hz), 4.11 (s, 2H), 3.73-3.75 (m, 4H), 3.22-3.25 (m, 4H), 2.46-2.58 (m, 2H), 2.07 (s, 3H), 1.98 (s, 2H), 1.71 (d, 3H, J=7.2 Hz), 1.54-1.57 (m, 2H), 1.03 (s, 6H, two shifts isochronous).

### Example 42

### Preparation of 2-(2,7,7-trimethyl-3-(2-(morpholinosulfonyl)benzyl)-4,5,6,7-tetrahydro-1H-indol-1-yl)acetic acid (I-36) and Ethyl 2-(2,7,7-trimethyl-3-(2-(morpholinosulfonyl)benzyl)-4,5,6,7-tetrahydro-1H-indol-1-yl)acetate (I-61)

### Example 42A

Preparation of 2,2-dimethyl-6-(2-oxopropyl)cyclohexanone Sodium hydride (0.440 g, 17.43 mmol) was added to toluene (20 ml) at r.t. 2,2-dimethylcyclohexanone (2 g, 15.85 mmol) was added, stirred for 10 minutes, then heated at 40° C. for 3 hours, 3-chloro-2-(methoxymethoxy)prop-1-ene (2.268 ml, 17.43 mmol) was added dropwise and heated at 120° C. for 3 hours, cooled to r.t. quenched with MeOH (2.000 ml) and the solvent was washed with water, extracted with DCM, combined extracts were washed with brine, dried with Na₂SO₄, filtered and concentrated, to this residue was added 5 ml 1% H₂SO₄ and 5 ml dioxane, heated at 60° C. for one hour, the product was extracted with ether, combined extracts was dried with Na₂SO₄, filtered and concentrated, the residue was purified by Biotage, to give liquid 2,2-dimethyl-6-(2-oxopropyl)cyclohexanone (0.42 g, 2.304 mmol, 14.54% yield). ¹H NMR (400 MHz, CDCl₃) δ (ppm): 3.19-3.27 (m, 1H), 2.91 (dd, 1H, J=17.2 Hz, J=17.2 Hz), 2.20 (s, 3H), 2.10 (dd, 1H, J=4.8 Hz, J=4.8 Hz), 1.97-2.06 (m, 1H), 1.86-1.93 (m, 1H), 1.75-1.80 (m, 1H), 1.63-1.68 (m, 1H), 1.49-1.55 (m, 1H), 1.27-1.38 (m, 1H), 1.22 (s, 3H), 1.02 (s, 3H).

### Example 42B

Preparation of Ethyl 2-(2,7,7-trimethyl-4,5,6,7-tetrahydro-1H-indol-1-yl)acetate 2,2-dimethyl-6-(2-oxopropyl)cyclohexanone (0.42 g, 2.304 mmol) in dichloromethane (3 ml) was stirred, ethyl 2-aminoacetate hydrochloride (0.482 g, 3.46 mmol) was added, followed by sodium hydrogencarbonate (0.387 g, 4.61 mmol), the mixture was stirred at 55° C. for 75 hours, TLC showed that reaction wasn't done. dichloromethane was added, washed with water, brine, dried with NaHSO₄, filtered and concentrated, the residue was chromatographed via Biotage using EtOAc/Hexane 5-10%, to give 0.25 g, 1.003 mmol, yield 43.5%. ¹H NMR (400 MHz, CDCl₃) δ (ppm): 5.72 (s, 1H), 4.65 (s, 2H), 4.27 (q, 2H, J=7.2 Hz), 2.47 (t, 2H, J=6.0 Hz), 2.14 (s, 3H), 1.70-1.75 (m, 2H), 1.62-1.65 (m, 2H), 1.31 (t, 3H, J=7.2 Hz), 1.27 (s, 6H).

### Example 42C

Preparation of Ethyl 2-(2,7,7-trimethyl-3-(2-(morpholinosulfonyl)benzyl)-4,5,6,7-tetrahydro-1H-indol-1-yl)acetate (I-61) To a flame-dried vial was charged with dichloromethane (5 ml) and trimethylsilyl trifluoromethanesulfonate (0.077 ml, 0.425 mmol) was added at 0° C., followed by a solution of ethyl 2-(2,7,7-trimethyl-4,5,6,7-tetrahydro-1H-indol-1-yl)acetate (53 mg, 0.213 mmol) and 2-(morpholinosulfonyl)benzaldehyde (81 mg, 0.319 mmol) in dichloromethane (5 ml) (cannulated), the mix was stirred at 0° C. for 15 min, neat triethylsilane (0.102 ml, 0.638 mmol) was added slowly, stirred for 30 min at 0° C., then slowly warmed to r.t. Checked by tlc to ensure that reaction was done, quenched with sat. NaHCO₃, extracted with dichloromethane. Combined organics were dried with Na2SO4, filtered and concentrated, the residue was chromatographed via Biotage, eluting with 5-50% EtOAc and Hexane, to give ethyl 2-(2,7,7-trimethyl-3-(2-(morpholinosulfonyl)benzyl)-4,5,6,7-tetrahydro-1H-indol-1-yl)acetate (43 mg, 0.088 mmol, 41.4% yield). ¹H NMR (400 MHz, CDCl₃) δ (ppm): 7.97 (d, 1H, J=8.0 Hz), 7.40-7.43 (m, 1H), 7.26-7.30 (m, 1H), 7.10 (d, 1H, J=7.6 Hz), 4.68 (s, 2H), 4.23 (q, 2H, J=7.2 Hz), 4.12 (s, 2H), 3.73 (m, 4H), 3.22 (m, 4H), 2.15 (m, 2H), 1.97 (s, 3H), 1.29 (t, 3H, J=6.8 Hz), 1.59-1.65 (m, 4H), 1.24 (s, 6H).

### Example 42D

Preparation of 2-(2,7,7-trimethyl-3-(2-(morpholinosulfonyl)benzyl)-4,5,6,7-tetrahydro-1H-indol-1-yl)acetic acid (I-36) Ethyl 2-(2,7,7-trimethyl-3-(2-(morpholinosulfonyl)benzyl)-4,5,6,7-tetrahydro-1H-indol-1-yl)acetate (43 mg, 0.088 mmol) in THF (2 ml), MeOH (0.533 ml) and Water (0.533 ml) was stirred, lithium hydroxide (4.21 mg, 0.176 mmol) was added, stirred at 25° C. for two hours, TLC was checked to determine that starting material was gone, remove solvent, water was added, followed by 1 N HCl, remove solvent, MeOH was added, purified by prep-HPLC to give a pink solid, 24 mg, 0.052 mmol, yield 59.2%. ¹H NMR (400 MHz, CDCl₃) δ (ppm): 7.96 (d, 1H, J=8.0 Hz), 7.45 (t, 1H, J=7.2 Hz), 7.29 (t, 1H, J=7.6 Hz), 7.06 (d, 1H, J=7.2 Hz), 4.76 (s, 2H), 4.13 (s, 2H), 3.72-3.75 (m, 4H), 3.21-3.24 (m, 4H), 2.16 (t, 2H, J=5.6 Hz), 1.99 (s, 3H), 1.60-1.67 (m, 4H), 1.29 (s, 6H).

### Example 43

### Preparation of Ethyl 2-(2,6,6-trimethyl-4-oxo-3-(2-(phenylsulfonyl)benzyl)-4,5,6,7-tetrahydro-1H-indol-1-yl)acetate (I-63) and 2-(2,6,6-trimethyl-4-oxo-3-(2-(phenylsulfonyl)benzyl)-4,5,6,7-tetrahydro-1H-indol-1-yl)acetic acid (I-48)

### Example 43A

Preparation of 2-(phenylsulfonyl)benzaldehyde. To a flame dried vial was charged with 2-fluorobenzaldehyde (0.842 ml, 8.06 mmol), Sodium benzenesulfinate (1.323 g, 8.06 mmol) and DMSO (5 ml) under nitrogen, the mixture was heated at 130° C. for 16 hours, then poured into ice water and extracted with DCM, combined DCM was dried with sodium sulfate and concentrated, the residue was chromatographed with Biotage, 5-50-70%, to give white solid 2-(phenylsulfonyl)benzaldehyde (1.5 g, 6.09 mmol, 76% yield). ¹H NMR (400 MHz, CDCl₃) δ (ppm): 17.85 (s, 1H), 8.21 (dd, 1H, J=7.2 Hz, J=1.6 Hz), 8.03 (m, 1H), 7.92-7.89 (m, 2H), 7.79-7.16 (m, 2H), 7.63-7.25 (m, 3H).

### Example 43B

Preparation of 3-(hydroxy(2-(phenylsulfonyl)phenyl)methyl)-2,6,6-trimethyl-6,7-dihydro-1H-indol-4(5H)-one. To a solution of 2,6,6-trimethyl-6,7-dihydro-1H-indol-4(5H)-one (100 mg, 0.564 mmol) in Methanol (5 ml) at 0° C. was added solid 2-(phenylsulfonyl)benzaldehyde (139 mg, 0.564 mmol). At 0° C. a 3M solution of sodium hydroxide (0.564 ml, 1.693 mmol) was added. The reaction was stirred at 0° C. for 30 minutes, after which all starting material remained as determined by the LCMS. Continued stirring at 55° C. overnight. The next day, LCMS indicated that the reaction had approached about 60% conversion with the remainder as starting material. The reaction was diluted in water, extracted with EtOAc, combined extracts was dried with Na₂SO₄, filtered and concentrated. Chromatographed via Biotage 5-50-70% to give an off-white solid, 3-(hydroxy(2-(phenylsulfonyl)phenyl)methyl)-2,6,6-trimethyl-6,7-dihydro-1H-indol-4(5H)-one (50 mg, 0.118 mmol, 20.92% yield). ¹H NMR (400 MHz, CDCl₃) δ (ppm): 9.06 (b, 1H), 8.18-8.16 (m, 1H), 7.97-7.94 (m, 2H), 7.52-7.25 (m, 6H), 6.97 (d, 1H, J=9.6 Hz), 6.77 (d, 1H, J=9.6 Hz), 2.57 (d, 1H, J=16.4 Hz), 2.52 (d, 1H, J=16.4 Hz), 2.40 (d, 1H, J=16.4 Hz), 2.32 (d, 1H, J=16.4 Hz), 1.76 (s, 3H), 1.09 (s, 3H), 1.04 (s, 3H).

### Example 43C

Preparation of 2,6,6-trimethyl-3-(2-(phenylsulfonyl)benzyl)-6,7-dihydro-1H-indol-4(5H)-one. To a solution of the 3-(hydroxy(2-(phenylsulfonyl)phenyl)methyl)-2,6,6-trimethyl-6,7-dihydro-1H-indol-4(5H)-one (50 mg, 0.118 mmol) in DCM (2 ml) was added trimethylsilyl trifluoromethanesulfonate (0.021 ml, 0.118 mmol) at 0° C. The reaction was stirred at 0° C. for 5 minutes, after which triethylsilane (0.021 ml, 0.130 mmol) was added. After 15 minutes, the reaction was monitored by LCMS and determined to be complete. The reaction was quenched by the addition of saturated sodium bicarbonate solution, extracted with DCM (3×10 ml), dried (sodium sulfate), filtered and concentrated to afford 2,6,6-trimethyl-3-(2-(phenylsulfonyl)benzyl)-6,7-dihydro-1H-indol-4(5H)-one (42 mg, 0.103 mmol, 87% yield). ¹H NMR (400 MHz, CDCl₃) δ (ppm): 8.24 (d, 1H, J=7.6 Hz), 7.93-7.91 (m, 2H), 7.82 (b, 1H), 7.56-7.49 (m, 3H), 7.38-7.30 (m, 2H), 6.97 (d, 1H, J=7.6 Hz), 4.29 (s, 2H), 2.60 (s, 2H), 2.23 (s, 2H), 1.88 (s, 3H), 1.09 (s, 6H).

### Example 43D

Preparation of Ethyl 2-(2,6,6-trimethyl-4-oxo-3-(2-(phenylsulfonyl)benzyl)-4,5,6,7-tetrahydro-1H-indol-1-yl)acetate (I-63). To a solution of 2,6,6-trimethyl-3-(2-(phenylsulfonyl)benzyl)-6,7-dihydro-1H-indol-4(5H)-one (23 mg, 0.056 mmol) in Acetonitrile (1 ml) in a microwave tube was added ethyl 2-bromoacetate (0.013 ml, 0.113 mmol), potassium carbonate (11.70 mg, 0.085 mmol), and potassium iodide (1.874 mg, 0.011 mmol). The reaction was sealed and heated to 75° C. After 16 hours, the reaction was shown to be ˜90% complete by LCMS. The reaction was cooled to room temperature, diluted with saturated ammonium chloride solution, extracted with dichloromethane (3×5 ml), dried (sodium sulfate), filtered, and concentrated to a gold residue which was purified on silica gel to give ethyl 2-(2,6,6-trimethyl-4-oxo-3-(2-(phenylsulfonyl)benzyl)-4,5,6,7-tetrahydro-1H-indol-1-yl)acetate (20 mg, 0.041 mmol, 71.8% yield). ¹H NMR (400 MHz, CDCl₃) δ (ppm): 8.25-8.22 (m, 1H), 7.93-7.91 (m, 2H), 7.56-7.48 (m, 3H), 7.35-7.31 (m, 2H), 6.944-6.923 (m, 1H), 4.48 (s, 2H), 4.31 (s, 2H), 4.22 (q, 2H, J=7.2 Hz), 2.52 (s, 2H), 2.24 (s, 2H), 1.83 (s, 3H), 1.27 (t, 3H, J=7.2 Hz), 1.09 (s, 6H).

### Example 43E

Preparation of 2-(2,6,6-trimethyl-4-oxo-3-(2-(phenylsulfonyl)benzyl)-4,5,6,7-tetrahydro-1H-indol-1-yl)acetic acid (I-48). To a solution of ethyl 2-(2,6,6-trimethyl-4-oxo-3-(2-(phenylsulfonyl)benzyl)-4,5,6,7-tetrahydro-1H-indol-1-yl)acetate (20 mg, 0.041 mmol) in THF (1 ml) and water (0.50 ml) was added sodium hydroxide (0.081 ml, 0.081 mmol). The reaction was stirred at r.t. for 30 minutes, after which analysis by LCMS indicated full conversion to the product. The mixture was purified by prep-HPLC to give 2-(2,6,6-trimethyl-4-oxo-3-(2-(phenylsulfonyl)benzyl)-4,5,6,7-tetrahydro-1H-indol-1-yl)acetic acid (3.5 mg, 7.52 μmol, 18.55% yield). ¹H NMR (400 MHz, CDCl₃) δ (ppm): 8.21 (dd, 1H, J=7.6 Hz, J=1.6 Hz), 7.92-7.89 (m, 2H), 7.58-7.48 (m, 3H), 7.35-7.31 (m, 2H), 6.89 (dd, 1H, J=7.6 Hz, J=1.6 Hz), 4.53 (s, 2H), 4.29 (s, 2H), 2.54 (s, 2H), 2.28 (s, 2H), 1.83 (s, 3H), 1.08 (s, 6H).

### Example 44

### Preparation of ethyl 2-(2,6,6-trimethyl-4-oxo-3-(2-(pyrrolidin-1-ylsulfonyl)benzyl)-4,5,6,7-tetrahydro-1H-indol-1-yl)acetate (I-51) and 2-(2,6,6-trimethyl-4-oxo-3-(2-(pyrrolidin-1-ylsulfonyl)benzyl)-4,5,6,7-tetrahydro-1H-indol-1-yl)acetic acid (I-43)

### Example 44A

Preparation of 1-(phenylsulfonyl)pyrrolidine. To a solution of benzenesulfonyl chloride (7.30 ml, 56.6 mmol) in acetone (100 ml) was added neat pyrrolidine (9.36 ml, 113 mmol). The reaction was stirred at room temperature for 4 hours, after which it was concentrated, extracted w/ dichloromethane (3×75 mL), dried (sodium sulfate), filtered and concentrated to an off-white crystalline solid 1-(phenylsulfonyl)pyrrolidine (10.3 g, 48.8 mmol, 86% yield). ¹H NMR (400 MHz, CDCl₃) δ (ppm): 7.83 (m, 2H), 7.50-7.61 (m, 3H), 3.25 (m, 4H), 1.75 (m, 4H).

### Example 44B

Preparation of sodium hydroxy(2-(pyrrolidin-1-ylsulfonyl)phenyl)methanesulfonate. To a solution of 1-(phenylsulfonyl)pyrrolidine (4.88 g, 23.10 mmol) in THF (47.0 mL) at 0° C. was added a hexane solution of BuLi (13.93 mL, 27.7 mmol). The reaction was stirred at 0° C. for 30 minutes, after which a solution of morpholine-4-carbaldehyde (2.80 mL, 27.7 mmol) in THF (28 mL) was added. The reaction was stirred at 0° C. for 1 hour, after which an aliquot was removed and quenched with water, then concentrated to dryness. The reaction was allowed to warm to room temperature and stirred for 14 hours (overnight), then quenched by addition of water (40 mL) and the organic layer was separated. The aqueous layer was treated with solid NaCl, then extraction was performed with diethyl ether (3×75 mL). The combined organic extracts were washed with saturated sodium chloride solution (2×100 mL), dried (sodium sulfate), filtered and concentrated to a dark brown residue, which was shown by ¹H NMR to be a 2:1 mixture of 2-(pyrrolidin-1-ylsulfonyl)benzaldehyde (4.90 g, 13.7 mmol, 59% yield) to 1-(phenylsulfonyl)pyrrolidine. The solid mixture was dissolved in Et₂O (20 mL) to which a solution of sodium bisulfite (2.86 g, 27.4 mmol) in water (20 mL) was added. The solution was stirred at room temperature for 2 hours, leading to the formation of an off-white precipitate which was washed in ether then hexanes. The first crop afforded 3.33 g of analytically pure bisulfite adduct, and the second crop (refiltered material) afforded 293 mg. The off white solid, sodium hydroxy(2-(pyrrolidin-1-ylsulfonyl)phenyl)methanesulfonate (3.623 g, 10.55 mmol, 77% yield) was dried in vacuo. ¹H NMR (400 MHz, D₂O) δ (ppm): 7.92 (d, 1H, J=8.0 Hz), 7.83 (d, 1H, J=8.0 Hz), 7.61 (t, 1H, J=7.8 Hz), 7.49 (t, 1H, J=7.8 Hz), 6.33 (s, 1H), 3.08-3.15 (m, 4H), 1.70 (m, 4H).

### Example 44C

Preparation of 2-(pyrrolidin-1-ylsulfonyl)benzaldehyde. To a solution of sodium hydroxy(2-(pyrrolidin-1-ylsulfonyl)phenyl)methanesulfonate (2.79 g, 8.13 mmol) in THF (16 mL) and water (16 mL) was added sodium carbonate (1.72 g, 16.3 mmol). The reaction was heated to 75° C. for 1.5 hours, then cooled to room temperature, concentrated to ˜½ volume by removing the THF in vacuo, extracted with dichloromethane (3×50 mL), dried (sodium sulfate), filtered, and concentrated to a gold oil which solidified on standing affording 2-(pyrrolidin-1-ylsulfonyl)benzaldehyde (1.65 g, 6.90 mmol, 85% yield). ¹H NMR (400 MHz, CDCl₃) δ (ppm): 10.9 (s, 1H), 8.07 (m, 1H), 7.98 (m, 1H), 7.72 (m, 2H), 3.29 (t, 4H, J=6.4 Hz), 1.87 (m, 4H).

### Example 44D

Preparation of 3-(hydroxy(2-(pyrrolidin-1-ylsulfonyl)phenyl)methyl)-2,6,6-trimethyl-6,7-dihydro-1H-indol-4(5H)-one. To a solution of 2,6,6-trimethyl-6,7-dihydro-1H-indol-4(5H)-one (920 mg, 5.19 mmol) in methanol (10.4 mL) was added solid 2-(pyrrolidin-1-ylsulfonyl)benzaldehyde (1.25 g, 5.22 mmol). At room temperature, a 3M aqueous solution of sodium hydroxide (5.19 mL, 15.6 mmol) was added. The reaction was stirred at 55° C. for 12 hours, after which the reaction was diluted with water (8 mL), leading to the formation of an off-white precipitate. This solid was collected and washed with water, and was about 90% pure by NMR. This solid was purified on silica gel (Luknova 80 g, 20 mL/min) using 1 to 7% MeCN:MeOH 7:1 in dichloromethane over 70 minutes to afford 3-(hydroxy(2-(pyrrolidin-1-ylsulfonyl)phenyl)methyl)-2,6,6-trimethyl-6,7-dihydro-1H-indol-4(5H)-one (1.25 g, 3.00 mmol, 58% yield) as a solid. ¹H NMR (400 MHz, CDCl₃) δ (ppm): 8.90 (br s, 1H), 7.96 (dd, 1H, J=8.0 Hz, J=0.8 Hz), 7.47 (dd, 1H, J=7.6 Hz, J=0.8 Hz), 7.41 (app. td, 1H, J=8.0 Hz, J=0.8 Hz), 7.31 (ddd, 1H, J=8.0 Hz, J=7.6 Hz, J=1.2 Hz), 7.27 (d, 1H, J=9.6 Hz), 6.69 (d, 1H, J=9.6 Hz), 3.47-3.53 (m, 2H), 3.25-3.30 (m, 2H), 2.64 (d, 1H, J=16.0 Hz), 2.59 (d, 1H, J=16.0 Hz), 2.47 (d, 1H, J=16.8 Hz), 2.40 (d, 1H, J=16.4 Hz), 1.86-1.88 (m, 7H), 1.14 (s, 3H), 1.08 (s, 3H).

### Example 44E

Preparation of 2,6,6-trimethyl-3-(2-(pyrrolidin-1-ylsulfonyl)benzyl)-6,7-dihydro-1H-indol-4(5H)-one. To a 0° C. solution of 3-(hydroxy(2-(pyrrolidin-1-ylsulfonyl)phenyl)methyl)-2,6,6-trimethyl-6,7-dihydro-1H-indol-4(5H)-one (30.0 mg, 0.0720 mmol) in dichloromethane (2 ml) was added trimethylsilyl trifluoromethanesulfonate (13.0 μl, 0.0720 mmol). The reaction turned yellow immediately. After 5 minutes, triethylsilane (12.7 μl, 0.0790 mmol) was added. The reaction was stirred at 0° C., and was complete after 50 minutes. Saturated sodium bicarbonate solution (5 mL) was added to quench the reaction, and the reaction was extracted with dichloromethane (3×30 mL), dried (sodium sulfate), filtered, and concentrated to a white solid identified as 2,6,6-trimethyl-3-(2-(pyrrolidin-1-ylsulfonyl)benzyl)-6,7-dihydro-1H-indol-4(5H)-one (27.2 mg, 0.0680 mmol, 94% yield). No purification was necessary. ¹H NMR (400 MHz, CDCl₃) δ (ppm): 8.16 (br s, 1H), 7.99 (dd, 1H, J=8.0 Hz, J=1.6 Hz), 7.32 (app. td, 1H, J=7.6 Hz, J=1.6 Hz), 7.24 (ddd, 1H, J=8.0 Hz, J=7.6 Hz, J=1.6 Hz), 6.98 (dd, 1H, J=7.6 Hz, J=0.8 Hz), 4.51 (s, 2H), 3.39 (m, 4H), 2.65 (s, 2H), 2.28 (s, 2H), 2.03 (s, 3H), 1.93 (m, 4H), 1.12 (s, 6H).

### Example 44F

Preparation of ethyl 2-(2,6,6-trimethyl-4-oxo-3-(2-(pyrrolidin-1-ylsulfonyl)benzyl)-4,5,6,7-tetrahydro-1H-indol-1-yl)acetate (I-51). To a solution of 2,6,6-trimethyl-3-(2-(pyrrolidin-1-ylsulfonyl)benzyl)-6,7-dihydro-1H-indol-4(5H)-one (27.2 mg, 0.0680 mmol) in acetonitrile (1.6 mL) in a sealed tube was added ethyl bromoacetate (0.0150 ml, 0.136 mmol), potassium carbonate (14.1 mg, 0.102 mmol), and potassium iodide (2.30 mg, 0.0140 mmol). The reaction was sealed and heated to 85° C. After 16 hours, the reaction was determined to be complete by LCMS analysis, and was diluted with saturated ammonium chloride solution and extracted with dichloromethane (3×30 mL), dried (sodium sulfate), filtered, and concentrated to a yellow residue. Purification by column chromatography on silica gel (Luknova 25 g, 20 mL/min) using 1 to 7% 7:1 MeCN:MeOH in DCM over 70 minutes furnished ethyl 2-(2,6,6-trimethyl-4-oxo-3-(2-(pyrrolidin-1-ylsulfonyl)benzyl)-4,5,6,7-tetrahydro-1H-indol-1-yl)acetate (27.8 mg, 0.0570 mmol, 84% yield). ¹H NMR (400 MHz, CDCl₃) δ (ppm): 7.99 (dd, 1H, J=7.6 Hz, J=1.2 Hz), 7.31 (app. td, 1H, J=7.6 Hz, J=1.2 Hz), 7.23 (app. t, 1H, J=7.2 Hz), 6.94 (d, 1H, J=7.2 Hz), 4.54 (s, 4H, two shifts isochronous), 4.25 (q, 2H, J=7.2 Hz), 3.38-3.41 (m, 4H), 2.57 (s, 2H), 2.29 (s, 2H), 1.98 (s, 3H), 1.92-1.96 (m, 4H), 1.29 (t, 3H, J=7.2 Hz), 1.13 (s, 6H).

### Example 44G

Preparation of 2-(2,6,6-trimethyl-4-oxo-3-(2-(pyrrolidin-1-ylsulfonyl)benzyl)-4,5,6,7-tetrahydro-1H-indol-1-yl)acetic acid (I-43). To a solution of ethyl 2-(2,6,6-trimethyl-4-oxo-3-(2-(pyrrolidin-1-ylsulfonyl)benzyl)-4,5,6,7-tetrahydro-1H-indol-1-yl)acetate (27.8 mg, 0.0570 mmol) in THF (0.7 mL) and water (0.6 mL) was added aqueous 1M sodium hydroxide solution (0.114 mL, 0.114 mmol). After stirring for 30 minutes, the reaction was quenched by the addition of 3M hydrochloric acid (0.038 mL), extracted with dichloromethane (3×30 mL), dried (sodium sulfate), filtered, and concentrated to a white solid 2-(2,6,6-trimethyl-4-oxo-3-(2-(pyrrolidin-1-ylsulfonyl)benzyl)-4,5,6,7-tetrahydro-1H-indol-1-yl)acetic acid (22.7 mg, 0.050 mmol, 87% yield).). ¹H NMR (400 MHz, CDCl₃) δ (ppm): 7.95 (dd, 1H, J=7.6 Hz, J=1.6 Hz), 7.30 (app. td, 1H, J=7.6 Hz, J=1.6 Hz), 7.22 (app. td, 1H, J=7.6 Hz, J=0.8 Hz), 6.92 (d, 1H, J=6.8 Hz), 4.54 (s, 2H), 4.50 (s, 2H), 3.36-3.39 (m, 4H), 2.56 (s, 2H), 2.27 (s, 2H), 1.98 (s, 3H), 1.91-1.94 (m, 4H), 1.09 (s, 6H).

### Example 45

### Preparation of ethyl 2-(2,6,6-trimethyl-3-(2-(morpholinosulfonyl)benzyl)-4-oxo-4,5,6,7-tetrahydro-1H-indol-1-yl)acetate (I-56) and 2-(2,6,6-trimethyl-3-(2-(morpholinosulfonyl)benzyl)-4-oxo-4,5,6,7-tetrahydro-1H-indol-1-yl)acetic acid (I-40)

### Example 45A

Preparation of 3-(hydroxy(2-(morpholinosulfonyl)phenyl)methyl)-2,6,6-trimethyl-6,7-dihydro-1H-indol-4(5H)-one. To a solution of 2,6,6-trimethyl-6,7-dihydro-1H-indol-4(5H)-one (512 mg, 2.89 mmol) in methanol (12 mL) at 0° C. was added solid 2-(morpholinosulfonyl)benzaldehyde (0.737 g, 2.89 mmol; purchased from Maybridge Inc.). At 0° C., a 1M aqueous solution of sodium hydroxide (2.89 mL, 8.67 mmol) was added. The reaction was stirred at 0° C. for 30 minutes, after which all starting material remained. The reaction was warmed 55° C. and stirred for 12 hours, after which the reaction was approximately 60% complete. The reaction was diluted in water, then allowed to stand for 1 hour. A white precipitate formed, which was filtered and washed with water, then dried to afford an off-white solid, 3-(hydroxy(2-(morpholinosulfonyl)phenyl)methyl)-2,6,6-trimethyl-6,7-dihydro-1H-indol-4(5H)-one (0.390 g, 0.902 mmol, 31.2% yield). ¹H NMR (400 MHz, CDCl₃) δ (ppm): 8.66 (br s, 1H), 7.92 (dd, 1H, J=8.0 Hz, J=1.2 Hz), 7.52 (dd, 1H, J=7.6 Hz, J=1.2 Hz), 7.45 (ddd, 1H, J=8.0 Hz, J=7.6 Hz, J=1.2 Hz), 7.34 (ddd, 1H, J=8.0 Hz, J=7.6 Hz, J=1.2 Hz), 7.26 (d, 1H, J=9.2 Hz), 6.66 (d, 1H, J=9.6 Hz), 3.71-3.78 (m, 4H), 3.30-3.38 (m, 2H), 3.12-3.17 (m, 2H), 2.65 (d, 1H, J=16.4 Hz), 2.60 (d, 1H, J=16.4 Hz), 2.48 (d, 1H, J=16.4 Hz), 2.42 (d, 1H, J=16.4 Hz), 1.91 (s, 3H), 1.15 (s, 3H), 1.10 (s, 3H).

### Example 45B

Preparation of 2,6,6-trimethyl-3-(2-(morpholinosulfonyl)benzyl)-6,7-dihydro-1H-indol-4(5H)-one. To a solution of 3-(hydroxy(2-(morpholinosulfonyl)phenyl)methyl)-2,6,6-trimethyl-6,7-dihydro-1H-indol-4(5H)-one (410 mg, 0.948 mmol) in dichloromethane (19.0 ml) was added trimethylsilyl trifluoromethanesulfonate (0.171 ml, 0.948 mmol) at 0° C. The reaction was stirred at 0° C. for 5 minutes, after which triethylsilane (0.167 ml, 1.04 mmol) was added. After 15 minutes, the reaction was complete, then quenched by the addition of saturated sodium bicarbonate solution, extracted with dichloromethane (3×30 mL), dried (sodium sulfate), filtered and concentrated to afford 2,6,6-trimethyl-3-(2-(morpholinosulfonyl)benzyl)-6,7-dihydro-1H-indol-4(5H)-one as a light tan solid. Used in the next step without further purification. ¹H NMR (400 MHz, CDCl₃) δ (ppm): 7.99 (br s, 1H), 7.97 (dd, 1H, J=7.6 Hz, J=1.2 Hz), 7.36 (ddd, 1H, J=8.0 Hz, J=7.6 Hz, J=1.2 Hz), 7.26 (m, 1H), 7.00 (dd, 1H, J=7.6 Hz, J=0.8 Hz), 4.51 (s, 2H), 3.80 (m, 4H), 3.28 (m, 4H), 2.66 (s, 2H), 2.28 (s, 2H), 2.06 (s, 3H), 1.12 (s, 6H).

### Example 45C

ethyl 2-(2,6,6-trimethyl-3-(2-(morpholinosulfonyl)benzyl)-4-oxo-4,5,6,7-tetrahydro-1H-indol-1-yl)acetate (I-56). To a solution of 2,6,6-trimethyl-3-(2-(morpholinosulfonyl)benzyl)-6,7-dihydro-1H-indol-4(5H)-one (395 mg, 0.948 mmol) in acetonitrile (11.9 ml) in a sealed tube was added ethyl bromoacetate (0.211 ml, 1.90 mmol), potassium carbonate (197 mg, 1.42 mmol), and potassium carbonate (31.5 mg, 0.190 mmol). The reaction heated to 75° C. After 16 hours, the reaction was shown to be ˜90% complete by LCMS. The reaction was cooled to room temperature, diluted with saturated ammonium chloride solution, extracted with dichloromethane (3×50 mL), dried (sodium sulfate), filtered, and concentrated to a gold residue which was purified on silica gel (Luknova 80 g, 20 mL/min) using 1 to 7% of 7:1 MeCN:MeOH in dichloromethane over 70 minutes affording 2-(2,6,6-trimethyl-3-(2-(morpholinosulfonyl)benzyl)-4-oxo-4,5,6,7-tetrahydro-1H-indol-1-yl)acetate (264 mg, 0.525 mmol, 55% yield) as a light tan solid. ¹H NMR (400 MHz, CDCl₃) δ (ppm): 7.96 (dd, 1H, J=7.6 Hz, J=1.2 Hz), 7.34 (ddd, 1H, J=7.6 Hz, J=7.2 Hz, J=1.2 Hz), 7.25 (m, 1H), 6.95 (d, 1H, J=8.0 Hz), 4.54 (s, 4H, two shifts isochronous), 4.24 (q, 2H, J=7.2 Hz), 3.80 (m, 4H), 3.27 (m, 4H), 2.57 (s, 2H), 2.29 (s, 2H), 1.99 (s, 3H), 1.30 (t, 3H, J=7.2 Hz), 1.13 (s, 6H).

### Example 45D

Preparation of 2-(2,6,6-trimethyl-3-(2-(morpholinosulfonyl)benzyl)-4-oxo-4,5,6,7-tetrahydro-1H-indol-1-yl)acetic acid (I-40). To a solution of ethyl 2-(2,6,6-trimethyl-3-(2-(morpholinosulfonyl)benzyl)-4-oxo-4,5,6,7-tetrahydro-1H-indol-1-yl)acetate (264 mg, 0.525 mmol) in THF (5.3 mL) and water (5.3 mL) was added aqueous 1M sodium hydroxide solution (1.05 ml, 1.05 mmol). The reaction was stirred at room temperature for 30 minutes, after which analysis by LCMS indicated full conversion to the product. The reaction was poured directly into a separatory funnel, washed with hexanes (1×50 mL), then dichloromethane (1×50 mL). The aqueous layer was neutralized by the addition of 3M aqueous hydrochloric acid (0.0350 mL), extracted with dichloromethane (3×50 mL), dried (sodium sulfate), filtered, and concentrated to a white solid 2-(2,6,6-trimethyl-3-(2-(morpholinosulfonyl)benzyl)-4-oxo-4,5,6,7-tetrahydro-1H-indol-1-yl)acetic acid (231 mg, 0.487 mmol, 93% yield). ¹H NMR (400 MHz, acetone-d6) δ (ppm): 7.92 (dd, 1H, J=7.6 Hz, J=1.2 Hz), 7.41 (ddd, 1H, J=8.0 Hz, J=7.6 Hz, J=1.6 Hz), 7.35 (ddd, 1H, J=8.0 Hz, J=7.6 Hz, J=1.2 Hz), 7.01 (dd, 1H, J=7.6 Hz, J=1.2 Hz), 4.79 (s, 2H), 4.54 (s, 2H), 3.75 (m, 4H), 3.21 (m, 4H), 2.71 (s, 2H), 2.20 (s, 2H), 2.05 (s, 3H), 1.11 (s, 6H).

### Example 46

### Preparation of ethyl 2-(3-(2-(N,N-dimethylsulfamoyl)benzyl)-2,6,6-trimethyl-4-oxo-4,5,6,7-tetrahydro-1H-indol-1-yl)acetate (I-68) and 24342-(N,N-dimethylsulfamoyl)benzyl)-2,6,6-trimethyl-4-oxo-4,5,6,7-tetrahydro-1H-indol-1-yl)acetic acid (I-52)

### Example 46A

Preparation of N,N-dimethylbenzenesulfonamide. To a sealed tube containing N-methylbenzenesulfonamide (5.10 g, 29.8 mmol) was added N,N-dimethylformamide dimethyl acetal (18.2 mL, 142 mmol). The reaction was sealed and heated to 100° C. for 12 hours, after which the reaction was cooled to room temperature then concentrated to afford N,N-dimethylbenzenesulfonamide (5.20 g, 28.1 mmol, 94% yield) as a clear viscous oil. ¹H NMR (400 MHz, CDCl₃) δ (ppm): 7.77-7.81 (m, 2H), 7.61 (tt, 1H, J=7.6 Hz, J=1.2 Hz), 7.55 (m, 2H), 2.70 (s, 6H).

### Example 46B

Preparation of 2-formyl-N,N-dimethylbenzenesulfonamide. To a flame-dried 250 mL flask was added N,N-dimethylbenzenesulfonamide (3.00 g, 16.2 mmol) followed by THF (54 mL). After cooling to 0° C., n-butyllithium (8.13 mL, 19.4 mmol) was added. Upon addition of n-BuLi, an initial green color was observed, which turned a dark reddish-brown upon completion of addition. The reaction was stirred at 0° C. for 30 minutes, after which N,N-dimethylformamide (1.51 mL, 19.4 mmol) was added. After 20 minutes, the reaction was quenched by the addition of saturated ammonium chloride (100 mL), extracted with dichloromethane (3×100 mL), dried (sodium sulfate), filtered, and concentrated to a clear residue. To this residue was added diethyl ether (27.0 mL) followed by a solution of sodium bisulfite (3.37 g, 32.4 mmol) dissolved in water (27.0 mL). This biphasic solution was stirred at room temperature for two hours, after which the layers were separated, and the water layer was washed with diethyl ether (2×50 mL). To this aqueous layer was added THF (60 ml) and a solution of sodium carbonate (4.28 g, 40.4 mmol) dissolved in water (100 ml). This solution was heated to 55° C. for two hours, after which it was extracted with dichloromethane (3×50 mL), dried (sodium sulfate), filtered, and concentrated to a clear colorless residue 2-formyl-N,N-dimethylbenzenesulfonamide (1.92 g, 9.00 mmol, 56% yield). ¹H NMR (400 MHz, CDCl₃) δ (ppm): 10.83 (s, 1H), 8.07-8.11 (m, 1H), 7.93-7.97 (m, 1H), 7.71-7.77 (m, 2H), 2.78 (s, 6H).

### Example 46C

Preparation of 2-(hydroxy(2,6,6-trimethyl-4-oxo-4,5,6,7-tetrahydro-1H-indol-3-yl)methyl)-N,N-dimethylbenzenesulfonamide. To a solution of 2,6,6-trimethyl-6,7-dihydro-1H-indol-4(5H)-one (0.300 g, 1.69 mmol) and 2-formyl-N,N-dimethylbenzenesulfonamide (0.361 g, 1.69 mmol) in MeOH (8 mL) was added an aqueous solution of 3M sodium hydroxide (1.69 mL, 5.08 mmol). The reaction was heated in a sealed tube at 60° C. for 16 hours, after which the reaction was diluted in water, leading to the formation of a precipitate which was filtered and collected. Purification of this solid by silica gel chromatography (Luknova 40 g, 20 mL/min) using 1 to 7% of 7:1 MeCN:MeOH in dichloromethane over 70 minutes afforded 2-(hydroxy(2,6,6-trimethyl-4-oxo-4,5,6,7-tetrahydro-1H-indol-3-yl)methyl)-N,N-dimethylbenzenesulfonamide (0.179 g, 0.458 mmol, 27% yield) as a solid.

### Example 46D

Preparation of N,N-dimethyl-2-((2,6,6-trimethyl-4-oxo-4,5,6,7-tetrahydro-1H-indol-3-yl)methyl)benzenesulfonamide. To a 0° C. solution of 2-(hydroxy(2,6,6-trimethyl-4-oxo-4,5,6,7-tetrahydro-1H-indol-3-yl)methyl)-N,N-dimethylbenzenesulfonamide (0.179 g, 0.458 mmol) in dichloromethane (5 mL) was added trimethylsilyl trifluoromethanesulfonate (0.083 mL, 0.458 mmol) leading to the formation of a bright orange color. After 5 minutes at 0° C., triethylsilane was added (0.080 mL, 0.504 mmol). After 20 minutes, the reaction was complete. The reaction was quenched by the addition of saturated sodium bicarbonate solution, extracted with dichloromethane, dried (sodium sulfate), filtered and concentrated to afford crude N,N-dimethyl-2-((2,6,6-trimethyl-4-oxo-4,5,6,7-tetrahydro-1H-indol-3-yl)methyl)benzenesulfonamide (105 mg, 0.280 mmol, 61% yield), which was used in the next step without further purification.). ¹H NMR (400 MHz, CD₃OD) δ (ppm): 7.89 (dd, 1H, J=8.0 Hz, J=1.6 Hz), 7.39 (ddd, 1H, J=7.6 Hz, J=7.2 Hz, J=1.6 Hz), 7.31 (m, 1H), 6.99 (dd, 1H, J=8.0 Hz, J=0.8 Hz), 4.82 (s, 2H), 4.41 (s, 2H), 2.87 (s, 6H), 2.69 (s, 2H), 2.25 (s, 2H), 2.01 (s, 3H), 1.12 (s, 6H).

### Example 46E

Preparation of ethyl 2-(3-(2-(N,N-dimethylsulfamoyl)benzyl)-2,6,6-trimethyl-4-oxo-4,5,6,7-tetrahydro-1H-indol-1-yl)acetate (I-68). To a sealed tube containing a solution N,N-dimethyl-2-((2,6,6-trimethyl-4-oxo-4,5,6,7-tetrahydro-1H-indol-3-yl)methyl)benzenesulfonamide (0.105 g, 0.280 mmol) in acetonitrile (12 mL) was added ethyl 2-bromoacetate (0.0470 mL, 0.421 mmol), potassium iodide (0.0930 g, 0.561 mmol), and potassium carbonate (0.077 g, 0.561 mmol). The tube was sealed and heated to 85° C. for 16 hours, after which the reaction was complete. The mixture was cooled to room temperature then quenched by the addition of saturated ammonium chloride solution, extracted with dichloromethane, dried (sodium sulfate), filtered and concentrated to a residue. Purification by silica gel chromatography (Luknova 40 g, 20 mL/min) using 1 to 7% of 7:1 MeCN:MeOH in dichloromethane over 70 minutes afforded ethyl 2-(3-(2-(N,N-dimethylsulfamoyl)benzyl)-2,6,6-trimethyl-4-oxo-4,5,6,7-tetrahydro-1H-indol-1-yl)acetate (0.0460 g, 0.100 mmol, 36% yield).

### Example 46F

Preparation of 2-(3-(2-(N,N-dimethylsulfamoyl)benzyl)-2,6,6-trimethyl-4-oxo-4,5,6,7-tetrahydro-1H-indol-1-yl)acetic acid (I-52). To a solution of ethyl 2-(3-(2-(N,N-dimethylsulfamoyl)benzyl)-2,6,6-trimethyl-4-oxo-4,5,6,7-tetrahydro-1H-indol-1-yl)acetate (0.0460 g, 0.100 mmol) in THF (3 mL) and water (3 mL) was added an aqueous 3M solution of sodium hydroxide (0.0670 mL, 0.200 mmol). The reaction was stirred at room temperature for 20 minutes, after which it was concentrated to remove the THF, then neutralized by the addition of 3M aqueous hydrochloric acid (0.0670 mL). The resulting precipitate was filtered and dried to afford 2-(3-(2-(N,N-dimethylsulfamoyl)benzyl)-2,6,6-trimethyl-4-oxo-4,5,6,7-tetrahydro-1H-indol-1-yl)acetic acid (10.5 mg, 0.0240 mmol, 24% yield). ¹H NMR (400 MHz, CD₃CO) δ (ppm): 7.89 (dd, 1H, J=7.6 Hz, J=1.2 Hz), 7.38 (ddd, 1H, J=7.6 Hz, J=7.2 Hz, J=1.2 Hz), 7.30 (m, 1H, 7.05 (d, 1H, J=8.0 Hz), 4.54 (s, 2H), 4.45 (s, 2H), 2.87 (s, 6H), 2.68 (s, 2H), 2.26 (s, 2H), 2.00 (s, 3H), 1.13 (s, 6H).

### Example 47

### Preparation of ethyl 2-(2,6,6-trimethyl-3-(2-(N-methyl-N-phenylsulfamoyl)benzyl)-4-oxo-4,5,6,7-tetrahydro-1H-indol-1-yl)acetate (I-66) and 2-(2,6,6-trimethyl-3-(2-(N-methyl-N-phenylsulfamoyl)benzyl)-4-oxo-4,5,6,7-tetrahydro-1H-indol-1-yl)acetic acid (I-49)

### Example 47A

Preparation of (2-formylbenzene-1-sulfonyl chloride) Sulfurous dichloride (33.7 g, 284 mmol) was added to sodium 2-formylbenzenesulfonate (7.20 g, 34.6 mmol) was added followed by a catalytic amount of DMF (150 μL). The reaction was stirred for two minutes, after which an exotherm was observed. The reaction was then refluxed at 100° C. for three minutes. Water was then added slowly to the resulting yellow solution, and the mixture was extracted with pentane, dried (sodium sulfate), filtered and concentrated to afford 2-formylbenzene-1-sulfonyl chloride as clear residue. This material was used in the next step without further purification.

### Example 47B

Preparation of 2-formyl-N-methyl-N-phenylbenzenesulfonamide. To a solution of crude 2-formylbenzene-1-sulfonyl chloride (0.500 g, 2.44 mmol) in dichloromethane (12 mL) was added N-methylaniline (0.266 ml, 2.44 mmol). The reaction stirred at room temperature for 3 hours, after which the solvent was removed in vacuo. The resulting residue was diluted in diethyl ether, after which an aqueous solution of sodium bisulfite (0.454 g, 4.36 mmol) was added. The reaction was stirred at room temperature for 16 hours. The layers were separated, and the aqueous layer was washed with diethyl ether, then treated with sodium carbonate (0.462 g, 4.36 mmol) and THF. The resulting solution was stirred at room temperature for 4 hours then heated to 40° C. The reaction was then cooled, extracted with dichloromethane, dried (sodium sulfate), filtered and concentrated to afford 2-formyl-N-methyl-N-phenylbenzenesulfonamide (0.151 g, 0.548 mmol, 25% yield).

### Example 47C

Preparation of 2-(hydroxy(2,6,6-trimethyl-4-oxo-4,5,6,7-tetrahydro-1H-indol-3-yl)methyl)-N-methyl-N-phenylbenzenesulfonamide. To a solution of 2,6,6-trimethyl-6,7-dihydro-1H-indol-4(5H)-one (0.515 g, 2.91 mmol) and 2-formyl-N-methyl-N-phenylbenzenesulfonamide (0.800 g, 2.91 mmol) in MeOH (5 mL) was added an aqueous 3M solution of sodium hydroxide (2.91 mL, 8.72 mmol). The reaction was heated to 65° C. in a sealed tube for 16 hours, then cooled to room temperature, and diluted with water, leading to the formation of an off-white precipitate which was purified by silica gel chromatography using a gradient of 1 to 5% MeOH in dichloromethane. The product, 2-(hydroxy(2,6,6-trimethyl-4-oxo-4,5,6,7-tetrahydro-1H-indol-3-yl)methyl)-N-methyl-N-phenylbenzenesulfonamide (0.265 g, 0.586 mmol, 20% yield) was isolated as an off-white solid.

### Example 47D

Preparation of N-methyl-N-phenyl-2-((2,6,6-trimethyl-4-oxo-4,5,6,7-tetrahydro-1H-indol-3-yl)methyl)benzenesulfonamide. To a 0° C. solution of 2-(hydroxy(2,6,6-trimethyl-4-oxo-4,5,6,7-tetrahydro-1H-indol-3-yl)methyl)-N-methyl-N-phenylbenzenesulfonamide (0.265 g, 0.586 mmol) in dichloromethane (10 ml) was added trimethylsilyl trifluoromethanesulfonate (0.106 ml, 0.586 mmol), leading to the formation of a bright orange reaction color. After five minutes at 0° C., triethylsilane (0.103 ml, 0.644 mmol) was added. After 20 minutes, the reaction was still incomplete so additional trimethylsilyl trifluoromethanesulfonate (1.00 equiv.) and triethylsilane (1.00 equiv.) were added. The reaction was then quenched by the addition of saturated sodium bicarbonate solution, extracted with dichloromethane, dried (sodium sulfate), filtered and concentrated. Purification by silica gel chromatography using 1 to 7% of 7:1 MeCN:MeOH in dichloromethane over 70 minutes afforded N-methyl-N-phenyl-2-((2,6,6-trimethyl-4-oxo-4,5,6,7-tetrahydro-1H-indol-3-yl)methyl)benzenesulfonamide (0.0804 g, 0.184 mmol, 28% yield) as an off-white solid.

### Example 47E

Preparation of ethyl 2-(2,6,6-trimethyl-3-(2-(N-methyl-N-phenylsulfamoyl)benzyl)-4-oxo-4,5,6,7-tetrahydro-1H-indol-1-yl)acetate (I-66). To a solution of N-methyl-N-phenyl-2-((2,6,6-trimethyl-4-oxo-4,5,6,7-tetrahydro-1H-indol-3-yl)methyl)benzenesulfonamide (0.0804 g, 0.184 mmol) in acetonitrile (7.4 mL) in a sealed tube was added ethyl 2-bromoacetate (0.0310 mL, 0.276 mmol), potassium carbonate (0.0510 g, 0.368 mmol), and potassium iodide (0.0610 g, 0.368 mmol). The reaction was sealed and heated to 85° C. for 16 hours, after which the reaction cooled to room temperature, treated with saturated ammonium chloride solution, extracted with dichloromethane (3×30 mL), dried (sodium sulfate), filtered and concentrated to a residue which was purified by semi-preparative reverse phase HPLC using 10 to 90% acetonitrile (spiked with 0.1% trifluoroacetic acid) in water (spiked with 0.1% trifluoroacetic acid) affording ethyl 2-(2,6,6-trimethyl-3-(2-(N-methyl-N-phenylsulfamoyl)benzyl)-4-oxo-4,5,6,7-tetrahydro-1H-indol-1-yl)acetate (0.0166 g, 0.0320 mmol, 17% yield). ¹H NMR (400 MHz, CD₃OD) δ (ppm): 7.70 (d, 1H, J=8.0 Hz), 7.19-7.36 (m, 7H), 6.94 (d, 1H, J=7.6 Hz), 4.76 (s, 2H), 4.35 (s, 2H), 4.23 (q, 2H, J=7.2 Hz), 3.32 (s, 3H), 2.66 (s, 2H), 2.28 (s, 2H), 1.92 (s, 3H), 1.29 (t, 3H, J=6.8), 1.12 (s, 6H).

### Example 47F

Preparation of 2-(2,6,6-trimethyl-3-(2-(N-methyl-N-phenylsulfamoyl)benzyl)-4-oxo-4,5,6,7-tetrahydro-1H-indol-1-yl)acetic acid (I-49). To a solution of ethyl 2-(2,6,6-trimethyl-3-(2-(N-methyl-N-phenylsulfamoyl)benzyl)-4-oxo-4,5,6,7-tetrahydro-1H-indol-1-yl)acetate (0.0166 g, 0.0320 mmol) in water (3.0 ml) and THF (3.0 ml) was added an aqueous 3M solution of sodium hydroxide (0.0210 ml, 0.0640 mmol). The reaction was stirred for one hour at room temperature, after which the THF was removed in vacuo, then neutralized with aqueous 3M hydrochloric acid (0.0210 mL) leading to the formation of a precipitate which was further purified by semi-preparative reverse phase HPLC using 10 to 90% acetonitrile (spiked with 0.1% trifluoroacetic acid) in water (spiked with 0.1% trifluoroacetic acid). The product 2-(2,6,6-trimethyl-3-(2-(N-methyl-N-phenylsulfamoyl)benzyl)-4-oxo-4,5,6,7-tetrahydro-1H-indol-1-yl)acetic acid (5.3 mg, 11 μmmol, 34% yield) was obtained as an off-white solid. ¹H NMR (400 MHz, CD₃OD) δ (ppm): 7.70 (dd, 1H, J=8.0 Hz, J=1.2 Hz), 7.19-7.37 (m, 7H), 6.96 (d, 1H, J=7.6 Hz), 4.71 (s, 2H), 4.34 (s, 2H), 3.33 (s, 3H), 2.66 (s, 2H), 2.27 (s, 2H), 1.94 (s, 3H), 1.12 (s, 6H).

### Example 48

### Preparation of ethyl 2-(2,6,6-trimethyl-3-(4-(morpholinosulfonyl)benzyl)-4-oxo-4,5,6,7-tetrahydro-1H-indol-1-yl)acetate (I-67) and 2-(2,6,6-trimethyl-3-(4-(morpholinosulfonyl)benzyl)-4-oxo-4,5,6,7-tetrahydro-1H-indol-1-yl)acetic acid (I-45)

### Example 48A

Preparation of (4-(chlorosulfonyl)phenyl)methylene diacetate. To a solution of .p-toluenesulfonyl chloride (10.0 g, 52.7 mmol) at 0° C. in acetic anhydride (100 mL), and acetic acid (100 mL), was added concentrated sulfuric acid (16.0 mL). Upon completion of addition, solid chromium trioxide (8.61 g, 84.4 mmol) was added portionwise (6 additions over 15 minutes). The reaction was stirred at 0° C. for 30 minutes, after which it was cautiously quenched with ice (extremely exothermic). An additional 100 mL ice water was added to reaction, leading to the formation of a precipitate which was filtered and collected. To this precipitate was added a solution of saturated sodium bicarbonate, and the resulting solution was stirred at room temperature for 2.5 hours, then extracted with ethyl acetate, dried (sodium sulfate), filtered and concentrated to an off-white solid which was shown to be 80% pure (contaminated with p-toluenesulfonyl chloride). This crude mixture (4-(chlorosulfonyl)phenyl)methylene diacetate (4.34 g, 21% yield based on ¹H-NMR) was taken on to the next step without further purification.

### Example 48B

Preparation of (4-(morpholinosulfonyl)phenyl)methylene diacetate. To a solution of crude (4-(chlorosulfonyl)phenyl)methylene diacetate (1.00 g, 3.26 mmol) in THF (3.6 mL) at room temperature was added morpholine (0.625 mL, 7.17 mmol). The reaction was complete after 30 minutes, after which a white precipitate was observed, this said, was filtered and dried to afford a crude mixture of (4-(morpholinosulfonyl)phenyl)methylene diacetate (1.17 g) which was used in the next step without further purification.

### Example 48C

Preparation of 4-(morpholinosulfonyl)benzaldehyde. To a solution of (4-(morpholinosulfonyl)phenyl)methylene diacetate (1.17 g, 3.26 mmol) in MeOH (20 mL) was added potassium carbonate (0.667 g, 4.82 mmol) and reaction was stirred at room temperature for 16 hours. The solvent was then removed, The resulting mixture was extracted with dichloromethane, dried (sodium sulfate), filtered and concentrated to a residue which was purified by silica column chromatography using 50% ethyl acetate in hexane. The product 4-(morpholinosulfonyl)benzaldehyde (0.395 g, 1.55 mmol, 48% yield) was isolated as a white solid. ¹H NMR (400 MHz, CDCl₃) δ (ppm): 10.13 (s, 1H), 8.08 (dd, 2H, J=6.4 Hz, J=1.6 Hz), 7.93 (dd, 2H, J=6.4 Hz, J=1.6 Hz), 3.77-3.73 (m, 4H), 3.06-3.04 (m, 4H).

### Example 48D

Preparation of 3-(hydroxy(4-(morpholinosulfonyl)phenyl)methyl)-2,6,6-trimethyl-6,7-dihydro-1H-indol-4(5H)-one. To a solution of 2,6,6-trimethyl-6,7-dihydro-1H-indol-4(5H)-one (0.153 g, 0.862 mmol) and 4-(morpholinosulfonyl)benzaldehyde (0.220 g, 0.862 mmol) in MeOH (4.3 mL) in a sealed tube was added a 1M solution of aqueous sodium hydroxide (2.59 mL, 2.59 mmol). The vial was sealed and heated to 60° C. for 16 hours, after which the reaction was cooled to room temperature and diluted with water. A light-tan precipitate formed which was collected and dried to afford 3-(hydroxy(4-(morpholinosulfonyl)phenyl)methyl)-2,6,6-trimethyl-6,7-dihydro-1H-indol-4(5H)-one (0.200 g, 0.462 mmol, 54% yield). ¹H NMR (400 MHz, CDCl₃) δ (ppm): 8.01 (br s, 1H), 7.64 (d, 2H, J=8.4 Hz), 7.25 (d, 2H, J=8.0 Hz), 5.71 (s, 1H), 3.71-3.74 (m, 4H), 2.95-2.98 (m, 4H), 2.66 (d, 1H, J=16.0 Hz), 2.59 (d, 1H, J=16.4 Hz), 2.40 (d, 1H, J=16.4 Hz), 2.31 (d, 1H, J=16.4 Hz), 2.26 (s, 3H), 1.10 (s, 6H, two shifts isochronous).

### Example 48E

Preparation of 2,6,6-trimethyl-3-(4-(morpholinosulfonyl)benzyl)-6,7-dihydro-1H-indol-4(5H)-one. To a 0° C. solution of 3-(hydroxy(4-(morpholinosulfonyl)phenyl)methyl)-2,6,6-trimethyl-6,7-dihydro-1H-indol-4(5H)-one (0.200 g, 0.462 mmol) in dichloromethane (5.1 mL) was added trimethylsilyl trifluoromethanesulfonate (0.0840 mL, 0.462 mmol) leading to the formation of a bright orange reaction color. After 5 minutes at 0° C., triethylsilane was added (0.0810 mL, 0.509 mmol). After 20 minutes, the reaction was complete, then was quenched by the addition of saturated sodium bicarbonate solution, extracted with dichloromethane, dried (sodium sulfate), filtered and concentrated to afford crude 2,6,6-trimethyl-3-(4-(morpholinosulfonyl)benzyl)-6,7-dihydro-1H-indol-4(5H)-one which was used in the next step without further purification. ¹H NMR (400 MHz, CDCl₃) δ (ppm): 7.80 (br s, 1H), 7.59 (dd, 1H, J=8.4 Hz, J=2.0 Hz), 7.42 (d, 2H, J=8.4 Hz), 4.13 (s, 2H), 3.71-3.73 (m, 4H). 2.95-2.97 (m, 4H), 2.61 (s, 2H), 2.31 (s, 2H), 2.16 (s, 3H), 1.10 (s, 6H).

### Example 48F

Preparation of ethyl 2-(2,6,6-trimethyl-3-(4-(morpholinosulfonyl)benzyl)-4-oxo-4,5,6,7-tetrahydro-1H-indol-1-yl)acetate (I-67). To a sealed tube containing a solution of 2,6,6-trimethyl-3-(4-(morpholinosulfonyl)benzyl)-6,7-dihydro-1H-indol-4(5H)-one (0.200 g, 0.480 mmol) in acetonitrile (12.0 mL) was added ethyl 2-bromoacetate (0.0800 mL, 0.720 mmol), potassium iodide (0.016 g, 0.096 mmol), and potassium carbonate (0.133 g, 0.960 mmol). The tube was sealed and heated to 85° C. for 16 hours, after which the reaction was quenched by the addition of saturated ammonium chloride solution, extracted with dichloromethane, dried (sodium sulfate), filtered and concentrated to a residue. Purification by silica gel chromatography (Luknova 40 g, 20 mL/min) using 1 to 7% of 7:1 MeCN:MeOH in dichloromethane over 70 minutes afforded ethyl 2-(2,6,6-trimethyl-3-(4-(morpholinosulfonyl)benzyl)-4-oxo-4,5,6,7-tetrahydro-1H-indol-1-yl)acetate (0.147 g, 0.292 mmol, 61% yield). ¹H NMR (400 MHz, CDCl₃) δ (ppm): 7.58 (d, 2H, J=8.4 Hz), 7.39 (d, 2H, J=8.4 Hz), 4.51 (s, 2H), 4.25 (q, 2H, J=7.2 Hz), 4.18 (s, 2H), 3.71-3.73 (m, 4H), 2.95-2.97 (m, 4H), 2.53 (s, 2H), 2.32 (s, 2H), 2.09 (s, 3H), 1.29 (t, 3H, J=7.2 Hz), 1.11 (s, 6H).

### Example 48G

Preparation of 2-(2,6,6-trimethyl-3-(4-(morpholinosulfonyl)benzyl)-4-oxo-4,5,6,7-tetrahydro-1H-indol-1-yl)acetic acid (I-45). To a solution of ethyl 2-(2,6,6-trimethyl-3-(4-(morpholinosulfonyl)benzyl)-4-oxo-4,5,6,7-tetrahydro-1H-indol-1-yl)acetate (0.147 g, 0.292 mmol) in THF (0.7 mL) and water (0.5 mL) was added a 1M aqueous solution of sodium hydroxide (0.585 mL, 0.585 mmol). The reaction was stirred at room temperature for one hour, then quenched by the addition of 1M aqueous hydrochloric acid solution (0.585 mL), extracted with dichloromethane, dried (sodium sulfate), filtered and concentrated to a crude solid which was purified by semi-preparative reverse phase HPLC using 10 to 90% acetonitrile (spiked with 0.1% trifluoroacetic acid) in water (spiked with 0.1% trifluoroacetic acid). The product 2-(2,6,6-trimethyl-3-(4-(morpholinosulfonyl)benzyl)-4-oxo-4,5,6,7-tetrahydro-1H-indol-1-yl)acetic acid (0.060 g, 0.126 mmol, 43% yield) was isolated as a white solid. ¹H NMR (400 MHz, CDCl₃) δ (ppm): 7.58 (d, 2H, J=8.4 Hz), 7.39 (d, 2H, J=8.4 Hz), 4.59 (s, 2H), 4.18 (s, 2H), 3.71-3.73 (m, 4H), 2.94-2.96 (m, 4H), 2.55 (s, 2H), 2.33 (s, 2H), 2.12 (s, 3H), 1.11 (s, 6H).

### Example 49

### Preparation of ethyl 2-(2,6,6-trimethyl-4-oxo-3-(4-(pyrrolidin-1-ylsulfonyl)benzyl)-4,5,6,7-tetrahydro-1H-indol-1-yl)acetate (I-65) and 2-(2,6,6-trimethyl-4-oxo-3-(4-(pyrrolidin-1-ylsulfonyl)benzyl)-4,5,6,7-tetrahydro-1H-indol-1-yl)acetic acid (I-44)

### Example 49A

Preparation of 3-(hydroxy(4-(pyrrolidin-1-ylsulfonyl)phenyl)methyl)-2,6,6-trimethyl-6,7-dihydro-1H-indol-4(5H)-one. To a solution of 2,6,6-trimethyl-6,7-dihydro-1H-indol-4(5H)-one (152 mg, 0.860 mmol) in methanol (1.7 mL) at 0° C. was added solid 4-(pyrrolidin-1-ylsulfonyl)benzaldehyde, which was prepared via the same method as 4-(morpholinosulfonyl)benzaldehyde (226 mg, 0.946 mmol). At 0° C., a 1M aqueous solution of sodium hydroxide (2.58 mL, 2.58 mmol) was added. The reaction was stirred at 0° C. for 30 minutes, after the reaction was heated to 60° C. for 12 hours after which the reaction was diluted in water, extracted with dichloromethane (3×40 mL), dried (sodium sulfate), filtered and concentrated to a residue which was purified on silica gel (Luknova 25 g, 20 mL/min) using 1 to 10% 7:1 MeCN:MeOH in dichloromethane over 100 minutes affording 3-(hydroxy(4-(pyrrolidin-1-ylsulfonyl)phenyl)methyl)-2,6,6-trimethyl-6,7-dihydro-1H-indol-4(5H)-one (51.7 mg, 0.124 mmol, 14% yield) as a white solid. ¹H NMR (400 MHz, CDCl₃) δ (ppm): 8.78 (br s, 1H), 7.70 (dd, 2H, J=6.8 Hz, J=2.0 Hz), 7.48 (d, 2H, J=8.8 Hz), 7.06 (d, 1H, J=11.2 Hz), 5.68 (d, 1H, J=11.2 Hz), 3.19-3.23 (m, 4H), 2.60 (d, 1H, J=16.4 Hz), 2.55 (d, 1H, J=16.0 Hz), 2.35 (d, 1H, J=16.4 Hz), 2.28 (d, 1H, J=16.4 Hz), 1.74 (m, 4H), 1.06 (s, 3H), 1.05 (s, 3H).

### Example 49B

Preparation of 2,6,6-trimethyl-3-(4-(pyrrolidin-1-ylsulfonyl)benzyl)-6,7-dihydro-1H-indol-4(5H)-one. To a 0° C. solution of 3-(hydroxy(4-(pyrrolidin-1-ylsulfonyl)phenyl)methyl)-2,6,6-trimethyl-6,7-dihydro-1H-indol-4(5H)-one (50.0 mg, 0.120 mmol) in dichloromethane (3 mL) was added trimethylsilyl trifluoromethanesulfonate (0.0220 mL, 0.120 mmol). The reaction turned bright gold. After 3 minutes, triethylsilane (0.0210 mL, 0.132 mmol) was added, and the gold color was discharged. After 20 minutes, the reaction was diluted in saturated sodium bicarbonate solution, extracted with dichloromethane (3×30 mL), dried (sodium sulfate), filtered, and concentrated to a white solid 2,6,6-trimethyl-3-(4-(pyrrolidin-1-ylsulfonyl)benzyl)-6,7-dihydro-1H-indol-4(5H)-one. Used without further purification. ¹H NMR (400 MHz, CDCl₃) δ (ppm): 7.79 (br s, 1H), 7.67 (dd, 2H, J=6.8 Hz, J=2.0 Hz), 7.37 (d, 2H, J=8.4 Hz), 4.14 (s, 2H), 3.20-3.23 (m, 4H), 2.62 (s, 2H), 2.31 (s, 2H), 2.14 (s, 3H), 1.72-1.76 (m, 4H), 1.11 (s, 6H).

### Example 49C

Preparation of ethyl 2-(2,6,6-trimethyl-4-oxo-3-(4-(pyrrolidin-1-ylsulfonyl)benzyl)-4,5,6,7-tetrahydro-1H-indol-1-yl)acetate (I-65). To a solution of 2,6,6-trimethyl-3-(4-(pyrrolidin-1-ylsulfonyl)benzyl)-6,7-dihydro-1H-indol-4(5H)-one (56.4 mg, 0.141 mmol) in acetonitrile (3.5 mL) in a sealed tube was added ethyl bromoacetate (31.4 μL, 0.282 mmol), potassium carbonate (29.2 mg, 0.211 mmol), and potassium iodide (4.7 mg, 0.028 mmol). The reaction was sealed and heated to 85° C. After 16 hours, the was diluted with saturated ammonium chloride solution and extracted with dichloromethane (3×30 mL), dried (sodium sulfate), filtered, and concentrated to a yellow residue. Purification by column chromatography on silica gel (Luknova 25 g, 20 mL/min) using 1 to 7% of 7:1 MeCN:MeOH in dichloromethane over 70 minutes furnished ethyl 2-(2,6,6-trimethyl-4-oxo-3-(4-(pyrrolidin-1-ylsulfonyl)benzyl)-4,5,6,7-tetrahydro-1H-indol-1-yl)acetate (31.8 mg, 0.0650 mmol, 46.4% yield). ¹H NMR (400 MHz, CDCl₃) δ (ppm): 7.65 (d, 2H, J=8.4 Hz), 7.34 (d, 2H, J=8.4 Hz), 4.50 (s, 2H), 4.23 (q, 2H, J=7.2 Hz), 4.17 (s, 2H), 3.18-3.21 (m, 4H), 2.52 (s, 2H), 2.30 (s, 2H), 2.06 (s, 3H), 1.71-1.74 (m, 4H), 1.28 (t, 3H, J=7.2 Hz), 1.10 (s, 6H).

### Example 49D

Preparation of 2-(2,6,6-trimethyl-4-oxo-3-(4-(pyrrolidin-1-ylsulfonyl)benzyl)-4,5,6,7-tetrahydro-1H-indol-1-yl)acetic acid (I-44). To a solution of ethyl 2-(2,6,6-trimethyl-4-oxo-3-(4-(pyrrolidin-1-ylsulfonyl)benzyl)-4,5,6,7-tetrahydro-1H-indol-1-yl)acetate (31.8 mg, 0.0650 mmol) in THF (653 μL) and water (653 μL) was added an aqueous 1M solution of sodium hydroxide (131 μL, 0.131 mmol). The reaction was stirred at room temperature for 30 minutes after which the reaction was quenched by the addition of 3M hydrochloric acid (0.043 mL), extracted with dichloromethane (3×30 mL), dried (sodium sulfate), filtered, and concentrated to an off-white solid 2-(2,6,6-trimethyl-4-oxo-3-(4-(pyrrolidin-1-ylsulfonyl)benzyl)-4,5,6,7-tetrahydro-1H-indol-1-yl)acetic acid (25.1 mg, 0.0550 mmol, 84% yield). ¹H NMR (400 MHz, CDCl₃) δ (ppm): 7.63 (d, 2H, J=8.4 Hz), 7.33 (d, 2H, J=8.4 Hz), 4.47 (s, 2H), 4.15 (s, 2H), 3.17-3.21 (m, 4H), 2.49 (s, 2H), 2.27 (s, 2H), 2.04 (s, 3H), 1.71-1.75 (m, 4H), 1.10 (s, 6H).

### Example 50

### Preparation of 2-(5,5-difluoro-2-methyl-3-(2-(morpholinosulfonyl)benzyl)-4,5,6,7-tetrahydro-1H-indol-1-yl)acetic acid (I-39)

Synthesis was carried out as for compound I-17 except 4,4-difluorocyclohexanone was used as the starting material instead of 4,4-dimethylcyclohexanone. ¹H-NMR (400 MHz d-MeOH) δ ppm: 7.92 (d, J=8.0 Hz, 1H), 7.40 (m, 2H), 7.20 (d, J=7.8 Hz, 1H), 4.58 (s, 2H), 4.11 (s, 2H), 3.70 (m, 4H), 3.17 (m, 4H), 2.68 (m, 4H), 2.17 (m, 2H), 2.04 (s, 3H), 2.03 (s, 3H).

General Synthetic Scheme for Preparation of Esters.

In Scheme 11 and Example 51-54, esters generally may be prepared as described for compound I-58, wherein R is a cyclic or non-cyclic substituent or wherein two R may be joined to form a heterocycle or heteroaryl group. R′ is a lower alkyl; in particular, it may be methyl, ester, propyl, isopropyl, n-butyl, t-butyl or s-butyl.

### Example 51

Preparation of Methyl 2-(2,6,6-trimethyl-4-oxo-3-(2-(pyrrolidin-1-ylsulfonyl)benzyl)-4,5,6,7-tetrahydro-1H-indol-1-yl)acetate (I-58). ¹H-NMR (400 MHz CDCl₃): δ 7.98 (d, J=8 Hz, 1H), 7.31 (t, J=8 Hz, 1H), 7.22 (t, J=8 Hz, 1H), 6.93 (d, J=8 Hz, 1H), 4.56 (s, 2H), 4.54 (s, 2H), 3.80 (s, 3H), 3.3-3.4 (m, 4H), 2.56 (s, 2H), 2.29 (s, 2H), 1.98 (s, 3H), 1.9-2.0 (m, 4H), 1.12 (s, 6H) ppm.

### Example 52

Preparation of isopropyl 2-(2,6,6-trimethyl-4-oxo-3-(2-(pyrrolidin-1-ylsulfonyl)benzyl)-4,5,6,7-tetrahydro-1H-indol-1-yl)acetate (I-55). ¹H-NMR (CDCl₃): δ 7.99 (d, J=6 Hz, 1H), 7.30 (t, J=6 Hz, 1H), 7.22 (t, J=6 Hz, 1H), 6.93 (d, J=6 Hz, 1H), 5.09 (hep, J=6 Hz, 1H), 4.54 (s, 2H), 4.50 (s, 2H), 3.80 (s, 3H), 3.35-3.45 (m, 4H), 2.56 (s, 2H), 2.29 (s, 2H), 1.98 (s, 3H), 1.9-2.0 (m, 4H), 1.25 (d, J=6 Hz), 1.13 (s, 6H) ppm.

### Example 53

Preparation of methyl 2-(2,6,6-trimethyl-3-(2-(morpholinosulfonyl)benzyl)-4-oxo-4,5,6,7-tetrahydro-1H-indol-1-yl)acetate (I-59). ¹H-NMR (400 MHz CDCl₃): δ 7.96 (dd, J=8.1 Hz, 1H), 7.35 (td, J=8.1 Hz, 1H), 7.26 (td, J=8.1 Hz, 1H), 6.95 (td, J=8.1 Hz, 1H), 4.51 (s, 2H), 4.50 (s, 2H), 3.81 (s, 3H), 3.75-3.85 (m, 4H), 3.25-3.35 (m, 4H), 2.55 (s, 2H), 2.28 (s, 2H), 1.99 (s, 3H), 1.13 (s, 6H) ppm.

### Example 54

Preparation of isopropyl 2-(2,6,6-trimethyl-3-(2-(morpholinosulfonyl)benzyl)-4-oxo-4,5,6,7-tetrahydro-1H-indol-1-yl)acetate (I-60). ¹H-NMR (400 MHz CDCl₃): δ 7.96 (dd, J=8.1 Hz, 1H), 7.34 (td, J=8.1 Hz, 1H), 7.25 (td, J=8.1 Hz, 1H), 6.95 (td, J=8.1 Hz, 1H), 5.10 (sep, J=6 Hz, 1H), 4.54 (s, 2H), 4.51 (s, 2H), 3.75-3.85 (m, 4H), 3.25-3.35 (m, 4H), 2.57 (s, 2H), 2.29 (s, 2H), 1.99 (s, 3H), 1.27 (d, J=6 Hz, 6H), 1.13 (s, 6H) ppm.

### Example 55

Preparation of 2-morpholinoethyl 2-(2,6,6-trimethyl-4-oxo-3-(2-(pyrrolidin-1-ylsulfonyl)benzyl)-4,5,6,7-tetrahydro-1H-indol-1-yl)acetate (I-62). Ethyl 2-(2,6,6-trimethyl-4-oxo-3-(2-(pyrrolidin-1-ylsulfonyl)benzyl)-4,5,6,7-tetrahydro-1H-indol-1-yl)acetate (0.242 g, 0.497 mmol) was dissolved in 2-morpholinoethanol (2.72 mL, 0.497 mmol), to this was added (Ti(OCH(CH₃)₂)₄) (0.016 mL, 0.055 mmol) and the reaction was heated to 90° C. LCMS after 2 hours shows a large amount of starting material and a small amount of product. The reaction was cooled back to RT and stirred for 48 h. LCMS showed consumption of all SM. The reaction was concentrated in vacuo and the oil was dissolved in diethyl ether. The organics were washed with water (3×50 mL) and the dried with magnesium sulfate, concentrated, and purified via silica gel chromatography. The oil was then taken up the water (20 mL) and allowed to precipitate overnight. The resulting white powder was filtered to afford 2-morpholinoethyl 2-(2,6,6-trimethyl-4-oxo-3-(2-(pyrrolidin-1-ylsulfonyl)benzyl)-4,5,6,7-tetrahydro-1H-indol-1-yl)acetate (120 mg, 0.210 mmol, 42.2 yield) as a fine white powder. ¹H-NMR (400 MHz CDCl₃): δ 7.92 (d, J=8.8 Hz, 1H), 7.20 (m, 2H), 6.90 (d, J=8.7 Hz, 1H), 4.48 (d, J=7.2 Hz, 4H), 4.25 (t, J=5.6, 11.2 Hz 2H), 3.59 (t, J=4.4, 9.2 Hz 4H), 3.32 (t, J=6.8, 13.6 Hz 4H), 2.54 (m, 4H), 2.38 (m, 4H), 2.23 (s, 2H), 1.93 (s, 3H), 1.87 (m, 4H), 1.06 (s, 6H) ppm.

Compounds in which L is —S(O)m-, wherein m is 0, 1 or 2, may be synthesized by the following route:

### Scheme 12: General synthetic scheme for compounds in which L is —S(O)n-

### Example 56

### Preparation of ethyl 2-(2,5,5-trimethyl-3-(4-(morpholinosulfonyl)phenylthio)-4,5,6,7-tetrahydro-1H-indol-1-yl)acetate (I-71) and 2-(2,5,5-trimethyl-3-(4-(morpholinosulfonyl)phenylthio)-4,5,6,7-tetrahydro-1H-indol-1-yl)acetic acid (I-37)

### Example 56A

### Preparation of 2-(2,5,5-trimethyl-3-(phenylthio)-4,5,6,7-tetrahydro-1H-indol-1-yl)acetic acid

To a solution of ethyl 2-(2,5,5-trimethyl-4,5,6,7-tetrahydro-1H-indol-1-yl)acetate (0.2 g, 0.802 mmol) and benzenethiol (0.082 ml, 0.802 mmol) stirring in DMF (5 ml) was added iodine (0.204 g, 0.802 mmol). The reaction was stirred for 12 hours at which time sodium hydroxide (3.21 ml, 3.21 mmol) was added. The reaction was stirred another 12 hours. The solution was adjusted to a pH of 3 using 3N HCl and stirred for 10 minutes. The reaction mixture was concentrated in vacuo and reconstituted in ethyl acetate which was washed with brine. The organic layer was dried over sodium sulfate, concentrated, reconstituted in acetonitrile and purified by reverse phase HPLC using water and acetonitrile (both spiked with 0.1% TFA) to afford the desired product 2-(2,5,5-trimethyl-3-(phenylthio)-4,5,6,7-tetrahydro-1H-indol-1-yl)acetic acid (0.027 g, 0.082 mmol, 10.2% yield). ¹H NMR (CDCl₃/400 MHz) δ (ppm) 7.15-7.19 (m, 2H), 6.97-7.04 (m, 3H), 4.60 (s, 2H), 2.47 (t, 2H, J=6.4 Hz), 2.23 (s, 3H), 2.16 (s, 2H), 1.59 (t, 2H, J=6.4 Hz), 0.92 (s, 6H).

### Example 56B

### Preparation of ethyl 2-(3-(4-(chlorosulfonyl)phenylthio)-2,5,5-trimethyl-4,5,6,7-tetrahydro-1H-indol-1-yl)acetate

Ethyl 2-(2,5,5-trimethyl-3-(phenylthio)-4,5,6,7-tetrahydro-1H-indol-1-yl)acetate (0.180 g, 0.503 mmol) was dissolved in DCM (3 ml) and stirred at 0° C. Chlorosulfonic acid (0.059 g, 0.503 mmol) was added dropwise to immediately afford a deep purple color. The reaction was stirred at 0° C. for 30 minutes and then warmed to room temperature and stirred another 30 minutes. To quench the reaction, the mixture was poured onto ice water and swirled by hand in an Erlenmeyer flask. Ethyl acetate was poured on top of this and the whole mixture poured into an extraction funnel. The aqueous layer was extracted 3× with EtOAc and the combined organic layer was washed once with brine and dried over sodium sulfate. The solution was concentrated in vacuo and used without any further purification.

### Example 56C

### Preparation of ethyl 2-(2,5,5-trimethyl-3-(4-(morpholinosulfonyl)phenylthio)-4,5,6,7-tetrahydro-1H-indol-1-yl)acetate (I-71)

The crude reaction mixture containing ethyl 2-(3-(4-(chlorosulfonyl)phenylthio)-2,5,5-trimethyl-4,5,6,7-tetrahydro-1H-indol-1-yl)acetate (0.288 g, 0.632 mmol) was taken up in DCM (3 ml) and stirred at room temperature. Morpholine (0.220 ml, 2.53 mmol) was added dropwise. The reaction was stirred for 12 hours and then quenched with brine. The aqueous layer was extracted with ethyl acetate, over sodium sulfate and concentrated in vacuo. The crude mixture was purified by silica gel chromatography using ethyl acetate and hexanes as eluents. A mixture of products was taken on to the next step without further purification.

### Example 56D

### Preparation of 2-(2,5,5-trimethyl-3-(4-(morpholinosulfonyl)phenylthio)-4,5,6,7-tetrahydro-1H-indol-1-yl)acetic acid (I-37)

Ethyl 2-(2,5,5-trimethyl-3-(4-(morpholinosulfonyl)phenylthio)-4,5,6,7-tetrahydro-1H-indol-1-yl)acetate (0.025 g, 0.049 mmol) was dissolved in a 1:1 mixture of THF (1 ml):Water (1.000 ml) and stirred at room temperature. Sodium hydroxide (0.148 ml, 0.148 mmol) 1M was added and the reaction was stirred for 12 hours. HCl was added to quench the reaction. After stirring for 10 minutes the reaction mixture was concentrated in vacuo. The residue was taken up in acetonitrile and purified by reverse phase HPLC to afford the desired product, 2-(2,5,5-trimethyl-3-(4-(morpholinosulfonyl)phenylthio)-4,5,6,7-tetrahydro-1H-indol-1-yl)acetic acid (0.013 g, 0.027 mmol, 55.0% yield). ¹H NMR (CDCl₃/400 MHz) δ (ppm) 7.51 (d, 2H, J=8.4 Hz), 7.08 (d, 2H, J=8.8 Hz), 4.62 (s, 2H), 3.74 (t, 4H, J=4.8 Hz), 2.97 (t, 4H, J=4.8 Hz), 2.47 (t, 2H, J=6.0 Hz), 2.12 (s, 2H), 1.60 (t, 2H, J=6.4 Hz), 0.93 (s, 6H).

### Example 57

### Preparation of ethyl 2-(2,5,5-trimethyl-3-(4-(pyrrolidin-1-ylsulfonyl)phenylthio)-4,5,6,7-tetrahydro-1H-indol-1-yl)acetate (I-72)

Ethyl 2-(3-(4-(chlorosulfonyl)phenylthio)-2,5,5-trimethyl-4,5,6,7-tetrahydro-1H-indol-1-yl)acetate (0.213 g, 0.467 mmol) was dissolved in DCM (3 ml) and stirred at room temperature to afford a green colored solution. Pyrrolidine (0.135 ml, 1.635 mmol) was added dropwise over 5 minutes. As addition and stirring occur, the reaction turns yellow. After two hours, the reaction mixture was adsorbed directly onto silica gel and purified by silica gel chromatography using ethyl acetate and hexanes. Concentration in vacuo of the desired fractions afforded ethyl 2-(2,5,5-trimethyl-3-(4-(pyrrolidin-1-ylsulfonyl)phenylthio)-4,5,6,7-tetrahydro-1H-indol-1-yl)acetate (0.053 g, 0.108 mmol, 23.12% yield). ¹H NMR (CDCl₃/400 MHz) δ (ppm) 7.58 (d, 2H, J=8.8 Hz), 7.06 (d, 2H, J=8.8 Hz), 4.54 (s, 2H), 4.23 (q, 2H, J=7.2 Hz), 3.18-3.22 (m, 4H), 2.45 (t, 2H, J=6.4 Hz), 2.19 (s, 3H), 2.09 (s, 2H), 1.73-1.76 (m, 4H), 1.58 (t, 2H, J=6.4 Hz), 1.29 (t, 3H, J=7.2 Hz), 0.91 (s, 6H).

### Example 58

### Preparation of 2-(2,5,5-trimethyl-3-(4-(pyrrolidin-1-ylsulfonyl)phenylthio)-4,5,6,7-tetrahydro-1H-indol-1-yl)acetic acid (I-38)

Ethyl 2-(2,5,5-trimethyl-3-(4-(pyrrolidin-1-ylsulfonyl)phenylthio)-4,5,6,7-tetrahydro-1H-indol-1-yl)acetate (0.053 g, 0.108 mmol) was dissolved in a 1:1 mixture of THF (1 ml) and Water (1.000 ml) and stirred at room temperature. Sodium hydroxide (0.324 ml, 0.324 mmol) was added and the reaction stirred for 12 hours. The solution was quenched with 3M HCl (150 ul) and stirred for 10 minutes. The was concentrated in vacuo to afford a greenish/yellow solid. The crude material was purified by reverse phase HPLC chromatography using acetonitrile and water (both spiked with TFA) as eluents to afford desired product 2-(2,5,5-trimethyl-3-(4-(pyrrolidin-1-ylsulfonyl)phenylthio)-4,5,6,7-tetrahydro-1H-indol-1-yl)acetic acid (0.034 g, 0.073 mmol, 68.0% yield). ¹H NMR (CDCl₃/400 MHz) δ (ppm) 7.59 (d, 2H, J=8.4 Hz), 7.06 (d, 2H, J=8.8 Hz), 4.63 (s, 2H), 3.19-3.23 (m, 4H), 2.47 (t, 2H, J=6.4 Hz), 2.21 (s, 3H), 2.10 (s, 2H), 1.74-1.77 (m, 4H), 1.59 (t, 2H, J=6.4 Hz), 0.92 (s, 6H).

### Example 59

### Preparation of ethyl 2-(2,6,6-trimethyl-3-(4-(morpholinosulfonyl)phenylthio)-4-oxo-4,5,6,7-tetrahydro-1H-indol-1-yl)acetate (I-69) and 2-(2,6,6-trimethyl-3-(4-(morpholinosulfonyl)phenylthio)-4-oxo-4,5,6,7-tetrahydro-1H-indol-1-yl)acetic acid (I-41)

### Example 59A

Preparation of 2,6,6-trimethyl-3-(phenylthio)-6,7-dihydro-1H-indol-4(5H)-one. To a stirring solution of 2,6,6-trimethyl-6,7-dihydro-1H-indol-4(5H)-one (0.560 g, 3.16 mmol) in N,N-dimethylformamide (7 ml) was added thiophenol (0.38 ml, 3.79 mmol) followed by iodine (0.96 g, 3.79 mmol). The vial was capped heated to 13° C. for 12 hours. It was then quenched with brine and extracted with ethyl acetate. The combined organic layer was washed brine to remove DMF and then dried over sodium sulfate. After concentration, the crude mixture was purified by silica gel chromatography to afford the desired product, 2,6,6-trimethyl-3-(phenylthio)-6,7-dihydro-1H-indol-4(5H)-one as a pale, yellow solid (0.340 g, 1.191 mmol, 37.7% yield). ¹H NMR (CDCl₃/400 MHz) δ (ppm) 7.14-7.18 (m, 2H), 7.07-7.09 (m, 2H), 6.99-7.05 (m, 1H), 2.66 (s, 2H), 2.32 (s, 2H), 2.27 (s, 3H), 1.11 (s, 6H).

### Example 59B

Preparation of ethyl 2-(2,6,6-trimethyl-4-oxo-3-(phenylthio)-4,5,6,7-tetrahydro-1H-indol-1-yl)acetate. To a stirring solution of 2,6,6-trimethyl-3-(phenylthio)-6,7-dihydro-1H-indol-4(5H)-one (0.34 g, 1.19 mmol) in acetone (10 ml) was added ethyl 2-bromoacetate (0.19 ml, 1.78 mmol) and potassium carbonate (0.49 g, 3.57 mmol) were added. The reaction was refluxed at 65° C. for 12 hours after which the reaction mixture was concentrated and purified by silica gel chromatography. The product, ethyl 2-(2,6,6-trimethyl-4-oxo-3-(phenylthio)-4,5,6,7-tetrahydro-1H-indol-1-yl)acetate, was isolated as a yellow, viscous oil (0.264 g, 0.711 mmol, 59.7% yield). ¹H NMR (CDCl₃/400 MHz) δ (ppm) 7.12-7.17 (m, 2H), 6.99-7.07 (m, 3H), 4.58 (s, 2H), 4.26 (q, 2H, J=6.8 Hz), 2.57 (s, 2H), 2.33 (s, 3H), 1.30 (t, 3H, J=7.2 Hz), 1.11 (s, 6H).

### Example 59C

Preparation of ethyl 2-(2,6,6-trimethyl-3-(4-(morpholinosulfonyl)phenylthio)-4-oxo-4,5,6,7-tetrahydro-1H-indol-1-yl)acetate (I-69). Ethyl 2-(2,6,6-trimethyl-4-oxo-3-(phenylthio)-4,5,6,7-tetrahydro-1H-indol-1-yl)acetate (0.26 g, 0.71 mmol) was dissolved in dichloromethane (5 ml) and stirred at 0° C. Chlorosulfonic acid (0.28 ml, 4.26 mmol) was added dropwise, resulting in a reaction color from yellow to brown. The reaction was slowly allowed to reach room temperature, stirred for 5 hours after which it was quenched by pouring onto ice and reversion back to its original yellow reaction color. Initial extraction with dichloromethane resulted in an emulsion. Re-extraction with ethyl acetate, followed by washing with brine and drying over sodium sulfate and concentration in vacuo and afforded a crude reaction mixture containing ethyl 2-(3-(4-(chlorosulfonyl)phenylthio)-2,6,6-trimethyl-4-oxo-4,5,6,7-tetrahydro-1H-indol-1-yl)acetate (0.283 g, 0.602 mmol, 85% yield) which was used without further purification. A solution of ethyl 2-(3-(4-(chlorosulfonyl)phenylthio)-2,6,6-trimethyl-4-oxo-4,5,6,7-tetrahydro-1H-indol-1-yl)acetate (0.14 g, 0.30 mmol was dissolved in dichloromethane (6 ml) was treated with morpholine (0.08 ml, 0.90 mmol) at rt. The reaction was allowed to stir at room temperature for 12 hours after which it was purified by silica gel chromatography using ethyl acetate and hexanes as eluents. The product, ethyl 2-(2,6,6-trimethyl-3-(4-(morpholinosulfonyl)phenylthio)-4-oxo-4,5,6,7-tetrahydro-1H-indol-1-yl)acetate (0.098 g, 0.188 mmol, 62.7% yield) was isolated as a clear oil. ¹H NMR (CDCl₃/400 MHz) δ (ppm) 7.51 (d, 2H, J=8.8 Hz), 7.13 (d, 2H, J=8.8 Hz), 4.61 (s, 2H), 4.27 (q, 2H, J=6.8 Hz), 3.70-3.73 (m, 4H), 2.96 (t, 4H, J=4.8 Hz), 2.60 (s, 2H), 2.35 (s, 2H), 2.22 (s, 3H), 1.32 (t, 3H, J=6.8 Hz), 1.13 (s, 6H).

### Example 59D

Preparation of 2-(2,6,6-trimethyl-3-(4-(morpholinosulfonyl)phenylthio)-4-oxo-4,5,6,7-tetrahydro-1H-indol-1-yl)acetic acid (I-41). Ethyl 2-(2,6,6-trimethyl-3-(4-(morpholinosulfonyl)phenylthio)-4-oxo-4,5,6,7-tetrahydro-1H-indol-1-yl)acetate (0.02 g, 0.05 mmol) was dissolved in a 1:1 mixture of THF (0.5 ml) and Water (0.50 ml). A 1 molar solution of sodium hydroxide (0.11 ml, 0.11 mmol) was added and stirred after which the reaction was quenched with 6 equiv of 3M HCl and stirred for 10 minutes. The crude mixture was concentrated in vacuo and taken up in MeOH and filtered through a syringe. Purification was achieved by semi-preparative reverse phase HPLC using 5% to 95% acetonitrile (with 0.1% trifluoroacetic acid additive) in water (with 0.1% trifluoroacetic acid over 70 minutes. The product 2-(2,6,6-trimethyl-3-(4-(morpholinosulfonyl)phenylthio)-4-oxo-4,5,6,7-tetrahydro-1H-indol-1-yl)acetic acid (0.0175 g, 0.036 mmol, 63.8% yield) was afforded as a white solid (0.0175 g, 64%). ¹H NMR (CDCl₃/400 MHz) δ (ppm) 7.51 (d, 2H, J=8.4 Hz), 7.12 (d, 2H, J=8.4 Hz), 4.69 (s, 2H), 3.75 (t, 4H, J=4 Hz), 2.97 (s, 4H), 2.63 (s, 2H), 2.42 (s, 2H), 2.25 (s, 3H), 1.14 (s, 6H).

### Example 60

### Preparation of ethyl 2-(2,6,6-trimethyl-4-oxo-3-(4-(pyrrolidin-1-ylsulfonyl)phenylthio)-4,5,6,7-tetrahydro-1H-indol-1-yl)acetate (I-70) and 2-(2,6,6-trimethyl-4-oxo-3-(4-(pyrrolidin-1-ylsulfonyl)phenylthio)-4,5,6,7-tetrahydro-1H-indol-1-yl)acetic acid (I-42).

### Example 60A

Preparation of ethyl 2-(2,6,6-trimethyl-4-oxo-3-(4-(pyrrolidin-1-ylsulfonyl)phenylthio)-4,5,6,7-tetrahydro-1H-indol-1-yl)acetate (I-70). Ethyl 2-(2,6,6-trimethyl-4-oxo-3-(phenylthio)-4,5,6,7-tetrahydro-1H-indol-1-yl)acetate (0.264 g, 0.711 mmol) was dissolved in dichloromethane (5 ml) and stirred at 0° C. Chlorosulfonic acid (0.283 ml, 4.26 mmol) was added dropwise, resulting in a color change from yellow to brown. The reaction was slowly allowed to reach room temperature stirred for 5 hours, after which it was quenched by pouring onto ice, resulting in considerable fuming and a reversion to the original yellow color. Initial extraction with dichloromethane resulted in an emulsion. Re-extraction with ethyl acetate, followed by washing with brine and drying over magnesium sulfate and concentration afforded a mixture containing ethyl 2-(3-(4-(chlorosulfonyl)phenylthio)-2,6,6-trimethyl-4-oxo-4,5,6,7-tetrahydro-1H-indol-1-yl)acetate (0.283 g, 0.602 mmol, 85% yield) which was used in the next step without further purification. Ethyl 24344-(chlorosulfonyl)phenylthio)-2,6,6-trimethyl-4-oxo-4,5,6,7-tetrahydro-1H-indol-1-yl)acetate (0.14 g, 0.30 mmol) was dissolved in dichloromethane (3 ml) and stirred. Pyrrolidine (0.07 ml, 0.90 mmol) was added. The reaction vessel was capped and stirred for 12 hours. Purification was achieved by silica gel chromatography using ethyl acetate and hexanes as eluents. The ethyl 2-(2,6,6-trimethyl-4-oxo-3-(4-(pyrrolidin-1-ylsulfonyl)phenylthio)-4,5,6,7-tetrahydro-1H-indol-1-yl)acetate (0.086 g, 0.170 mmol, 56.8% yield) was isolated as a viscous yellow oil. ¹H NMR (CDCl₃/400 MHz) δ (ppm) 7.57 (d, 2H, J=8.0 Hz), 7.09 (d, 2H, J=8.4 Hz), 4.61 (s, 2H), 4.27 (q, 2H, J=7.2 Hz), 3.18 (dd, 4H, J=6.8 Hz, J=6.8 Hz), 2.59 (s, 2H), 2.34 (s, 2H), 2.20 (s, 3H), 1.75 (dt, 4H, J=6.8 Hz, J=3.6 Hz), 1.31 (t, 3H, J=7.2 Hz), 1.1 (s, 6H).

### Example 60B

Preparation of 2-(2,6,6-trimethyl-4-oxo-3-(4-(pyrrolidin-1-ylsulfonyl)phenylthio)-4,5,6,7-tetrahydro-1H-indol-1-yl)acetic acid (I-42). Ethyl 2-(2,6,6-trimethyl-4-oxo-3-(4-(pyrrolidin-1-ylsulfonyl)phenylthio)-4,5,6,7-tetrahydro-1H-indol-1-yl)acetate (0.025 g, 0.050 mmol) was dissolved in a 1:1 mixture of THF (1 ml) and water (1 ml). A 1M aqueous solution of sodium hydroxide (0.149 ml, 0.149 mmol) was added and the reaction stirred for 12 hours after which the reaction showed consumption of starting material and formation of product. The reaction was quenched with 0.1 ml of 3M HCl for 20 minutes. The reaction mixture was concentrated in vacuo. The mixture was reconstituted in methanol and purified by semi-preparative reverse phase HPLC using a 5% to 95% gradient of acetonitrile (spiked with 0.1% TFA) and water (spiked with 0.1% TFA). The product, 2-(2,6,6-trimethyl-4-oxo-3-(4-(pyrrolidin-1-ylsulfonyl)phenylthio)-4,5,6,7-tetrahydro-1H-indol-1-yl)acetic acid (0.015 g, 0.03 mmol, 64% yield) was afforded as a solid. ¹H NMR (CDCl₃/400 MHz) δ (ppm) 7.57 (d, 2H, J=8.4 Hz), 7.07 (d, 2H, J=8.4 Hz), 4.67 (s, 2H), 3.18 (t, 4H, J=6.8 Hz), 2.62 (s, 2H), 2.40 (s, 2H), 2.21 (s, 3H), 1.74 (t, 4H, J=6.8 Hz), 1.12 (s, 6H).

### Example 61

Preparation of ethyl 2-(2,6,6-trimethyl-3-(4-(morpholinosulfonyl)phenylthio)-4,5,6,7-tetrahydro-1H-indol-1-yl)acetate (I-73). Ethyl 2-(2,6,6-trimethyl-3-(4-(morpholinosulfonyl)phenylthio)-4-oxo-4,5,6,7-tetrahydro-1H-indol-1-yl)acetate (0.0786 g, 0.151 mmol) was dissolved in THF and stirred at rt. Borane-THF (0.302 ml, 0.302 mmol) was added dropwise over 5 minutes and stirred for two hours to afford a clear homogeneous solution. EtOH was added to quench borane and brine was added. The solution was stirred for 10 minutes. The aqueous layer was extracted with ethyl acetate 3×. The combined organic layer was washed with brine and dried over sodium sulfate. The solution was concentrated in vacuo and purified by silica gel chromatography, using ethyl acetate and hexanes as eluents. The desired product ethyl 2-(2,6,6-trimethyl-3-(4-(morpholinosulfonyl)phenylthio)-4,5,6,7-tetrahydro-1H-indol-1-yl)acetate (0.0275 g, 0.054 mmol, 36.0% yield) was obtained. ¹H NMR (CDCl₃/400 MHz) δ (ppm) 7.51 (d, 2H, J=8.8 Hz), 7.09 (d, 2H, J=8.4 Hz), 4.52 (s, 2H), 4.24 (q, 2H, J=7.2 Hz), 3.73 (t, 4H, J=5.2 Hz), 2.96 (t, 4H, J=4.4 Hz), 2.31 (t, 2H, J=6.0 Hz), 2.23 (s, 2H), 2.19 (s, 3H), 1.47 (t, 2H, J=6.0 Hz), 1.30 (t, 3H, J=7.6 Hz), 1.00 (s, 6H).

### Example 62

Preparation of 2-(2,6,6-trimethyl-3-(4-(morpholinosulfonyl)phenylthio)-4,5,6,7-tetrahydro-1H-indol-1-yl)acetic acid (I-47). Ethyl 2-(2,6,6-trimethyl-3-(4-(morpholinosulfonyl)phenylthio)-4,5,6,7-tetrahydro-1H-indol-1-yl)acetate (0.0275 g, 0.054 mmol) was dissolved in a 1:1 mixture of THF (0.5 ml) and Water (0.5 ml) and stirred at room temperature. Sodium hydroxide (0.163 ml, 0.163 mmol) was added to afford a clear solution and was stirred for 12 hours. The reaction was quenched with 3M HCl 0.2 ml and stirred for 10 minutes. The reaction mixture was concentrated and reconstituted in methanol. The crude mixture was purified by reverse phase HPLC chromatography using acetonitrile and water as eluents (each spiked with TFA) to afford the desired product: 2-(2,6,6-trimethyl-3-(4-(morpholinosulfonyl)phenylthio)-4,5,6,7-tetrahydro-1H-indol-1-yl)acetic acid (0.0231 g, 0.048 mmol, 89% yield). ¹H NMR (CDCl₃/400 MHz) δ (ppm) 7.51 (d, 2H, J=8.4 Hz), 7.09 (d, 2H, J=8.8 Hz), 4.61 (s, 2H), 3.74 (t, 4H, J=4.8 Hz), 2.97 (t, 4H, J=4.8 Hz), 2.31 (t, 2H, J=6.4 Hz), 2.25 (s, 2H), 2.22 (s, 3H), 1.48 (t, 2H, J=6.4 Hz), 1.00 (s, 6H).

### Example 63

Preparation of ethyl 2-(2,6,6-trimethyl-3-(4-(pyrrolidin-1-ylsulfonyl)phenylthio)-4,5,6,7-tetrahydro-1H-indol-1-yl)acetate (I-71). Ethyl 2-(2,6,6-trimethyl-4-oxo-3-(4-(pyrrolidin-1-ylsulfonyl)phenylthio)-4,5,6,7-tetrahydro-1H-indol-1-yl)acetate (0.069 g, 0.137 mmol) was dissolved in THF (2 ml) and stirred at room temperature. Borane-THF (0.342 ml, 0.342 mmol) was added at and stirred for 2 h. Ethanol and brine were added to quench reaction. The solution was stirred 10 minutes. Ethyl acetate was used to extract aqueous layer 3×. The combined organics were washed with brine, dried over sodium sulfate and concentrated in vacuo. The resulting organic residue was purified by silica gel chromatography using ethyl acetate and hexanes as eluents. The desired product, ethyl 2-(2,6,6-trimethyl-3-(4-(pyrrolidin-1-ylsulfonyl)phenylthio)-4,5,6,7-tetrahydro-1H-indol-1-yl)acetate (0.010 g, 0.020 mmol, 14.91% yield) was afforded. ¹H NMR (CDCl₃/400 MHz) δ (ppm) 7.59 (d, 2H, J=8.4 Hz), 7.07 (d, 2H, J=8.4 Hz), 4.51 (s, 2H), 4.23 (q, 2H, J=7.2 Hz), 3.18-3.21 (m, 4H), 2.29 (t, 2H, J=6.0 Hz), 2.22 (s, 2H), 2.19 (s, 3H), 1.73-1.76 (m, 4H), 1.46 (t, 2H, J=6.4 Hz), 1.29 (t, 3H, J=7.2 Hz), 0.99 (s, 6H).

### Example 64

Preparation of 2-(2,6,6-trimethyl-3-(4-(pyrrolidin-1-ylsulfonyl)phenylthio)-4,5,6,7-tetrahydro-1H-indol-1-yl)acetic acid (I-46). Ethyl 2-(2,6,6-trimethyl-3-(4-(pyrrolidin-1-ylsulfonyl)phenylthio)-4,5,6,7-tetrahydro-1H-indol-1-yl)acetate (0.010 g, 0.020 mmol) was stirred in a 1:1 mixture of THF (1 ml) and Water (1.000 ml). Sodium hydroxide (0.061 ml, 0.061 mmol) was added and the reaction mixture was stirred for 12 hours. The reaction was quenched with 0.1 ml 3M HCl and stirred for 10 minutes. The mixture was concentrated in vacuo. The crude mixture was purified by reverse phase HPLC chromatography using water and acetonitrile (spiked with TFA) as eluents to afford desired product 2-(2,6,6-trimethyl-3-(4-(pyrrolidin-1-ylsulfonyl)phenylthio)-4,5,6,7-tetrahydro-1H-indol-1-yl)acetic acid (9.33 mg, 0.020 mmol, 99% yield). ¹H NMR (CDCl₃/400 MHz) δ (ppm) 7.60 (d, 2H, J=8.8 Hz), 7.06 (d, 2H, J=8.8 Hz), 4.60 (s, 2H), 3.19-3.22 (m, 4H), 2.30 (t, 2H, J=6.4 Hz), 2.21 (s, 3H), 1.74-1.77 (m, 4H, J=3.2 Hz), 1.47 (t, 2H, J=6.4 Hz), 1.00 (s, 6H).

### Example 65

### Preparation of ethyl 2-(2,6,6-trimethyl-3-(4-methyl-2-(pyrrolidin-1-ylsulfonyl)phenylthio)-4-oxo-4,5,6,7-tetrahydro-1H-indol-1-yl)acetate (I-54)

### Example 65A

Preparation of 2,6,6-trimethyl-3-(p-tolylthio)-6,7-dihydro-1H-indol-4(5H)-one. To a solution of 2,6,6-trimethyl-6,7-dihydro-1H-indol-4(5H)-one (0.29 g, 1.67 mmol) ind N,N-dimethylformamide (7 ml) was added 4-methylbenzenethiol (0.245 g, 2.00 mmol) and iodine (0.68 g, 2.67 mmol) were added. The vial was capped and heated at 120° C. for 12 hours. The reaction was quenched with the addition of brine and diluted with EtOAc. The aqueous layer was extracted with EtOAc. The combined organic layer was washed with brine and dried over sodium sulfate. After concentration in vacuo, the reaction was purified via silica gel chromatography using ethyl acetate and hexanes as eluents, which afforded desired product was obtained in good yield: 2,6,6-trimethyl-3-(p-tolylthio)-6,7-dihydro-1H-indol-4(5H)-one (0.262 g, 0.875 mmol, 52.4% yield). ¹H NMR (CDCl₃/400 MHz) δ (ppm) 6.91-7.03 (m, 4H), 2.65 (s, 2H), 2.33 (s, 2H), 2.27 (s, 3H), 2.24 (s, 3H), 1.10 (s, 6H).

### Example 65B

Preparation of ethyl 2-(2,6,6-trimethyl-4-oxo-3-(p-tolylthio)-4,5,6,7-tetrahydro-1H-indol-1-yl)acetate. To a solution of 2,6,6-trimethyl-3-(p-tolylthio)-6,7-dihydro-1H-indol-4(5H)-one (0.26 g, 0.87 mmol) dissolved in N,N-dimethylformamide (8 ml) stirring at room temperature was added ethyl bromoacetate (0.12 ml, 1.14 mmol) and sodium hydride (0.05 g, 1.40 mmol). The reaction was stirred for 3 h after which it was quenched with brine and diluted with ethyl acetate and stirred for 20 minutes. Two layers were separated. The aqueous layer was extracted with EtOAc. The combined organic layer was washed with brine, dried over sodium sulfate and concentrated. The crude material was purified by silica gel chromatography using ethyl acetate and hexanes as eluents to afford the desired product, ethyl 2-(2,6,6-trimethyl-4-oxo-3-(p-tolylthio)-4,5,6,7-tetrahydro-1H-indol-1-yl)acetate (0.268 g, 0.695 mmol, 79% yield). ¹H NMR (CDCl₃/400 MHz) δ (ppm) 6.94-6.99 (m, 4H), 4.57 (s, 2H), 4.24 (q, 2H, J=6.8 Hz), 2.55 (s, 2H), 2.30 (s, 2H), 2.22 (s, 3H), 2.21 (s, 3H), 1.28 (t, 3H, J=6.8 Hz), 1.1 (s, 6H).

### Example 65C

Preparation of ethyl 2-(3-(2-(chlorosulfonyl)-4-methylphenylthio)-2,6,6-trimethyl-4-oxo-4,5,6,7-tetrahydro-1H-indol-1-yl)acetate. Ethyl 2-(2,6,6-trimethyl-4-oxo-3-(p-tolylthio)-4,5,6,7-tetrahydro-1H-indol-1-yl)acetate (0.268 g, 0.695 mmol) was dissolved in dichloromethane (5 ml) and stirred at room temperature. After cooling to 0° C., chlorosulfonic acid (0.279 ml, 4.17 mmol) was added and the reaction immediately turned from a yellow color to a dark brown color. The reaction was slowly warmed to room temperature and stirred for 12 hours. The reaction mixture was poured onto ice and diluted with brine and ethyl acetate. After extraction and drying over sodium sulfate, the crude mixture was concentrated in vacuo to afford a brown solid which included ethyl 2-(3-(2-(chlorosulfonyl)-4-methylphenylthio)-2,6,6-trimethyl-4-oxo-4,5,6,7-tetrahydro-1H-indol-1-yl)acetate, which was used in the next step without further purification.

### Example 65D

Preparation of ethyl 2-(2,6,6-trimethyl-3-(4-methyl-2-(pyrrolidin-1-ylsulfonyl)phenylthio)-4-oxo-4,5,6,7-tetrahydro-1H-indol-1-yl)acetate (I-54). Ethyl 2-(3-(2-(chlorosulfonyl)-4-methylphenylthio)-2,6,6-trimethyl-4-oxo-4,5,6,7-tetrahydro-1H-indol-1-yl)acetate (0.336 g, 0.695 mmol) was dissolved in dichloromethane (5 ml) and stirred at room temperature. Pyrrolidine (0.345 ml, 4.17 mmol) was slowly added via syringe and the reaction allowed to stir open to the air. The reaction was capped and stirred for 12 hours after which the reaction was concentrated in vacuo. Purified by semi-preparative reverse phase HPLC using water and acetonitrile (each pre-treated with 0.1% TFA) afforded desired product, ethyl 2-(2,6,6-trimethyl-3-(4-methyl-2-(pyrrolidin-1-ylsulfonyl)phenylthio)-4-oxo-4,5,6,7-tetrahydro-1H-indol-1-yl)acetate (0.082 g, 0.158 mmol, 22.75% yield) as a brown solid. ¹H NMR (CDCl₃/400 MHz) δ (ppm) 7.74 (d, 1H, J=1.6 Hz), 7.00 (dd, 1H, J=8.0 Hz, J=1.2 Hz), 6.62 (d, 1H, J=8.0 Hz), 4.63 (s, 2H), 4.28 (q, 2H, J=7.2 Hz), 3.50 (t, 4H, J=6.8 Hz), 2.62 (s, 2H), 2.39 (s, 2H), 2.28 (s, 3H), 2.25 (s, 3H), 1.88-1.92 (m, 4H), 1.32 (t, 3H, J=7.2 Hz), 1.13 (s, 6H).

### Example 66

### Preparation of ethyl 2-(3-(4-bromo-2-(pyrrolidin-1-ylsulfonyl)phenylthio)-2,6,6-trimethyl-4-oxo-4,5,6,7-tetrahydro-1H-indol-1-yl)acetate (I-53)

### Example 66A

Preparation of 3-(4-bromophenylthio)-2,6,6-trimethyl-6,7-dihydro-1H-indol-4(5H)-one. To a stirring solution of 2,6,6-trimethyl-6,7-dihydro-1H-indol-4(5H)-one (0.520 g, 2.93 mmol) in N,N-dimethylformamide (9 ml) at room temperature was added 4-bromobenzenethiol (0.66 g, 3.52 mmol) and iodine (1.11 g, 4.40 mmol). The vial was capped and heated to 120° C. for 12 hours after which it was quenched with brine and diluted with ethyl acetate. The layers were separated and the aqueous layer was extracted with EtOAc. The combined organic layer was washed with brine and dried over sodium sulfate. After concentration the purple/brown residue was taken up in DCM and purified by silica gel chromatography using ethyl acetate and hexanes as eluents to afford desired material 3-(4-bromophenylthio)-2,6,6-trimethyl-6,7-dihydro-1H-indol-4(5H)-one (0.700 g, 1.921 mmol, 65.5% yield). ¹H NMR (CDCl₃/400 MHz) δ (ppm) 8.33 (br.s, 1H), 7.24-7.27 (m, 2H), 6.94 (2H, d, 1H, J=8.8 Hz), 2.65 (s, 2H), 2.32 (s, 2H), 2.27 (s, 3H), 1.11 (s, 6H).

### Example 66B

Preparation of ethyl 2-(3-(4-bromophenylthio)-2,6,6-trimethyl-4-oxo-4,5,6,7-tetrahydro-1H-indol-1-yl)acetate. To a stirring solution of 3-(4-bromophenylthio)-2,6,6-trimethyl-6,7-dihydro-1H-indol-4(5H)-one (0.7 g, 1.92 mmol) in N,N-dimethylformamide (10 ml) at room temperature was added ethyl bromoacetate (0.27 ml, 2.30 mmol) and sodium hydride (0.09 g, 2.30 mmol). The reaction was stirred for 2 hr at which time it was quenched with brine and diluted with ethyl acetate. The layers were separated. The aqueous layer was extracted with ethyl acetate. The combined organic layer was washed with brine and then washed over sodium sulfate. After concentration, the crude material was purified on silica gel chromatography using ethyl acetate and hexanes. The purified material, ethyl 24344-bromophenylthio)-2,6,6-trimethyl-4-oxo-4,5,6,7-tetrahydro-1H-indol-1-yl)acetate, was a viscous yellow oil (0.687 g, 1.525 mmol, 79% yield). ¹H NMR (CDCl₃/400 MHz) δ (ppm) 7.25-7.27 (m, 2H), 6.93 (d, 2H, J=8.8 Hz), 4.59 (s, 2H), 4.26 (q, 2H, J=7.2 Hz), 2.57 (s, 2H), 2.33 (s, 2H), 2.23 (s, 3H), 1.30 (t, 2H, J=7.2 Hz), 1.11 (s, 6H).

### Example 66C

Preparation of ethyl 2-(3-(4-bromo-2-(chlorosulfonyl)phenylthio)-2,6,6-trimethyl-4-oxo-4,5,6,7-tetrahydro-1H-indol-1-yl)acetate. Ethyl 2-(3-(4-bromophenylthio)-2,6,6-trimethyl-4-oxo-4,5,6,7-tetrahydro-1H-indol-1-yl)acetate (0.687 g, 1.53 mmol) was dissolved in dichloromethane (8 ml) and stirred at 0° C. Chlorosulfonic acid (0.613 ml, 9.15 mmol) was added, resulting in a deep brown colored solution. The reaction was stirred vigorously for 30 minutes at 0° C., then warmed to room temperature and stirred two hours. The reaction mixture was then elevated to 60° C. and maintained at that temperature. The reaction was then cooled to room temperature and quenched by pouring over ice water. The mixture was diluted in ethyl acetate and the two layers were separated. Extraction with ethyl acetate (3×), drying over sodium sulfate, washing with brine (1× vol) and concentration afforded a brown/black/purple heterogeneous that included ethyl 2-(3-(4-bromo-2-(chlorosulfonyl)phenylthio)-2,6,6-trimethyl-4-oxo-4,5,6,7-tetrahydro-1H-indol-1-yl)acetate. This mixture was used in the next step without further purification.

### Example 66D

Preparation of ethyl 2-(3-(4-bromo-2-(pyrrolidin-1-ylsulfonyl)phenylthio)-2,6,6-trimethyl-4-oxo-4,5,6,7-tetrahydro-1H-indol-1-yl)acetate (I-53). The crude mixture of ethyl 2-(3-(4-bromo-2-(chlorosulfonyl)phenylthio)-2,6,6-trimethyl-4-oxo-4,5,6,7-tetrahydro-1H-indol-1-yl)acetate (0.837 g, 1.53 mmol) was dissolved in dichloromethane (10 ml) and stirred at room temperature. Pyrrolidine (0.631 ml, 7.63 mmol) was added and the reaction was stirred for 12 hours. After 12 hours, the reaction was concentrated, dissolved in a few ml of DCM and purified by silica gel chromatography. Using 60% ethyl acetate in hexanes afforded a yellow oil. Further purification using semi-preparative HPLC reverse phase with a gradient of 5% to 95% acetonitrile (spiked with 0.1% TFA) in water (spiked with 0.1% TFA) afforded ethyl 2-(3-(4-bromo-2-(pyrrolidin-1-ylsulfonyl)phenylthio)-2,6,6-trimethyl-4-oxo-4,5,6,7-tetrahydro-1H-indol-1-yl)acetate (0.003 g, 5.14 μmol, 0.3% yield) as a white solid. ¹H NMR (CDCl₃/400 MHz) δ (ppm) 8.05 (d, 1H, J=2.4 Hz), 7.26 (dd, 2H, J=8.4 Hz, J=2.0 Hz), 6.24 (d, 1H, J=8.8 Hz), 4.62 (s, 2H), 4.28 (q, 2H, J=7.2 Hz), 3.55 (t, 4H, J=6.8 Hz), 2.59 (s, 2H), 2.29 (s, 2H), 2.24 (s, 3H), 1.92-1.96 (m, 4H), 1.32 (t, 3H, J=7.2 Hz), 1.11 (s, 6H).

### Example 67

Preparation of Ethyl 2-(3-(4-fluoro-2-(pyrrolidin-1-ylsulfonyl)phenylthio)-2,6,6-trimethyl-4-oxo-4,5,6,7-tetrahydro-1H-indol-1-yl)acetate (I-57). Ethyl 2-(3-(4-fluoro-2-(pyrrolidin-1-ylsulfonyl)phenylthio)-2,6,6-trimethyl-4-oxo-4,5,6,7-tetrahydro-1H-indol-1-yl)acetate was made in the same fashion as ethyl 2-(3-(4-bromo-2-(pyrrolidin-1-ylsulfonyl)phenylthio)-2,6,6-trimethyl-4-oxo-4,5,6,7-tetrahydro-1H-indol-1-yl)acetate except 4-fluorobenzenethiol was used in the coupling instead of 4-bromobenzenethiol. ¹H NMR (400 MHz, CDCl₃) δ (ppm): 7.68 (dd, 1H, J=8.4 Hz, J=2.8 Hz), 6.88-6.93 (m, 1H), 6.75 (dd, 1H, J=8.8 Hz, J=5.2 Hz), 4.61 (s, 2H), 4.28 (q, 2H, J=7.2 Hz), 3.55-3.59 (m, 4H), 2.58 (s, 2H), 2.27 (s, 2H), 2.26 (s, 3H), 1.93-1.96 (m, 4H), 1.32 (t, 3H, J=7.2 Hz), 1.1 (s, 6H).

### Example 68

Preparation of 2-(3-(hydroxy(2-(pyrrolidin-1-ylsulfonyl)phenyl)methyl)-2,6,6-trimethyl-4-oxo-4,5,6,7-tetrahydro-1H-indol-1-yl)acetic acid. (I-50). The title compound was synthesized in the same manner as ethyl 2-(2,6,6-trimethyl-4-oxo-3-(2-(pyrrolidin-1-ylsulfonyl)benzyl)-4,5,6,7-tetrahydro-1H-indol-1-yl)acetate (I-45) starting from 3-(hydroxy(2-(pyrrolidin-1-ylsulfonyl)phenyl)methyl)-2,6,6-trimethyl-6,7-dihydro-1H-indol-4(5H)-one. ¹H NMR (400 MHz, CDCl₃) δ (ppm): 12 (br s, 1H), 8.10 (d, 1H, J=9.2 Hz), 7.48 (m, 2H), 6.75 (d, 1H, J=8.0 Hz), 4.74 (s, 2H), 3.78 (m, 2H), 3.41-3.13 (m, 2H), 3.0 (m, 2H) 2.82-2.75 (m, 4H), 2.32 (s, 3H), 2.16 (m, 2H), 1.95 (m, 2H), 1.19 (s, 3H), 1.16 (s, 3H).

### Example 69

### Synthesis of: 2-(2,6,6-trimethyl-3-(N-methyl-N-phenylsulfamoyl)-4-oxo-4,5,6,7-tetrahydro-1H-indol-1-yl)acetic acid (I-75)

To a stirring solution of ethyl 2-(2,6,6-trimethyl-4-oxo-4,5,6,7-tetrahydro-1H-indol-1-yl)acetate (0.093 g, 0.353 mmol) in 2 mL of dichlorethane was added chlorosulfonic acid (0.142 ml, 2.119 mmol) at rt. The reaction fumed and turned a brown color. After stirring for 30 minutes LCMS showed formation of the sulfonic acid. The reaction temperature was raised to 50° C. and stirred for 20 minutes and the LCMS shows approximately 20% formation of the correct product and the reaction was allowed to stir overnight. After 18 hours the approximate conversion to desired product was 60% (there was a significant amount of starting material still present). The ethyl acetate mixture was poured onto ice in an Erlenmeyer flask, extracted with ethyl acetate, washed with brine and dried over sodium sulfate. After concentration in vacuo, the material was collected and dried on high vac for 20 minutes to afford a brown oil. Ethyl 2-(3-(chlorosulfonyl)-2,6,6-trimethyl-4-oxo-4,5,6,7-tetrahydro-1H-indol-1-yl)acetate was taken forward to the next step without further purification.

To a stirring solution of ethyl 2-(3-(chlorosulfonyl)-2,6,6-trimethyl-4-oxo-4,5,6,7-tetrahydro-1H-indol-1-yl)acetate (0.093 g, 0.257 mmol) in 2 mL of dichloroethane was added triethylamine (0.179 ml, 1.285 mmol) followed by N-methylaniline (0.084 ml, 0.771 mmol). The reaction color was brown. LCMS 10 minutes after shows formation of desired product. After 18 hours LCMS analysis indicated significant formation of desired product and the reaction mixture was purified directly by reverse phase HPLC. After collection, concentrated using toluene as an azeotrope and drying on high vac for 2 h, ethyl 2-(2,6,6-trimethyl-3-(N-methyl-N-phenylsulfamoyl)-4-oxo-4,5,6,7-tetrahydro-1H-indol-1-yl)acetate (0.028 g, 0.065 mmol, 25.2% yield) was isolated.

To a stirring solution of ethyl 2-(2,6,6-trimethyl-3-(N-methyl-N-phenylsulfamoyl)-4-oxo-4,5,6,7-tetrahydro-1H-indol-1-yl)acetate (0.028 g, 0.065 mmol) in a 1:1 mixture of THF/H2O was added sodium hydroxide (0.129 ml, 0.129 mmol). The reaction was stirred overnight at room temperature. LCMS next day shows completion of reaction. The reaction was quenched with 1N HCl, stirred for 10 minutes, then concentrated and taken up in a solution of water and THF. The mixture was transferred to a scintillation vial and placed under an argon stream, it was purified by reverse phase HPLC using acetonitrile and water both spiked with 0.1% TFA. The desired fractions were collected and concentrated in vacuo. The crude mixture was dissolved in dichloromethane and aqueous sodium bicarbonate and separated. The aqueous layer held desired material which was then treated with HCl(aq) and extracted with ethyl acetate (3×). The combined organic layers were washed with brine and dried over sodium sulfate and concentrated in vacuo. After drying, 2-(2,6,6-trimethyl-3-(N-methyl-N-phenylsulfamoyl)-4-oxo-4,5,6,7-tetrahydro-1H-indol-1-yl)acetic acid (0.0056 g, 0.014 mmol, 21.39% yield) was isolated as a white solid. ¹H NMR (400 MHz, CD₃OD) δ (ppm): 7.16-7.07 (m, 4H), 4.49 (s, 2H), 3.23 (s, 3H (covered by methanol peak), 2.56 (s, 2H), 2.27 (s, 2H) 1.86 (s, 3H), 1.10 (s, 6H).

### Example 70

### Preparation of 2-(2,5,5-trimethyl-3-(2-(morpholinosulfonyl)phenylthio)-4,5,6,7-tetrahydro-1H-indol-1-yl)acetic acid (I-76)

### Example 70A

### 4-(2-fluorophenylsulfonyl)morpholine

To a solution of 2-fluorobenzene-1-sulfonyl chloride (3.03 g, 15.6 mmol) in acetone (50 mL) was slowly added morpholine (2.71 ml, 31.1 mmol). The reaction became immediately white in color, leading to the formation of a precipitate. After stirring at room temperature for 14 hours, the reaction was diluted in water, extracted with diethyl ether (3×50 mL), washed with 1M hydrochloric acid solution (1×50 mL), saturated ammonium chloride solution (1×50 mL), dried (sodium sulfate), filtered and concentrated to afford 4-(2-fluorophenylsulfonyl)morpholine (3.32 g, 13.5 mmol, 87% yield) as a white solid.

### Example 70B

### 4-(2-(benzylthio)phenylsulfonyl)morpholine

A slurry of 4-(2-fluorophenylsulfonyl)morpholine (3.32 g, 13.5 mmol), potassium carbonate (2.17 g, 15.7 mmol) and benzyl mercaptan (1.60 mL, 13.5 mmol) was heated in N,N-dimethylformamide (5.4 mL) for 12 hours at 70° C. The reaction was only about 40% complete, so the temperature was raised to 100° C. for 48 hours after which the reaction was determined to be complete by LCMS analysis. The reaction mixture was filtered, extracted with dichloromethane (3×50 mL), washed with water (5×100 mL), dried (sodium sulfate), filtered and concentrated to afford 4-(2-(benzylthio)phenylsulfonyl)morpholine (4.02 g, 11.5 mmol, 85% yield) as a pink solid.

### Example 70C

### 4-(2-(benzylsulfinyl)phenylsulfonyl)morpholine

To a slurry of 4-(2-(benzylthio)phenylsulfonyl)morpholine (4.02 g, 11.5 mmol) in glacial acetic acid (40 mL) at 0° C. was added a solution of aqueous 50% hydrogen peroxide (0.895 ml, 14.6 mmol). The reaction was warmed to room temperature and stirred for 6 hours, after which the reaction was diluted in water (350 mL), leading to the formation of a pinkish-white precipitate. This solid was filtered and dried to afford 4-(2-(benzylsulfinyl)phenylsulfonyl)morpholine (3.61 g, 9.88 mmol, 86% yield) as a pale pinkish-white solid.

### Example 70D

### 1,2-bis(2-(morpholinosulfonyl)phenyl)disulfane

To a slurry of 4-(2-(benzylsulfinyl)phenylsulfonyl)morpholine (3.61 g, 9.88 mmol) in methanol (15 mL) was added concentrated hydrochloric acid solution (15 mL, 180 mmol). The slurry was refluxed for 5 hours, after which the mixture was cooled to room temperature and decanted. The remaining heavy residue was dissolved in dichloromethane and extracted with dichloromethane (3×50 mL), dried (sodium sulfate), filtered and concentrated to a residue which was purified on column chromatography (ISCO 120 g, 20 mL/min) using 5 to 75% ethyl acetate in hexanes over 75 minutes affording 1,2-bis(2-(morpholinosulfonyl)phenyl)disulfane (1.16 g, 2.25 mmol, 23% yield) which was isolated as a white solid.

### Example 70E

### ethyl 2-(2,5,5-trimethyl-4,5,6,7-tetrahydro-1H-indol-1-yl)acetate

The synthesis of this compound is outlined in the preparation of 2-(2,5,5-trimethyl-3-(2-(morpholinosulfonyl)benzyl)-4,5,6,7-tetrahydro-1H-indol-1-yl)acetic acid.

### Example 70F

### ethyl 2-(2,5,5-trimethyl-3-(2-(morpholinosulfonyl)phenylthio)-4,5,6,7-tetrahydro-1H-indol-1-yl)acetate

To a 0° C. solution of 1,2-bis(2-(morpholinosulfonyl)phenyl)disulfane (104 mg, 0.201 mmol) in N,N-dimethylformamide (1 mL) was added a solution of iodine (50.9 mg, 0.201 mmol) in N,N-dimethylformamide (1 mL). The reaction was stirred for 5 minutes at 0° C., after which a solution of ethyl 2-(2,5,5-trimethyl-4,5,6,7-tetrahydro-1H-indol-1-yl)acetate (50.0 mg, 0.201 mmol) in N,N-dimethylformamide (1 mL) was added. The reaction was warmed to room temperature, then heated to 60° C. for 2.5 hours, after which the reaction was complete by LCMS analysis. The reaction was diluted in water and extracted with ethyl acetate (3×30 mL), dried (sodium sulfate), filtered and concentrated to a brown residue. Purification was achieved by silica gel chromatography (ISCO 40 g, 20 mL/min), using 5 to 75% ethyl acetate in hexanes over 60 minutes, affording ethyl 2-(2,5,5-trimethyl-3-(2-(morpholinosulfonyl)phenylthio)-4,5,6,7-tetrahydro-1H-indol-1-yl)acetate (62.5 mg, 0.123 mmol, 62% yield) as a tan foam.

LCMS [ES]+: found 507.34 at 3.62 min.

### Example 70G

### 2-(2,5,5-trimethyl-3-(2-(morpholinosulfonyl)phenylthio)-4,5,6,7-tetrahydro-1H-indol-1-yl)acetic acid

To a solution of ethyl 2-(2,5,5-trimethyl-3-(2-(morpholinosulfonyl)phenylthio)-4,5,6,7-tetrahydro-1H-indol-1-yl)acetate (62.5 mg, 0.123 mmol) in tetrahydrofuran (3 mL) and water (3 mL) was added a 3M aqueous solution of sodium hydroxide (0.0820 ml, 0.247 mmol). The reaction was stirred at room temperature for 45 minutes, after which it was heated to 60° C. for 30 minutes. The reaction was about ⅔ complete at this point. Additional 3M aqueous sodium hydroxide (0.082 ml, 0.247 mmol) was added, and the reaction was stirred at 60° C. for 30 minutes, after which the reaction was determined to be complete by LCMS analysis. The mixture was concentrated to remove the tetrahydrofuran, neutralized with aqueous 6M hydrochloric acid solution (82 μL), extracted with ethyl acetate (3×30 mL), dried (sodium sulfate), filtered, and concentrated to afford 2-(2,5,5-trimethyl-3-(2-(morpholinosulfonyl)phenylthio)-4,5,6,7-tetrahydro-1H-indol-1-yl)acetic acid as a tan solid. 1H NMR (400 MHz, CDCl3) δ (ppm): 7.87 (dd, 1H, J=8.0 Hz, J=1.2 Hz), 7.24-7.27 (m, 1H), 7.12 (app t, 1H, J=7.6 Hz), 6.83 (d, 1H, J=8.4 Hz), 4.61 (s, 2H), 3.77 (m, 4H), 3.33 (m, 4H), 2.47 (t, 2H, J=6.0 Hz), 2.20 (s, 3H), 2.07 (s, 2H), 1.59 (t, 2H, J=6.4 Hz), 0.91 (s, 6H). LCMS [ES]+: found 479.28 at 3.25 min.

### Example 71

### Synthesis of: 2-(2-methyl-4-oxo-3-(2-(pyrrolidin-1-ylsulfonyl)benzyl)-4,5,6,7-tetrahydro-1H-indol-1-yl)acetic acid (I-77)

### Example 71A

### 2-methyl-6,7-dihydro-1H-indol-4(5H)-one

2-(2-oxopropyl)cyclohexane-1,3-dione (160 mg, 0.951 mmol) was dissolved in acetic acid (1.5 mL), after which acetic anhydride (0.188 mL, 2.00 mmol), and ammonium acetate (367 mg, 4.76 mmol) were added. The reaction was stirred at 75° C. for 12 hours, then cooled to room temperature, extracted with dichloromethane (4×30 mL), and extracted with 3N sodium hydroxide into the aqueous layer. The organic layer was then back-extracted with dichloromethane (3×30 mL), dried (sodium sulfate), filtered, and concentrated to a tan solid 2-methyl-6,7-dihydro-1H-indol-4(5H)-one (80 mg, 0.536 mmol, 56.4% yield).

### Example 71B

### 3-(hydroxy(2-(pyrrolidin-1-ylsulfonyl)phenyl)methyl)-2-methyl-6,7-dihydro-1H-indol-4(5H)-one

To a solution of 2-methyl-6,7-dihydro-1H-indol-4(5H)-one (125 mg, 0.838 mmol) in 2,2,2-trifluoroethanol (3 mL) was added 2-(pyrrolidin-1-ylsulfonyl)benzaldehyde (200 mg, 0.838 mmol). A 3M aqueous solution of sodium hydroxide (0.838 mL, 2.51 mmol) was added and stirred at 75° C. After 24 hours, the reaction was cooled to room temperature and diluted with water and dichloromethane. Sodium bisulfite (excess) was added and the reaction was stirred for 12 hours at room temperature. The layers were separated and the organic layer was extracted with dichloromethane (3×30 mL), dried (sodium sulfate), filtered and concentrated to a brown oil. First pass purification using silica gel (ISCO 25 g, 20 mL/min) afforded roughly a 1:1 mix of 2-methyl-6,7-dihydro-1H-indol-4(5H)-one and product (170 mg). Used in the next step without further purification.

### Example 71C

### 2-methyl-3-(2-(pyrrolidin-1-ylsulfonyl)benzyl)-6,7-dihydro-1H-indol-4(5H)-one

To a solution of 3-(hydroxy(2-(pyrrolidin-1-ylsulfonyl)phenyl)methyl)-2-methyl-6,7-dihydro-1H-indol-4(5H)-one (170 mg, 0.438 mmol) in dichloromethane (4 mL) was added triethylsilane (0.140 ml, 0.875 mmol) followed by trifluoroacetic acid (0.101 mL, 1.31 mmol). The reaction was stirred at room temperature for 30 minutes, after which analysis by LCMS indicated the presence of the reduced species. The reaction was extracted in dichloromethane (3×30 mL), dried (sodium sulfate), filtered and concentrated to a brown solid which was purified on silica gel (Luknova 25 g, 20 mL/min) using 10 to 100% ethyl acetate in hexanes afforded a 1:1 mixture of N and C-alkyl products. Repurification on silica gel using 1 to 10% of 7:1 acetonitrile/methanol in dichloromethane over 60 minutes afforded 2-methyl-3-(2-(pyrrolidin-1-ylsulfonyl)benzyl)-6,7-dihydro-1H-indol-4(5H)-one (32.0 mg, 0.0860 mmol, 20% yield).

### Example 71D

### ethyl 2-(2-methyl-4-oxo-3-(2-(pyrrolidin-1-ylsulfonyl)benzyl)-4,5,6,7-tetrahydro-1H-indol-1-yl)acetate (I-78)

To a slurry of 2-methyl-3-(2-(pyrrolidin-1-ylsulfonyl)benzyl)-6,7-dihydro-1H-indol-4(5H)-one (32 mg, 0.086 mmol), ethyl bromoacetate (0.0480 mL, 0.430 mmol), potassium carbonate (23.75 mg, 0.172 mmol), and potassium iodide (4.28 mg, 0.026 mmol) in Acetonitrile (Volume: 3 ml) was heated to 110° C. After 3 hours, the reaction was complete. The crude reaction mixture was applied directly to a silica gel column (Luknova 25 g, 20 mL/min) using 10 to 100% ethyl acetate in hexanes over 75 minutes. Fractions 51-60 afforded ethyl 2-(2-methyl-4-oxo-3-(2-(pyrrolidin-1-ylsulfonyl)benzyl)-4,5,6,7-tetrahydro-1H-indol-1-yl)acetate (29.7 mg, 0.0650 mmol, 75% yield) as an off white foam.

### Example 72

### Synthesis of: 2-(2,6,6-trimethyl-4-oxo-3-(2-(thiophen-2-ylsulfonyl)benzyl)-4,5,6,7-tetrahydro-1H-indol-1-yl)acetic acid (I-81)

### Example 72A

### 2-(thiophen-2-ylsulfonyl)benzaldehyde

A solution of sodium thiophene-2-sulfinate (1.00 g, 5.88 mmol) and 2-fluorobenzaldehyde (0.614 ml, 5.88 mmol) in DMSO (6 mL) was heated to 120° C. for 4 days. The reaction was cooled to room temperature, then extracted with dichloromethane (4×30 mL), dried (sodium sulfate), filtered and concentrated to afford a brown residue which was purified on silica gel (Luknova 80 g, 20 mL/min) to afford 2-(thiophen-2-ylsulfonyl)benzaldehyde (99 mg) as a solid.

### Example 72B

### 3-(hydroxy(2-(thiophen-2-ylsulfonyl)phenyl)methyl)-2,6,6-trimethyl-6,7-dihydro-1H-indol-4(5H)-one

To a solution of 2,6,6-trimethyl-6,7-dihydro-1H-indol-4(5H)-one (69.5 mg, 0.392 mmol) and 2-(thiophen-2-ylsulfonyl)benzaldehyde (99.0 mg, 0.392 mmol) in 2,2,2-trifluoroethanol (2 mL) was added a 1M solution of aqueous sodium hydroxide (1.18 mL, 1.18 mmol). The reaction was stirred at 65° C. for 12 hours, then heated at 100° C. for 24 hours. The reaction was cooled to room temperature, diluted with saturated ammonium chloride solution (20 mL), and extracted with dichloromethane (3×40 mL), dried (sodium sulfate), filtered and concentrated to a brown solid. Purification was achieved by silica gel chromatography (ISCO 40 g, 20 mL/min) using 10 to 100% ethyl acetate in hexanes over 55 minutes. The product 3-(hydroxy(2-(thiophen-2-ylsulfonyl)phenyl)methyl)-2,6,6-trimethyl-6,7-dihydro-1H-indol-4(5H)-one (99.0 mg, 0.230 mmol, 59% yield) was isolated as a gold-colored solid.

### Example 72C

### 2,6,6-trimethyl-3-(2-(thiophen-2-ylsulfonyl)benzyl)-6,7-dihydro-1H-indol-4(5H)-one

To a solution of 3-(hydroxy(2-(thiophen-2-ylsulfonyl)phenyl)methyl)-2,6,6-trimethyl-6,7-dihydro-1H-indol-4(5H)-one (99 mg, 0.230 mmol) in 4 mL of dichloromethane) was added neat trifluoroacetic acid (0.0530 mL, 0.691 mmol) followed by triethylsilane (0.0740 ml, 0.461 mmol). The reaction was heated to 70° C. for 5 minutes, after which it was shown to be complete by LCMS analysis. The reaction was cooled to room temperature and then neutralized by the addition of 1M sodium hydroxide solution (3 mL), extracted with dichloromethane (3×40 mL), dried (sodium sulfate), filtered and concentrated to 2,6,6-trimethyl-3-(2-(thiophen-2-ylsulfonyl)benzyl)-6,7-dihydro-1H-indol-4(5H)-one (73.9 mg, 0.179 mmol, 78% yield) as a pink solid. The material was used in the next step without further purification.

### Example 72D

### ethyl 2-(2,6,6-trimethyl-4-oxo-3-(2-(thiophen-2-ylsulfonyl)benzyl)-4,5,6,7-tetrahydro-1H-indol-1-yl)acetate (I-80)

A slurry of 2,6,6-trimethyl-3-(2-(thiophen-2-ylsulfonyl)benzyl)-6,7-dihydro-1H-indol-4(5H)-one (67 mg, 0.162 mmol), ethyl bromoacetate (0.0900 mL, 0.810 mmol), potassium iodide (8.1 mg, 0.049 mmol), and potassium carbonate (44.8 mg, 0.324 mmol) was heated in acetonitrile (3 mL) at 120° C. for 4 hours, after which the reaction was shown to be complete by LCMS analysis. The reaction was cooled to room temperature, then purified directly (loaded in the reaction solvent without any concentration) on silica gel (ISCO 40 g, 20 mL/min) using 10% ethyl acetate in hexanes (first 10 minutes) then a gradient to 60% ethyl acetate in hexanes over 75 minutes to afford ethyl 2-(2,6,6-trimethyl-4-oxo-3-(2-(thiophen-2-ylsulfonyl)benzyl)-4,5,6,7-tetrahydro-1H-indol-1-yl)acetate (66.0 mg, 0.132 mmol, 82% yield), which was isolated as a creme-colored foam.

### Example 72E

### 2-(2,6,6-trimethyl-4-oxo-3-(2-(thiophen-2-ylsulfonyl)benzyl)-4,5,6,7-tetrahydro-1H-indol-1-yl)acetic acid (I-81)

A solution of ethyl 2-(2,6,6-trimethyl-4-oxo-3-(2-(thiophen-2-ylsulfonyl)benzyl)-4,5,6,7-tetrahydro-1H-indol-1-yl)acetate (66.0 mg, 0.132 mmol) in THF (2 mL) and water (2 mL) was added 1M aqueous sodium hydroxide (0.264 ml, 0.264 mmol) solution. After 25 minutes, the reaction was complete by LCMS analysis. The reaction was concentrated and then neutralized by the addition of 44 μL of 6N aqueous hydrochloric acid solution. An off-white precipitate formed and was collected. The solid was washed with three volumes of water then dried in-vacuo to afford an off-white solid 2-(2,6,6-trimethyl-4-oxo-3-(2-(thiophen-2-ylsulfonyl)benzyl)-4,5,6,7-tetrahydro-1H-indol-1-yl)acetic acid (47.2 mg, 0.100 mmol, 76% yield). ¹H NMR (400 MHz, CDCl₃) δ (ppm): 8.05 (d, 1H, J=7.6 Hz), 7.68 (dd, 1H, J=4.0 Hz, J=1.2 Hz), 7.58 (dd, 1H, J=4.8 Hz, J=1.2 Hz), 7.19-7.27 (m, 2H), 7.01-7.03 (m, 1H), 6.88 (d, 1H, J=7.6 Hz), 4.42 (s, 4H), 2.47 (s, 2H), 2.16 (s, 2H), 1.98 (s, 3H), 1.00 (s, 6H).

### Example 73

### Synthesis of: 2-(2,6,6-trimethyl-4-oxo-3-(2-(phenylsulfonyl)benzyl)-4,5,6,7-tetrahydro-1H-indol-1-yl)acetic acid (I-48)

### Example 73A

### 3-(hydroxy(2-(phenylsulfonyl)phenyl)methyl)-2,6,6-trimethyl-6,7-dihydro-1H-indol-4(5H)-one

To a solution of 2,6,6-trimethyl-6,7-dihydro-1H-indol-4(5H)-one (114 mg, 0.642 mmol) and 2-(phenylsulfonyl)benzaldehyde (158 mg, 0.642 mmol) in 2,2,2-trifluoroethanol (1.28 mL) was added a 1M solution of aqueous sodium hydroxide (0.642 mL, 1.93 mmol). The reaction was stirred at 60° C. for 12 hours. The reaction was allowed to stir overnight, after which the reaction was shown to be about 60% conversion. After 24 hours, the reaction was poured over ice water and extracted with dichloromethane (3×50 mL), dried (sodium sulfate), filtered and concentrated to afford a tan solid. Purification by column chromatography (Luknova 40 g, 20 mL/min) using 10 to 100% ethyl acetate in hexanes over 65 minutes afforded 3-(hydroxy(2-(phenylsulfonyl)phenyl)methyl)-2,6,6-trimethyl-6,7-dihydro-1H-indol-4(5H)-one (153 mg, 0.361 mmol, 56% yield) as a white foam.

### Example 73B

### 2,6,6-trimethyl-3-(2-(phenylsulfonyl)benzyl)-6,7-dihydro-1H-indol-4(5H)-one

To a solution of 3-(hydroxy(2-(phenylsulfonyl)phenyl)methyl)-2,6,6-trimethyl-6,7-dihydro-1H-indol-4(5H)-one (158 mg, 0.373 mmol) in dichloromethane (10 ml) was added triethylsilane (119 μL, 0.746 mmol) followed by trifluoroacetic acid (86.0 μL, 1.12 mmol). Upon addition of trifluoroacetic acid, a gold reaction color was observed. The reaction was heated to 70° C. for 2 minutes, after which it was complete by LCMS. After cooling to room temperature, the reaction was quenched by the addition of saturated sodium bicarbonate solution (15 mL), then concentrated to remove the dichloromethane. A white precipitate formed, which was collected, washed with water, and dried to afford a white solid, 2,6,6-trimethyl-3-(2-(phenylsulfonyl)benzyl)-6,7-dihydro-1H-indol-4(5H)-one (142 mg, 0.348 mmol, 93% yield).

### Example 73C

### ethyl 2-(2,6,6-trimethyl-4-oxo-3-(2-(phenylsulfonyl)benzyl)-4,5,6,7-tetrahydro-1H-indol-1-yl)acetate (I-63)

To a vial was added 2,6,6-trimethyl-3-(2-(phenylsulfonyl)benzyl)-6,7-dihydro-1H-indol-4(5H)-one (140 mg, 0.344 mmol) potassium iodide (17.1 mg, 0.103 mmol), potassium carbonate (95.0 mg, 0.687 mmol), and acetonitrile (3 mL). Ethyl bromoacetate (0.191 ml, 1.72 mmol) was added, and the reaction was sealed and heated to 100° C. for 12 hours. Dilution of the reaction with water, followed by extraction with dichloromethane (3×30 mL), drying (sodium sulfate) and concentration afforded a gold residue. Purification was achieved by column chromatography on silica gel (Luknova 25 g, 20 mL/min) using 10 to 100% ethyl acetate in hexanes over 65 minutes. The product ethyl 2-(2,6,6-trimethyl-4-oxo-3-(2-(phenylsulfonyl)benzyl)-4,5,6,7-tetrahydro-1H-indol-1-yl)acetate (116 mg, 0.235 mmol, 68% yield) was isolated as an off-white solid. LCMS: [M+H]+ found, 494.2

The final step to the conversion of 2-(2,6,6-trimethyl-4-oxo-3-(2-(phenylsulfonyl)benzyl)-4,5,6,7-tetrahydro-1H-indol-1-yl)acetic acid as previously described.

### Example 74

### Synthesis of: 2-(3-(2-(cyclohexylsulfonyl)benzyl)-2,6,6-trimethyl-4-oxo-4,5,6,7-tetrahydro-1H-indol-1-yl)acetic acid (I-82)

### Example 74A

### Sodium Cyclohexanesulfinate

To a solution of sodium sulfite (4.66 g, 37.0 mmol) in water (20 mL) was added (portionwise) neat cyclohexanesulfonyl chloride (2.38 mL, 16.4 mmol) and solid sodium carbonate (3.10 g, 29.2 mmol). The reaction was heated to reflux for one hour then cooled to room temperature. The mixture was concentrated to dryness, then slurried in absolute ethanol. This slurry was heated to reflux for one hour, after which it was filtered and concentrated to afford sodium cyclohexanesulfinate (2.71 g, 15.9 mmol, 97% yield)

### Example 74B

### 2-(cyclohexylsulfonyl)benzaldehyde

A slurry of 2-fluorobenzaldehyde (1.02 mL, 9.79 mmol) and sodium cyclohexanesulfinate (2.00 g, 11.8 mmol) in N,N-dimethylformamide (40 mL) was heated to 110° C. for 12 hours. The reaction was cooled to room temperature then diluted in diethyl ether (50 mL) and extracted with diethyl ether (3×50 mL). The organic layer was dried (sodium sulfate), filtered, and concentrated. Purification by column chromatography (ISCO 80 g, 20 mL/min) using 5 to 75% ethyl acetate in hexanes afforded 2-(cyclohexylsulfonyl)benzaldehyde (748 mg, 2.96 mmol, 30% yield) as a white solid.

### Example 74C

### 3-((2-(cyclohexylsulfonyl)phenyl)(hydroxy)methyl)-2,6,6-trimethyl-6,7-dihydro-1H-indol-4(5H)-one

To a solution of 2,6,6-trimethyl-6,7-dihydro-1H-indol-4(5H)-one (525 mg, 2.96 mmol) and 2-(cyclohexylsulfonyl)benzaldehyde (748 mg, 2.96 mmol) in 2,2,2-trifluoroethanol (8 mL) was added a 3M aqueous solution of sodium hydroxide (2.96 mL, 8.89 mmol). The reaction was heated to 70° C. for 12 hours, then cooled to room temperature and diluted in water and diethyl ether (50 mL of each) and treated with excess sodium bisulfite. The biphasic solution was stirred at room temperature for 1.5 hours, after which the layers were separated and the organic layer was washed with water. The organic layer was concentrated, then reconstituted in hot methanol (heated until dissolved completely). Water was added, leading to the formation of a tan precipitate which was collected and dried to furnish 3-((2-(cyclohexylsulfonyl)phenyl)(hydroxy)methyl)-2,6,6-trimethyl-6,7-dihydro-1H-indol-4(5H)-one (691 mg, 1.61 mmol, 54% yield).

### Example 74D

### 3-(2-(cyclohexylsulfonyl)benzyl)-2,6,6-trimethyl-6,7-dihydro-1H-indol-4(5H)-one

To a slurry of 3-(2-(cyclohexylsulfonyl)phenyl)(hydroxy)methyl)-2,6,6-trimethyl-6,7-dihydro-1H-indol-4(5H)-one (695 mg, 1.62 mmol) in dichloromethane (15 mL) was added triethylsilane (0.517 ml, 3.24 mmol) followed by trifluoroacetic acid (0.374 ml, 4.85 mmol). Upon addition of trifluoroacetic acid, the slurry became an orange homogeneous solution. The reaction was stirred at room temperature for 30 minutes then heated at reflux for 5 minutes, cooled to room temperature, neutralized with 3N sodium hydroxide solution (125 μL), extracted with dichloromethane (3×40 mL), dried (sodium sulfate), filtered and concentrated to 3-(2-(cyclohexylsulfonyl)benzyl)-2,6,6-trimethyl-6,7-dihydro-1H-indol-4(5H)-one (719 mg, 1.74 mmol) as a tan solid. The reaction was used without purification in the next step.

### Example 74E

### ethyl 2-(3-(2-(cyclohexylsulfonyl)benzyl)-2,6,6-trimethyl-4-oxo-4,5,6,7-tetrahydro-1H-indol-1-yl)acetate (I-83)

To a solution of 3-(2-(cyclohexylsulfonyl)benzyl)-2,6,6-trimethyl-6,7-dihydro-1H-indol-4(5H)-one (710 mg, 1.72 mmol) in acetonitrile (4 mL) was added ethyl 2-bromoacetate (0.956 ml, 8.58 mmol), potassium iodide (114 mg, 0.687 mmol), and potassium carbonate (475 mg, 3.43 mmol). The reaction was heated to 110° C., after which 50% conversion was observed. The reaction was purified directly on silica gel (Luknova 120 g, 20 mL/min) to afford ethyl 2-(3-(2-(cyclohexylsulfonyl)benzyl)-2,6,6-trimethyl-4-oxo-4,5,6,7-tetrahydro-1H-indol-1-yl)acetate (241 mg, 0.482 mmol, 28% yield) as an off-white solid.

### Example 74F

### 2-(3-(2-(cyclohexylsulfonyl)benzyl)-2,6,6-trimethyl-4-oxo-4,5,6,7-tetrahydro-1H-indol-1-yl)acetic acid (I-82)

To a solution of ethyl 2-(3-(2-(cyclohexylsulfonyl)benzyl)-2,6,6-trimethyl-4-oxo-4,5,6,7-tetrahydro-1H-indol-1-yl)acetate (237 mg, 0.474 mmol) in THF (4 mL) and water (4.00 mL) was added a 3M aqueous solution of sodium hydroxide (0.316 ml, 0.949 mmol). After 30 minutes, there was roughly 40% conversion to the product. Another 4 equivalents of 3N sodium hydroxide solution was added (0.632 mL, 1.80 mmol). The reaction was complete after 20 minutes. The reaction was concentrated, then neutralized by the addition of 6N hydrochloric acid solution (0.475 mL). An off-white precipitate was observed, collected and dried to afford 24342-(cyclohexylsulfonyl)benzyl)-2,6,6-trimethyl-4-oxo-4,5,6,7-tetrahydro-1H-indol-1-yl)acetic acid (132 mg, 0.280 mmol, 59% yield). ¹H NMR (400 MHz, CDCl₃) δ (ppm): 7.95 (dd, 1H, J=8.0 Hz, J=1.6 Hz), 7.35 (app td, 1H, J=7.6 Hz, J=1.2 Hz), 7.25-7.30 (m, 1H), 6.96 (d, 1H, J=7.6 Hz), 4.59 (s, 2H), 4.51 (s, 2H), 3.45-3.52 (m, 1H), 2.56 (s, 2H), 2.28 (s, 2H), 2.04-2.06 (m, 5H), 1.85-1.89 (m, 2H), 1.54-1.70 (m, 3H), 1.17-1.40 (m, 3H), 1.11 (s, 6H).

### Example 75

### Synthesis of: 2-(2,6,6-trimethyl-4-oxo-3-(2-(pyrrolidin-1-ylsulfonyl)benzyl)-4,5,6,7-tetrahydro-1H-indol-1-yl)acetamide (I-84)

Methyl 2-(2,6,6-trimethyl-4-oxo-3-(2-(pyrrolidin-1-ylsulfonyl)benzyl)-4,5,6,7-tetrahydro-1H-indol-1-yl)acetate (246 mg, 0.521 mmol) was dissolved in 50 mL of ammonia (50 mL, 350 mmol) (7N) in an Ace Glass tube (sealed tube), and the resulting solution was charged with cyanopotassium (8.47 mg, 0.130 mmol). The tube was sealed and moved to a 80° C. oil bath, in which the reaction was stirred overnight. LC/MS showed about 95% starting material was gone after 18 hours. LC/MS 458.32 (M+1). The reaction was directly concentrated and the crude material was subjected to column chromatography (diluted with dichloromethane and filtered off cyanide salts prior to loading on column), using a 5-20% (ACN/MeOH 6:1) in dichloromethane as the gradient. 7.8 mg of 2-(2,6,6-trimethyl-4-oxo-3-(2-(pyrrolidin-1-ylsulfonyl)benzyl)-4,5,6,7-tetrahydro-1H-indol-1-yl)acetate was recovered, and 203.1 mg of 2-(2,6,6-trimethyl-4-oxo-3-(2-(pyrrolidin-1-ylsulfonyl)benzyl)-4,5,6,7-tetrahydro-1H-indol-1-yl)acetamide was isolated as a white powder. ¹HNMR (400 MHz, CDCl₃) 7.97 (dd, J=8.0, 1.6 Hz, 1H), 7.35-7.31 (m, 1H), 7.27-7.24 (m, 1H), 6.95 (d, J=7.2 Hz, 1H), 5.43 (br s, 1H), 4.57 (s, 2H), 4.48 (s, 2H), 3.39-3.36 (m, 4H), 2.61 (s, 2H), 2.32 (s, 2H), 2.02 (s, 3H), 1.96-1.92 (m, 4H), 1.14 (s, 6H); MS m/z: 458.32 (M+1).

### Example 76

### Synthesis of: 2-fluoro-2-(2,6,6-trimethyl-4-oxo-3-(2-(pyrrolidin-1-ylsulfonyl)benzyl)-4,5,6,7-tetrahydro-1H-indol-1-yl)acetic acid (I-92) and of Methyl 2-fluoro-2-(2,6,6-trimethyl-4-oxo-3-(2-(pyrrolidin-1-ylsulfonyl)benzyl)-4,5,6,7-tetrahydro-1H-indol-1-yl)acetate (I-93)

Methyl 2-(2,6,6-trimethyl-4-oxo-3-(2-(pyrrolidin-1-ylsulfonyl)benzyl)-4,5,6,7-tetrahydro-1H-indol-1-yl)acetate (0.130 g, 0.275 mmol) was dissolved in 3 mL THF and cooled to −78° C. LHMDS (0.110 g, 0.660 mmol) was added and the solution was stirred for 30 min. N-fluoro-N-(phenylsulfonyl)benzenesulfonamide (0.182 g, 0.578 mmol) was added and the reaction was warmed to room temperature and stirred overnight. At this time, the reaction was quenched with NH₄Cl and extracted with dichloromethane (3×20 mL). The organic portions were combined, dried (Na₂SO₄), filtered, and concentrated. The crude material was purified using SiO₂ chromatography (0-80% ethyl acetate in hexanes) to afford 56 mg of Methyl 2-fluoro-2-(2,6,6-trimethyl-4-oxo-3-(2-(pyrrolidin-1-ylsulfonyl)benzyl)-4,5,6,7-tetrahydro-1H-indol-1-yl)acetate as a white solid. ¹HNMR (400 MHz, CDCl₃) 7.98 (dd, J=7.6 Hz, 1.2 Hz, 1H), 7.34-7.23 (m, 2H), 6.88 (d, J=7.6 Hz, 1H), 6.30 (d, J=46.8 Hz, 1H), 5.29 (s, 2H), 4.50 (s, 2H), 3.90 (s, 1H), 3.38 (m, 4H), 2.60 (dd, J=16.4 Hz, 16.4 Hz, 2H), 2.28 (s, 2H), 2.06 (s, 3H), 1.90 (m, 4H), 1.11 (d, J=2.8 Hz, 6H).

Methyl 2-fluoro-2-(2,6,6-trimethyl-4-oxo-3-(2-(pyrrolidin-1-ylsulfonyl)benzyl)-4,5,6,7-tetrahydro-1H-indol-1-yl)acetate (0.056 g, 0.107 mmol) was dissolved in 1.5 ml of a 3:1:1 mixture of THF/MeOH/water and charged with lithium hydroxide (0.536 mmol). Reaction was stirred at rt for 1 h, at which time LC/MS suggest reaction is complete. Reaction was acidified with 1 N HCl, then extracted with dichloromethane (3×20 mL). The dichloromethane layers were combined, dried, filtered, and concentrated to yield 32 mg of 2-fluoro-2-(2,6,6-trimethyl-4-oxo-3-(2-(pyrrolidin-1-ylsulfonyl)benzyl)-4,5,6,7-tetrahydro-1H-indol-1-yl)acetic acid as a white solid. ¹HNMR (400 MHz, CDCl₃) 7.80 (dd, J=7.6 Hz, 1.2 Hz, 1H), 7.33-7.23 (m, 2H), 6.87 (d, J=7.6 Hz, 1H), 6.27 (d, J=46.8 Hz, 1H), 4.50 (s, 2H), 3.36 (m, 4H), 2.73 (dd, J=16.4, 16.4, 2H), 2.34 (s, 2H), 2.05 (s, 3H), 1.90 (m, 4H), 1.01 (s, 6H).

### I-94

### Example 77

### Example 77

### Synthesis of: 2-(2,5,5,6,6-pentamethyl-4-oxo-3-(2-(pyrrolidin-1-ylsulfonyl)benzyl)-4,5,6,7-tetrahydro-1H-indol-1-yl)acetic acid (I-95)

Step 1 Boc Protection

2,6,6-trimethyl-3-(2-(pyrrolidin-1-ylsulfonyl)benzyl)-6,7-dihydro-1H-indol-4(5H)-one (3.0 g, 7.49 mmol) was diluted with benzene (60 ml) and consecutively charged with di-tert-butyl dicarbonate (3.27 g, 14.98 mmol) and DMAP (0.137 g, 1.123 mmol). Flask was then fitted with a reflux condenser and the system was heated to reflux and stirred for 4 hours. At this point, TLC suggests reaction is complete (product spot ˜R_{f}=0.7 using 1:1 Hexane/Ethyl acetate). Reaction is then quenched with sodium bicarbonate solution. Heterogeneous mixture is moved to a separatory funnel and the layers were separated. The aqueous layer was then extracted with an additional 50 mL DCM. The combined organic portions were dried (Na₂SO₄), filtered, and concentrated. The crude oil (tert-butyl 2,6,6-trimethyl-4-oxo-3-(2-(pyrrolidin-1-ylsulfonyl)benzyl)-4,5,6,7-tetrahydro-1H-indole-1-carboxylate) was purified using SiO2 chromatography—gradient elution—0-60% Ethyl acetate in hexanes yielding 3.15 g of tert-butyl 2,6,6-trimethyl-4-oxo-3-(2-(pyrrolidin-1-ylsulfonyl)benzyl)-4,5,6,7-tetrahydro-1H-indole-1-carboxylate.

Step 2 Methylation (bis)

(step 1) tert-butyl 2,6,6-trimethyl-4-oxo-3-(2-(pyrrolidin-1-ylsulfonyl)benzyl)-4,5,6,7-tetrahydro-1H-indole-1-carboxylate (0.200 g, 0.399 mmol) was dissolved in THF (5 ml), cooled to −78° C. and was then charged with LHMDS (1.099 ml, 1.099 mmol). Reaction was stirred at this temperature for 1 hour—over time, r×n turned yellow. After 1 hour, methyl iodide (0.052 ml, 0.839 mmol) was added and the reaction was slowly warmed to room temperature and stirred at that temperature overnight. In the morning, the reaction was quenched with NH₄Cl and extracted 2× ethyl acetate. The combined organic layers were dried (Na₂SO₄), filtered, and concentrated. The crude oil was purified using SiO2 chromatography—gradient elution—0-60% ethyl acetate in hexanes to afford 191 mg of tert-butyl 2,5,6,6-tetramethyl-4-oxo-3-(2-(pyrrolidin-1-ylsulfonyl)benzyl)-4,5,6,7-tetrahydro-1H-indole-1-carboxylate.

(step 2) tert-butyl 2,5,6,6-tetramethyl-4-oxo-3-(2-(pyrrolidin-1-ylsulfonyl)benzyl)-4,5,6,7-tetrahydro-1H-indole-1-carboxylate (0.107 g, 0.208 mmol) was subjected to conditions laid forth in the previous step to afford 76 mg of tert-butyl 2,5,5,6,6-pentamethyl-4-oxo-3-(2-(pyrrolidin-1-ylsulfonyl)benzyl)-4,5,6,7-tetrahydro-1H-indole-1-carboxylate

Step 3 Boc-Deprotection

tert-butyl 2,5,5,6,6-pentamethyl-4-oxo-3-(2-(pyrrolidin-1-ylsulfonyl)benzyl)-4,5,6,7-tetrahydro-1H-indole-1-carboxylate (0.076 g, 0.144 mmol) was dissolved in dichloromethane (4 ml) and directly charged with TFA (1.0 ml, 12.98 mmol). The reaction was heated to 50° C. and stirred for 2 hours. At this time the reaction was concentrated and the crude was taken up in 20 ml of dichloromethane and washed with NaHCO₃. The organic was then isolated, dried (Na₂SO₄), filtered, and concentrated. The crude material 2,5,5,6,6-Pentamethyl-3-(2-(pyrrolidin-1-ylsulfonyl)benzyl)-6,7-dihydro-1H-indol-4(5H)-one was taken directly on to alkylation without further purification.

Step 4 Alkylation

2,5,5,6,6-Pentamethyl-3-(2-(pyrrolidin-1-ylsulfonyl)benzyl)-6,7-dihydro-1H-indol-4(5H)-one (0.065 g, 0.152 mmol) was dissolved in 2 mL of acetonitrile and charged with potassium carbonate (0.063 g, 0.455 mmol). Ethyl bromoacetate (0.034 ml, 0.303 mmol) was then added in a dropwise fashion. Reaction was moved to an 85° C. hot plate and stirred for 14 h. At this time, it was cooled and quenched with NH₄Cl. The quenched reaction was extracted with dichloromethane (3×) and the combined organic portions were dried (Na₂SO₄), filtered, and concentrated. The crude material was purified using a 0-70% ethyl acetate in hexanes gradient to afford 39 mg of Ethyl 2-(2,5,5,6,6-pentamethyl-4-oxo-3-(2-(pyrrolidin-1-ylsulfonyl)benzyl)-4,5,6,7-tetrahydro-1H-indol-1-yl)acetate.

Step 5 Saponification

Ethyl 2-(2,5,5,6,6-pentamethyl-4-oxo-3-(2-(pyrrolidin-1-ylsulfonyl)benzyl)-4,5,6,7-tetrahydro-1H-indol-1-yl)acetate (0.039 g, 0.076 mmol) was subjected to conditions laid forth in previous examples to afford 30 mg of 2-(2,5,5,6,6-pentamethyl-4-oxo-3-(2-(pyrrolidin-1-ylsulfonyl)benzyl)-4,5,6,7-tetrahydro-1H-indol-1-yl)acetic acid ¹HNMR (400 MHz, CDCl₃) 8.97 (bs, 1H), 7.97 (dd, J=7.6, 1.2 Hz, 1H), 7.30-7.18 (m, 2H), 6.99-6.92 (m, 1H), 4.54 (s, 4H), 3.37-3.34 (m, 4H), 1.96 (s, 3H), 1.91-1.89 (m, 4H), 1.07 (s, 12H).

### Example 78

### Synthesis of: 2-(5,5-difluoro-2,6,6-trimethyl-4-oxo-3-(2-(pyrrolidin-1-ylsulfonyl)benzyl)-4,5,6,7-tetrahydro-1H-indol-1-yl)acetic acid (I-96)

2-(5,5-difluoro-2,6,6-trimethyl-4-oxo-3-(2-(pyrrolidin-1-ylsulfonyl)benzyl)-4,5,6,7-tetrahydro-1H-indol-1-yl)acetic acid was synthesized according to the procedure described above for 2-(2,5,5,6,6-pentamethyl-4-oxo-3-(2-(pyrrolidin-1-ylsulfonyl)benzyl)-4,5,6,7-tetrahydro-1H-indol-1-yl)acetic acid with the following exception: N-fluoro-N-(phenylsulfonyl)benzenesulfonamide was used as the electrophile instead of methyl iodide in the second step. ¹HNMR (400 MHz, CDCl₃) 9.35 (bs, 1H), 7.97 (dd, J=7.6 Hz, 1.2 Hz, 1H), 7.32-7.22 (m, 2H), 6.88 (d, J=7.2 Hz, 1H), 4.57 (s, 2H), 4.51 (s, 2H), 3.39-3.35 (m, 4H), 2.75 (s, 3H), 1.95 (s, 3H), 1.94-1.91 (m, 4H), 1.21 (s, 6H).

### Example 79

### Synthesis of: 2-(5,5-difluoro-2,6,6-trimethyl-3-(2-(pyrrolidin-1-ylsulfonyl)benzyl)-4,5,6,7-tetrahydro-1H-indol-1-yl)acetic acid (I-97)

2-(5,5-difluoro-2,6,6-trimethyl-4-oxo-3-(2-(pyrrolidin-1-ylsulfonyl)benzyl)-4,5,6,7-tetrahydro-1H-indol-1-yl)acetic acid (0.100 g, 0.202 mmol) was dissolved in 5 mL THF, cooled to 0° and directly charged with LAH. Reaction was monitored closely by LC/MS. Upon completion, reaction was carefully quenched with NH₄Cl and moved to a separatory funnel. The layers were separated and the aqueous portion was extracted 2 additional times with dichloromethane. The organic layers were then combined, dried (Na₂SO₄), and concentrated. The crude material was immediately taken up in 5 mL dichloromethane and cooled to 0° C. 2,2,2-trifluoroacetic acid (0.322 mmol) was then added followed by triethylsilane (0.644 mmol). After 5 minutes, reaction was concentrated and purified on reverse-phase HPLC to yield 26 mg of 2-(5,5-difluoro-2,6,6-trimethyl-3-(2-(pyrrolidin-1-ylsulfonyl)benzyl)-4,5,6,7-tetrahydro-1H-indol-1-yl)acetic acid. ¹HNMR (400 MHz, CDCl₃) 7.95 (d, J=7.6 Hz, 1H), 7.40-7.26 (m, 2H), 7.02 (d, J=7.6 Hz, 1H), 6.5 (bs, 1H), 4.53 (s, 2H), 4.14 (s, 2H), 3.37-3.33 (m, 4H), 2.63 (m, 2H), 2.50 (s, 32), 2.04 (s, 3H), 1.95-1.92 (m, 4H), 1.11 (s, 6H).

The following compounds exemplify further embodiments of the invention disclosed herein.

Compounds in which A is an eight, nine or ten-membered carbocycle may be made in analogous fashion from commercially available (Sigma Aldrich) starting materials: cyclooctanone, cyclononanone and cyclodecanone.

Animal Models Related to Allergic Response

Any of a variety of animal models and in vitro assays can be used to test the compounds for their effectiveness in reducing allergic and inflammatory activity. Useful compounds can exhibit effectiveness in reducing allergic response and inflammation in one or more animal models or in vitro assays.

Induction of Contact Hypersensitivity. In this model, induction of contact hypersensitivity (CHS) is created as described by Takeshita et al. (2004. Int. Immunol. 16(7):947-59). On days 0 and 1, female Balb/c mice, 7-8 weeks of age are painted onto the shaved abdominal skin with 400 μl of 0.5% fluorescein isothiocyanate (FITC) dissolved in acetone:dibutylphthalate (1:1, DBP). Six days later, mice are challenged by application of 20 μl of 0.5% FITC in DBP onto both sides of the right ear. The solvent control (DBP) is applied to the left ear. Challenge-induced increases in ear thickness are measured by an engineer's micrometer at 0, 24, 48 and 72 hours post-challenge. The CHS response is determined by challenge-induced increases in ear thickness. CHS response=[(right ear thickness post challenge−left ear thickness post challenge)−(right ear thickness pre challenge−left ear thickness pre challenge)].

To determine the presence of leukocyte infiltration, ears and back skins are fixed for 30 hours in zinc fixative at room temperature and embedded in paraffin for histological and immunohistochemical evaluation. For assessment of eosinophil peroxidase activity (EPO), skin sections are homogenized in 1 ml of ice cold buffer (0.05 M Tris-HCl pH 8.0 containing 0.1% Triton X-100). The tissue samples are centrifuged at 10,000 g for 20 minutes at 4° C. and supernatants are collected for measurement of EPO activity. In a 96 well microtiter plate, the substrate solution (100 μl of 10 mM o-phenylenediamine in 0.05 M Tris-HCl and 4 mM H₂O₂) is added to the 20-fold diluted homogenate in buffer (100 μl). The reaction mixture is incubated at room temperature for 1 hour before the reaction is stopped by the addition of 100 μl of 2M sulfuric acid. The microtiter plate is measured for absorbance.

Evan's Blue Test. Complete protocol details can be found in Takeshita et al. (2004. Int. Immunol. 16(7):947-59). Briefly, female Balb/c mice, 7 weeks of age are injected at two locations intradermally on their shaved backs with increasing concentrations of 0.1-10 μg/site of DK-PGD₂. This is followed by an intravenous injection of 0.25 ml of saline containing 1.25 mg of Evan's blue dye. Four hours post-dye injection, mice are euthanized and the back skin is collected. Edema severity is assessed by measuring the density of the extravasated dye. Effects of pharmacological inhibition of the inflammatory reaction to DK-PGD₂ will also be assessed by treatment with CRTH2 antagonists, such as Ramatroban.

Ovalabumin-Induced Airway Cell Proliferation and Inflammation. Complete protocol details can be found in Eynott et al. (2003. J. Pharmacol. Ther. 304:22-29). Briefly, Brown Norway rats are sensitized on days 1, 2, and 3 with intraperitoneal (i.p.) injections of 1 mg ovalbumin (OVA) and 100 mg Al(OH)₃ in 1 mL 0.9% NaCl saline. They are then exposed to either 0.9% NaCl saline or 1% OVA aerosol every 3rd day (days 6, 9, & 12) for 30 minutes. 2 mg/kg dexamethasone is used as a positive control and is dosed i.p. once a day on days 4, 5, 6, 9, & 12. Vehicle (15% β-cyclodextrins in DMSO) and test compounds are dosed orally twice a day on days 5-12. On challenge days, all animals are treated 1 hour prior to OVA allergen exposure and, if required for twice a day treatment, ˜4-8 hours after allergen exposure. Samples are collected 24 hours after the last OVA challenge. For sample collection, rats are anaesthetized by administration of 10 mg/kg xylazine and 60 mg/kg ketamine intraperitoneally. Once the rats were fully anesthetized, blood is collected for serum via the retro-orbital route. The rats are subsequently perfused by injecting 30 mL PBS through the right ventricle of the heart after the abdominal aorta is severed. A tracheotomy is then performed and bronchioalveolar lavage fluid (BAL) is collected through five 5 mL rinses using Hank's Balanced Salt Solution, which was kept on ice. Airway inflammatory cell accumulation and proliferation of cells are measured through the BAL fluid collection and subsequent cell counts. Cytospin slides are prepared and eosinophil % are determined by counting ˜400 cells per slide. The test compounds are dosed at 5 mg/kg twice daily at various concentrations. Activity is scored based on the ability of the test compound to prevent ovalbumin-induced eosinophil induction (as determined by percentage of eosinophils in BAL fluid).

Ovalbumin-induced Airway Inflammation in Sensitized Brown Norway Rats. The assay assesses the effect of test compounds on cellular recruitment into the lung after antigen challenge in the sensitised Brown Norway rat. The model is a slightly modified protocol based on that disclosed in Underwood et al. 2002 British Journal of Pharmacology 137: 263-275. Briefly, male Brown Norway rats (200-225 g, from Harlan) are be sensitised on days 0, 14 and 21 with ovalbumin (100 μg/rat, i.p.) administered with Alum™ (20 mg/rat aluminium hydroxide and 20 mg/rat magnesium hydroxide, i.p.). Rats are challenged with inhaled ovalbumin (10 g/l, 30 minutes) or saline aerosol on day 28. Vehicle (5 ml/kg) or test compound (1 or 10 mg/kg, 5 ml/kg) are dosed orally 16 and 1 hour(s) before and 1 and 6 hours after antigen challenge. Budesonide (3 mg/kg) is included as a positive control and dosed at the same time points. End point measurements are as follows; one hour after the challenge the rats have Enhanced Pause (PenH) levels monitored for 5 hours to assess late asthmatic reaction.

Cellular burden and inflammatory status are assessed. Twenty-four hours after ovalbumin challenge, rats are euthanised with an overdose of pentobarbitone i.p. A heparinised blood sample is taken via cardiac puncture and the resulting plasma kept frozen. Bronchioalveolar lavage (BAL) is carried out (2×3 ml RPMI media, 30 seconds each). Immediately after BAL, the left lobe is removed, perfused with RPMI to remove the blood pool of cells and 300 mg of lung is chopped and stored in RPMI/FCS (fetal calf serum) containing penicillin/streptomycin. The remaining perfused, chopped lung tissue is flash frozen and stored at −80° C. The remaining lung lobes are insufflated with formalin to a pressure of 20 mmHg, the lungs tied off and stored in formalin until required.

The 300 mg of tissue undergoes collagenase digestion and the cells are recovered (For method see Underwood et al., (1997) Br. J. Pharm., 122, 439-446). Total cell counts recovered from the airway lumen and lung tissue are quantified using a Sysmex cell counter. Differential cell counts (200 cells counted which comprise eosinophils, neutrophils, lymphomononuclear cells expressed as percentage and absolute cell counts) of cells recovered from the airway lumen and lung tissue are made by light microscopy from cytocentrifuge preparations stained with Wright-Giemsa stain. Remaining BAL samples are spun down and supernatant retained at −20° C. Additionally, this model can be used to assess the effect of agents described herein on airway resistance.

Sephadex induced-Pulmonary Eosinophilia in Rodents. Male Swiss Webster mice are used in a model of Sephadex induced-Pulmonary Eosinophilia. In brief, test groups receive vehicle, test compound (10 mg/kg) or positive control, dexamethasone (0.5 mg/kg), by oral gavage, twice per day (p.o., b.i.d.) at a dosing volume of 10 ml/kg, on days −1, 0, 1 and once, 4 hours pre-sacrifice, on day 2. On day 0, test groups are each intravenously administered 3 mg/kg Sephadex beads G-100-120 (Sigma) at a dosing volume of 5 ml/kg or no Sephadex. On day 2, four hours post vehicle/test compound/dexmethasone administration, animals are euthanized by inhalation of CO₂ and subsequently undergo histopathologic and lavage evaluation of lungs for severity of eosinophilic infiltrate in peribronchiolar locations. Bronchioalveolar lavage fluid is collected by flushing the lung via the trachea 3 times with 1 ml aliquots of cold saline, and then the lungs are harvested by filling with formalin and allowed fixation a minimum of 1 day. White blood cell counts are prepared from lavage fluids. In addition, lavage fluids are immediately prepared for cytospin and cell differential counts performed. Cytospin slides are stained with a Wrights-Giemsa stain. Whole lung sections are stained with Hematoxylin and eosin stain for morphometry evaluation of severity of inflammatory cell infiltrate in peribronchiolar locations around Sephadex beads. Three sections (initial and 2 steps at 100 μm intervals) are prepared from each animal for analysis of area or diameter of inflammation around 5-8 Sephadex beads/mouse. Morphometric digital imaging analysis is performed to score inflammation. A similar experimental protocol can be performed using Lewis rats with the modification that animals are euthanized on day 1.

Mouse Model of Allergic Airways Disease Using the FlexiVent System. In this model, animals in groups of 10 (8-10 wk old male BALB/c mice) are used to assess allergic airway disease. Mice are quarantined for 14 days. On days 0 (the first day following the end of the 14 day quarantine) and day 7, experimental animals are immunized by intraperitoneal (i.p.) injection with a mixture of ovalbumin (OVA; 10 μg) and aluminum hydroxide (Alum; 2 mg) in sterile water. A second group of animals is immunized with sterile water only and serves as a nonimmunized (negative) control. On days 13, 14, 15, and 16, dexamethasone (positive control), test compound or vehicle only is delivered by oral gavage (all at 10 mg/kg and a dosing volume of 10 ml/kg) twice a day. Animals are exposed to ovalbumin on days 14 and 15. Ovalbumin exposures are generated by aerosolizing 1% heat-aggregated ovalbumin (chicken egg, grade V; Sigma, St. Louis, Mo.), diluted with filtered air, and then delivered to the exposure chambers for 3 hours (H2000, Hazelton Systems). The total mass concentration of ovalbumin is determined by gravimetric analysis of filter samples taken every hour during exposure. The target mass concentration of ovalbumin is 4 mg/m³. Chamber temperatures are maintained at 26±2° C. and lights on a 12 hour on/off cycle. Animals are given food (Teklad™ certified rodent diet (Harlan Teklad, Madison, Wis.)), ad libitum except during the 3 hour exposure period. Water is available ad libitum throughout the duration of the study.

On day 17, animals are anesthetized and tested for pulmonary function (response to methacholine challenge) by forced oscillation techniques (FlexiVent). Airway hyperresponsiveness (AHR) to increasing concentrations of aerosolized methacholine (MCh) is measured using a FlexiVent analyzer (SCIREQ, Montreal, Canada). Briefly, each mouse is anesthetized with Avertin (250 mg/kg; 0.02 ml/g; 1.2% (w/v) solution of 2,2,2 tribromoethanol in 0.8% tert-amyl ethanol (2 methyl, 2 butanol)) i.p. and placed on a heating pad. The neck fur is shaved and a small superficial incision made in the skin above the trachea. After the lobes of the salivary gland are separated, a small incision is made in the trachea, and the trachea is cannulated with a blunt-end 20 gauge needle hub. The cannula is secured by suture thread and the skin is pulled back and secured by cyanoacrylate adhesive. Ventilation is performed through the cannula by positive pressure maneuvers on the Flexivent apparatus. Once on the ventilator, pancuronium, (paralytic, 0.5 mg/kg) is administered i.p. Heart rate is monitored via a Grass Instruments Recorder w/Tachograph. Changes in heart rate greater than 50 bpm from baseline require supplementing the anesthesia (Avertin, ip). Additional doses of Avertin are given at a dose of 100 mg/kg and the animal's heart rate is monitored for at least 60 sec to determine if additional doses are needed. After baseline measurements of resistance and compliance, increasing doses of methacholine (Mch; 3, 6, 12, 25, 50 mg/ml nebulizer) are delivered via aerosol and resistance and compliance are measured. Airway resistance is calculated for each concentration of methacholine and the average ±SEM is plotted for all treatment groups. Changes in pulmonary resistance (i.e., Mch dose-response curves) are assessed by repeated measures two way analysis of variance (ANOVA) with Bonferroni post-test. All other statistical comparisons are made using ANOVA with the Dunnetts multiple comparison test. A value of p<0.05 is considered significant.

Following AHR measurements, blood is collected and saved for further evaluation. The animals are then euthanized by injection with a lethal dose of a pentobarbital-based euthanasia solution. Bronchioalveolar lavage (BAL) cells are obtained from 7 animals per experimental or control group by inserting a catheter into the trachea and lavaging the lung 3 times with 0.8 ml of PBS (without calcium chloride and magnesium chloride). Total BAL cells are determined using a hemacytometer. BAL cells are spun onto slides by cytocentrifugation and stained with a modified Wright-Giemsa stain. Four hundred cells are counted and the percentage of specific cell types determined for each animal. The first lavage fluid sample (after centrifugation) is frozen separately for future cytokine analysis. The whole lung is snap frozen dry for future analyses.

Three animals from each group which are not subjected to BAL are used for histopathologic analysis and have their lungs instilled via the trachea with 10% buffered formalin, removed and fixed in the same solution. Generally, three specimens per treatment, each consisting of multiple axial sections of lung, are examined. All sections are stained with alcian blue-H&E. Lesions are graded on a subjective basis. Lesions are graded as minimal, mild, moderate, and marked (corresponding to severity scores of 1, 2, 3, and 4, respectively) and given a distribution designation of either focal, locally extensive, multifocal, multifocal and coalescing, or diffuse (corresponding to distribution scores of 1, 2, 3, 4 and 5, respectively). The product of the severity and distribution scores is averaged for each treatment group.

Prostaglandin D₂-induced Eosinophilic Airway Inflammation. Complete protocol details can be found in Shiraishi et al (2004. J. Pharmacol. Ther. epub as DOI:10:1124/jpet.104.078212). Briefly, Brown Norway rats are intravenously injected with rat interleukin-5 or PBS, one hour prior to intratracheal administration of prostanoid receptor agonists. These agonists can include the following; PGD₂, two CRTH₂-specific agonists, DK-PGD₂, 15R-methyl PGD₂, and 11-deoxy-11-methylene-15-keto-PGD₂ (MK-PGD₂), a DP receptor-specific agonist BW 245C, a thromboxane A₂ receptor (TP)-specific agonist, -BOP and Indomethacin. In some experiments, an orally delivered CRTH2/TP antagonist, Ramatroban, an intravenously delivered DP antagonist, BW A868C, or an intravenously delivered TP antagonist are administered two hours prior to administration of agonists. Rats are euthanized at 2, 8 and 24 hours post-agonist administration. Inflammatory cell accumulation in the trachea and lungs is recovered by bronchioalveolar lavage for cell counts and lungs are evaluated by histological examination. In a separate experiment, rats receive intravenous injection of IL-5 (0.2 ng/kg) or PBS one hour prior to intratracheal administration of PGD₂ (100 nmoles/animal) or vehicle. A peripheral blood sample is collected hourly post-dose of IL-5 for hematological evaluation.

Murine Allergic Inflammation. Complete protocol details are described in Fujitani et al. (2002 J. Immunol 168:443-449) and Matsuoka et al. (2000. Science 287: 2013-2017). Briefly, transgenic and wildtype mice are immunized with 10 μg ovalbumin (OVA) in 0.2 ml aluminum hydroxide (Alum) on days 0 and 14. On day 21, the mice are exposed to aerosolized OVA (50 mg/ml in sterile saline) for 20 minutes. On days 1 and 3 post-OVA challenge, mice are euthanized, bronchioalveolar lavaged, and the lavage fluid is assessed by differential cell counting.

Allergic Rhinitis in Anesthetized Rodents. In this model described, for example, by Arimura et al. (2001 J. Pharmacol. Ther. 298:411-419) guinea pigs are sensitized to OVA twice by inhalation of an aerosol solution of 1% OVA for 10 minutes. At 7 days after the second sensitization, the animals are anesthetized and artificially ventilated through a tracheal cannula using a respirator. Another glass cannula is inserted into the nasopharynx from the side of the larynx, and a fixed amount of air is continuously insufflated into the nasal cavity via the nasal cannula using another respirator. Insufflation pressure is monitored by a pressure transducer connected to the side arm of the nasal cannula as an indication of intranasal pressure. Nasal antigen challenge is performed by generating an aerosol of 3% OVA between the nasal cannula and the animal respirator for 3 minutes using an ultrasonic nebulizer, and then the intranasal pressure is measured for 30 minutes. Nasal secretion and the nose are collected for further evaluation.

A biphasic allergic rhinitis model in conscious guinea pigs is also fully described in Arimura et al. (2001 J. Pharmacol. Ther. 298:411-419).

Allergic Conjunctivitis Model. Complete protocol details are described in Arimura et al. (2001 J. Pharmacol. Ther. 298:411-419). Briefly, a 2.5% OVA solution is applied topically to both eyes (10 μl/eye) of conscious guinea pigs that have been sensitized as described in the “Allergic Rhinitis Model in Anesthetized Rodents” protocol above. Immediately following OVA application, Evan's blue dye (20 mg/kg i.v.) is injected as a marker of plasma exudation. The amount of Evan's blue extravasated in the conjunctiva and eyelid for 30 minutes is quantified. Independently, histamine 0.001%, PGD₂ 0.01%, or a combination of the two are applied to the eyes of nonsensitized guinea pigs, and dye exudation is determined.

Determination of Interleukin-13 Levels in Bronchial Alveolar Lavage Fluid. A commercially available ELISA kit (Biosource, Catalog #KRC0132) is used to determine the effects of compounds on the Interleukin-13 (IL-13) levels of bronchial alveolar lavage fluid (BALF) taken from rats that have undergone certain allergen induced (e.g. ovalbumin, sephadex, prostaglandin D₂) airway cell proliferation and inflammation.

After collection, BALF samples are concentrated 5-fold with Microcon YM-3 centrifugal devices (Millipore, Catalog #42404) and stored at −80° C. until use. A 500 pg/mL standard stock is prepared by reconstituting the IL-13 standard provided in the kit with the amount of standard diluent specified on the standard vial. A standard curve is then prepared by serially the standard stock down to 7.8 pg/mL. 50 μL of each point of the standard curve and 50 μL of concentrated BALF sample are added to the ELISA plate. Added to these samples is 150 μL of anti-rat IL-13 biotin conjugate. The plate is then incubated at room temperature for 2 hours. The plate is then washed 4 times with wash buffer and 100 μL of 1-x streptavidin-peroxidase is added to all wells. The samples are then incubated at room temperature for 30 minutes. Again, the plate is washed 4 times with wash buffer. 100 μL of stabilized chromogen are added to each well and the plate is incubated at room temperature for 45 minutes. To stop the reaction, 100 μL of stop solution is added and the plate is read at 450 nm. Levels of other cytokines including IL-1β, IL-4, IL-5 and the chemokine, eotaxin can be similarly assessed in BALF samples to determine the effect of test compounds on Th-2 related function.

Determination of Ovalbumin specific Immunoglobulin E in Serum. The effects of compounds on serum immunoglobulin E (IgE) levels in rodents that have undergone allergen-induced (e.g. ovalbumin) airway cell proliferation and inflammation can be measured using an assay developed with reference to Salgado et al., Allergol. et Immunopathol., 16, 2 (95-98), 1988.

Serum samples are taken from rats suffering from asthma, induced by the inhalation of ovalbumin, and stored at −80° C. until use. The ELISA plate is coated with 1.25 mg/mL ovalbumin prepared in coating buffer (0.5M Carbonate-Bicarbonate, pH 9.6, Bethyl Labs, Catalog #E107) and incubated overnight at 4° C. After 18 hours, the plate is washed one time with wash buffer (50 mM Tris, 0.14 M NaCl, 0.05% Tween 20, pH 8.0, Bethyl Labs, Catalog #E106). 200 μL of blocking solution (5% skim milk/PBS) is added and the plate is incubated at 4° C. for 1 hour. Serum samples are diluted 1:3000 in sample diluent (Post coat solution containing 50 mM Tris, 1% BSA, pH 8.0 0.05% Tween 20, Bethyl Labs, Catalog #E104). After the one hour incubation with blocking solution, the plate is washed three times with wash solution and 100 μL of diluted sample is added to the appropriate well. Samples are then incubated at room temperature for 3 hours. Once the 3 hour incubation is complete, the plate is washed five times with wash buffer. The sheep anti-rat IgE HRP conjugate detection antibody (Bethyl Labs, Catalog #A110-117P) is diluted 1:100 in a 1% skim milk/PBS solution. 100 μL of this solution is then added to the plate and the plate is incubated for 1 hour at 4° C. The plate is then washed another five times with wash buffer. The TMB peroxidase substrate (Bethyl Labs, Catalog #E102) is prepared by adding equal volumes of TMB peroxidase substrate with Peroxidase solution B. 100 μL of substrate is added to plate and incubated at room temperature for 15 minutes. The enzymatic reaction is stopped by adding 100 μL of 2 M sulfuric acid (Sigma Aldrich). The plate is then read at a wavelength of 450 nm.

Determination of Methacholine responsiveness in mice 2-8 weeks of age Complete protocol details for this model can be found in Bozanich et al. (J Appl Physiol 103: 542-546, 2007). Briefly, the animals are prepared, anaesthetized, tracheotimized, connected to a ventilator, and cannulated as described. Two small electrodes are placed into the intercostal muscles of the mouse and connected to an electrical stimulator (Grass Instruments, Quincy, Mass.). Ventilation is paused, the positive end-expiratory pressure is removed with the airway, and the plethysmograph is opened to atmosphere to allow the lungs to reach the elastic equilibrium volume at transrespiratory pressure of 0 hPa, defined as functional residual capacity (FRC). With the plethysmograph closed and the airway occluded, five to eight stimulated breathing efforts are induced over a 10 sec period. FRC is then calculated using Boyle's principle. Lung volume (VL) is then increased by lowering the plethysmograph pressure from 0 to −20 hPa in a quasi-linear fashion during 15-20 sec. The increase in VL from FRC to transrespiratory pressure=20 hPa (VL20) achieved during the slow deep inflation (sDI) maneuver is determined by integrating the flow into the animal through the wave tube as described. The inflation phase is followed by a slow passive expiration to transrespiratory pressure ═O hPa, where the measurement of FRC is repeated in a subgroup of animals. Respiratory system impedance (Zrs) is measured using a low-frequency (4-38 Hz) forced oscillation technique and a wave-tube system as described. Doubling doses (6-48 ug/min-kg) of beta-methacholine chloride (MCh; Sigma-Aldrich) are delivered for 5 min by constant infusion via the jugular vein cannula. A steady-state constriction is achieved by 5 min and is verified by monitoring tracheal pressure during mechanical ventilation. FRC is measured, and a single slow deep inhalation (sDI) maneuver is performed with the infusion continuing to run. Test compound or vehicle alone is administered, for example, orally, twice daily for 1-4 days prior to receiving the MCh treatment and may also include dosing approximately 4 hours before MCh treatment. Differences in FRC before and after an sDI maneuver performed at baseline and the maximum MCh dose in a mice subgroups (for example, 3-10 mice from each age group) are determined using paired t-tests. MCh responsiveness in the presence and absence of test compound is calculated as described for each animal group.

Murine Model of Atopic Dermatitis. This model is described, for example, by Spergel et. al. (1998 J. Clin. Invest. 101: 1614-1622). Epicutaneous (EC) sensitization of mice was performed as described by Wang et al. (1996 J. Immunol. 156:4079-4082). Briefly, 4-6 week old BALB/c mice were anesthetized with methoxyflurane (Metofane; Mallinckrodt Veterinary, Mundelein, Ill.), then shaved with an electric razor. 100 μg of OVA (grade V; Sigma Chemical Co., St. Louis, Mo.) in 100 μl of normal saline or placebo (100 μl of normal saline) was placed on a 1×1 cm patch of sterile gauze, which was secured to the skin with a transparent bioocclusive dressing (Johnson and Johnson Medical Inc., Arlington, Tex.). The patch was placed for a 1-wk period and then removed. 2 wk later, an identical patch was reapplied to the same skin site. Each mouse had a total of three 1-wk exposures to the patch separated from each other by 2-wk intervals. Inspection confirmed that the patch remained in place at the end of each sensitization period. For a positive control, intraperitoneal (IP) sensitization of another group of mice was performed with OVA (100 μg)-alum and boosted 2 wk later with the same dose of OVA in alum.

Mice are bled and sera collected 1 hour following the end of the series of three EC sensitizations by the standard PharMingen ELISA protocol used to quantify the total amount of IgE in serum. OVA specific antibodies in the serum can also be assessed, as well as cellular infiltrate into the skin by histological and immunohistochemical analysis. Also, the presence of mRNA for cytokines in skin sites sensitized with OVA can be detected via RT-PCR (protocol details are fully described in Spergel et. al., 1998 J. Clin. Invest. 101: 1614-162).

BAL fluid can also be examined in this model. EC sensitized mice are challenged with a single exposure to inhaled 1% OVA via a nebulizer for 20 minutes, and 24 hours later BAL fluid is examined for the presence of eosinophils and other cellular infiltrate (protocol details are fully described in Spergel et. al., 1998 J. Clin. Invest. 101: 1614-162).

Airway hyperresponsiveness can also be assessed in this model described by Spergel et. al., 1996. Briefly, 24 hours after one dose of nebulized 1% OVA, airway measurements are measured plethysmographically in sedated, ventilated mice in response to graded doses of intravenous methacholine.

DK-PGD2-Induced Systemic Eosinophilia in Rats. Female Sprague-Dawley rats (175-250 g) were dosed orally with test compound (or vehicle). Thirty minutes after dosing, animals were anaesthetized with isoflurane. Following induction of anaesthesia, animals received an intracardiac injection of 10 μg DK-PGD2 in 0.3 ml heparinized (10 U/ml) saline. Control animals received an injection of 0.3 ml heparinized saline. Sixty minutes after the intracardiac injection, animals were again anesthetized with isoflurane and a blood sample was drawn from the abdominal aorta (into heparin) while the rat was anaesthetized but not dead. An aliquot of blood (500 μL) was mixed with an equal volume of 4% dextran (mw 500,000) and the erythrocytes were allowed to settle. A cytospin preparation was made from the resulting leukocyte rich fraction (top) and the cytospin was fixed and stained with Diff-Quick Stain kit (Dade Behring Inc, Newark, Del.). An aliquot of the leukocyte rich fraction was taken for total leukocyte count using flow cytometer (Guava EasyCyte Mini system). Differential leukocyte counts were obtained from the cytospin preparations. Blood eosinophil numbers were determined from the total leukocyte count and the percentage eosinophils.

Human Whole Blood CD11b Antagonist Assay (modified from Nicholson, et al. Pulmonary Pharmacology and Therapeutics: 20 (2007); 52-59). The potential CRTH2 antagonist activity of certain compounds was tested in human whole blood using an assay that tests the ability of the compounds to block the CD11b expression in eosinophils by 15-R-methyl-PGD2. A CRTH2 antagonist should block CD11b expression by subsequently added 15-Methyl-PGD2. Human whole blood (200 μL) was incubated at 37° C. for 10 minutes in the presence of various concentrations of test compounds before being challenged with the agonist 15R-Methyl-PGD2 (10 nM). Reactions were terminated by the addition of ice-cold PBS+0.5% BSA+2 mMEDTA (1 mL) and centrifugation (300×g for 5 minutes at 4° C.) Cells were then incubated at 4° C. for 10 min in the presence of human IgG. Cells were then incubated for 30-45 min with a mixture of PE-Cy5-labeled mouse anti-human CD16 (10 ul; BD Biosciences) and FITC-labeled mouse anti-human CD11b (10 μL; Beckman Coulter.) After rinsing (1 mL ice-cold PBS+0.5% BSA+2 mMEDTA), red blood cells were lysed by the addition of 1 mL ice-cold H₂O to the cell pellet for 30 sec-1 min immediately followed by the addition of 3.5% NaCl (300 μL.) Cells were then rinsed (2x−1 ml ice cold PBS+0.5% BSA+2 mMEDTA) and fixed in PBS containing 1% formaldehyde. The distribution of fluorescence intensities was measured by flow cytometry. Eosinophils were gated out on the basis of their granularity (high side scatter) and absence of CD-16. CD11b was then measured on this eosinophil population on the basis of fluorescence due to FITC.

DPBS CD11b Antagonist Assay. The potential CRTH2 antagonist activity of certain compounds was tested using a CD11b expression assay using essentially the method described by Monneret et al. (J Pharmacol Exp Ther 304:349-55, 2003). Briefly, polymorphonuclear cells (0.5 ml; 10⁶/ml cells) in PBS containing 0.9 mM CaCl₂ and 0.5 mM MgCl₂) were preincubated with various concentrations of test compounds at room temperature for 10 minutes before they were challenged with the agonist 15R-Methyl-PGD2 (10 nM). The incubations were terminated by the addition of ice-cold FACSFlow (BD Biosciences; Cat# 342003) and centrifugation (400 g for 5 minutes at 4° C.). The cells were then incubated for 30 minutes at 4° C. with a mixture of PE-labeled mouse anti-human VLA-4 (5 μl; BD Biosciences) and FITC-labeled mouse anti-human CD11b (10 μl; Beckman Coulter). The cells were then incubated with Optilyse C (0.25 ml; Beckman Coulter) for 15 minutes, centrifuged, and then fixed in PBS (0.4 ml; calcium and magnesium free) containing 1% formaldehyde. The distribution of fluorescence intensities among 60,000 cells was measured by flow cytometry. Eosinophils were gated out on the basis of their granularity (high side scatter) and labeling with VLA-4 (PE fluorescence). CD11b was then measured in the eosinophil region on the basis of fluorescence due to FITC. All data were corrected for the value obtained for the corresponding isotope control antibody.

Results of testing are shown below in Table 2.

TABLE 2

| | CD11b | | DK-PGD2 Induced |
|---|---|---|---|
| | Antagonist | CD11b Antagonist | Systemic |
| | DPBS | Human Whole Blood | Eosinophilia |
| Compound No. | IC₅₀(nM) | IC₅₀(nM) | ED₅₀ (mg/kg) |
| I-1 | A | B | B |
| I-2 | A | C | B |
| I-3 | A | B | |
| I-4 | A | B | A |
| I-5 | A | B | |
| I-6 | A | C | |
| I-7 | A | B | |
| I-8 | A | B | |
| I-9 | | | A |
| I-10 | A | C | |
| I-11 | A | C | A |
| I-12 | A | B | |
| I-13 | A | C | |
| I-14 | A | B | |
| I-15 | A | | |
| I-16 | B | | |
| I-17 | A | B | A |
| I-18 | A | | |
| I-19 | B | | A |
| I-20 | B | B | A |
| I-21 | B | C | A |
| I-22 | B | | A |
| I-23 | | | B |
| I-25 | C | | |
| I-26 | A | | |
| I-27 | B | | |
| I-34 | B | D | |
| I-35 | A | | |
| I-36 | A | B | |
| I-37 | A | | |
| I-38 | A | D | ND |
| I-39 | A | A | |
| I-40 | A | B | A |
| I-41 | C | D | |
| I-42 | C | D | |
| I-43 | A | B | A |
| I-44 | D | D | ND |
| I-45 | D | D | |
| I-48 | A | A | ND |
| I-49 | A | | B |
| I-50 | D | | |
| I-51 | C | | A |
| I-52 | A | | ND |
| I-53 | D | | |
| I-54 | C | | |
| I-55 | C | | A |
| I-56 | C | | |
| I-57 | B | | |
| I-58 | B | | |
| I-59 | B | | |
| I-60 | D | | |
| I-62 | A | | |
| I-66 | B | | |
| I-63 | C | C | |
| I-75 | D | | |
| I-77 | A | | |
| I-81 | A | | |
| I-82 | A | | |
| I-84 | D | | |
| I-95 | A | | |
| I-96 | A | | |

Activity for CD11b Antagonist Activity (DPBS or Human Whole Blood):
A: Less than 5 nM
B: Greater than or equal to 5 nM and less than 100 nM
C: Greater than or equal to 100 nM and less than 500 nM
D: Greater than 500 nM
Activity for Systemic Eosinophilia:
A: Less than 1 mg/kg
B: Greater than or equal to 1 mg/kg and less than 10 mg/kg
ND: Compound tested but ED₅₀ not determined by test

Empty cells indicate that the test was not done.

A number of embodiments have been described. Nevertheless, it will be understood that various modifications may be made without departing from the spirit and scope of the invention.

1. A compound of formula I
wherein
A is a 5, 6, 7, 8, 9 or 10-membered non-aromatic carbocycle; wherein A is optionally substituted with up to eight instances of R⁸;
L is chosen from
—O—,
—S(O)ₘ—,
—NR¹⁴— and
(C₁-C₃)alkylene, wherein, when said (C₁-C₃)alkylene is a C₂- or C₃-alkylene, one CH₂ is optionally replaced by —O—, —S(O)ₘ— or —NR¹⁴—, and wherein one or more substitutable carbon atoms of said (C₁-C₃)alkylene is optionally substituted with up to three instances of R¹¹;
X is chosen from a direct bond and (C₁-C₂)alkylene, wherein said (C₁-C₂)alkylene is optionally substituted with up to two instances of R¹²;
R¹ is chosen from (3-8-membered)carbocyclyl, (3-8-membered) heterocyclyl, —NR⁶(C₁-C₆)alkyl, —NR⁶(3-8-membered)carbocyclyl and —NR⁶(3-8-membered)heterocyclyl; wherein R¹ is optionally substituted with up to four instances of R⁹;
R² is chosen from hydrogen, halogen, (C₁-C₄)alkyl, (C₁-C₄)haloalkyl, and OH;
R³ is chosen from hydrogen, halogen, (C₁-C₄)alkyl and (C₁-C₄)haloalkyl;
R⁴ is chosen from hydrogen, halogen, (C₁-C₄)alkyl and (C₁-C₄)haloalkyl; or,
R³ and R⁴, taken together, form a (C₃-C₇)cycloalkyl ring;
R⁵ is chosen from C(O)OR⁷, C(O)N(R⁷)₂, C(O)NOR⁷ and C(O)NSR⁷;
R⁶ is chosen from hydrogen and (C₁-C₆)alkyl;
R⁷ is selected from hydrogen and (C₁-C₄)alkyl, wherein said (C₁-C₄)alkyl is optionally substituted with up to four instances of R¹⁶;
R⁸ in each occurrence is independently selected from halogen, (C₁-C₄)alkyl, (C₁-C₄)haloalkyl, (C₁-C₄)alkoxy, (C₁-C₄)haloalkoxy, CN, OH, oxo and N(R¹⁴)₂;
R⁹ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁴)₂;
R¹⁰ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;
R¹¹ in each occurrence is independently selected from halogen, (C₁-C₄)alkyl, (C₁-C₄)haloalkyl, (C₃-C₆)cycloalkyl, CN, OH, (C₁-C₄)alkoxy and (C₁-C₄)haloalkoxy;
R¹² in each occurrence is independently selected from halogen, (C₁-C₄)alkyl and (C₁-C₄)haloalkyl;
R¹⁴ is selected from hydrogen and (C₁-C₄)alkyl, wherein said (C₁-C₄)alkyl is optionally substituted with up to four instances of R¹⁰;
R¹⁵ is selected from hydrogen and (C₁-C₄)alkyl;
R¹⁶ in each occurrence is independently selected from halogen, (C₁-C₄)alkyl, (C₁-C₄)haloalkyl, (C₁-C₄)alkylcarbonyl, (C₁-C₄)alkoxycarbonyl, (3-8-membered)carbocyclyl, (3-8-membered) heterocyclyl, CN, OH and N(R¹⁵)₂;
m is zero, one or two; and
n is zero, one or two.

2. The compound of claim 1, wherein R¹⁶ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;

3. The compound of claim 1 or 2 wherein A is a fused cycloheptyl ring optionally substituted with up to eight instances of R⁸, independently selected, wherein said compound has the formula:
and p is zero or an integer from 1 to 8.

4. The compound of claim 1 or 2 wherein A is a fused cyclohexyl ring optionally substituted with up to eight instances of R⁸, independently selected, wherein said compound has the formula:
and p is zero or an integer from 1 to 8.

5. The compound of claim 4 wherein two R⁸ are each a methyl residue attached to the same ring carbon.

6. The compound of claim 5 of formula
wherein t is zero or an integer from 1 to 4.

7. The compound of claim 6, wherein t is zero.

8. The compound of claim 4, wherein the fused cyclohexyl ring is substituted with up to eight instances of R⁸ independently selected from the group consisting of halogen, (C1-C4)alkyl and oxo.

9. The compound of claim 8 having one of the following three formulae:
wherein t is 0 or an integer of from 1 to 6.

10. The compound of claim 9, wherein the up to six instances of R⁸ are independently selected from the group consisting of fluoro and methyl.

11. The compound of claim 10, wherein t is 2 or 4.

12. The compound of claim 4 having the formula:
and p is zero or an integer from 1 to 4.

13. The compound of claim 12 wherein p is zero or R⁸ is an oxo.

14. The compound of claim 1 or 2 wherein A is a fused cyclopentyl ring optionally substituted with up to six instances of R⁸, independently selected, wherein said compound has the formula:
and q is zero or an integer from 1 to 6.

15. The compound of claim 1, wherein X is chosen from a direct bond, —CH₂— and —CH₂CH₂—.

16. The compound of claim 15, wherein X is a direct bond.

17. The compound of claim 1, wherein R² is chosen from hydrogen, fluoro, methyl, ethyl and trifluoromethyl.

18. The compound of claim 17 wherein R² is methyl.

19. The compound of claim 1, wherein R³ and R⁴ are taken together to form a cyclopropyl ring.

20. The compound of claim 1, wherein R³ and R⁴ are each independently selected from hydrogen and methyl, wherein said methyl is optionally substituted with 1-3 instances of halogen.

21. The compound of claim 20, wherein said halogen is fluoro.

22. The compound of claim 21, wherein R³ and R⁴ are each hydrogen.

23. The compound of claim 1, wherein L is —CH₂—, —O— or —S(O)ₘ—.

24. The compound of claim 23 wherein L is —CH₂—.

25. The compound of claim 1, wherein L is —S(O)ₘ— and m is 2.

26. The compound according to claim 1, wherein X is a direct bond and R⁵ is C(O)OR⁷, C(O)N(R⁷)₂, C(O)NHOR⁷ or C(O)NHSR⁷, wherein R⁷ is H or (C₁₋₄)alkyl.

27. The compound of claim 22, wherein R⁵ is C(O)OH or C(O)O(C₁₋₄)alkyl.

28. The compound of claim 27, wherein R⁵ is C(O)OH.

29. The compound of claim 1, wherein R¹ is a non-aromatic 3-8 membered heterocycle, phenyl, or a non-aromatic 3-8 membered carbocycle, wherein R¹ is optionally substituted with up to four instances of R⁹.

30. The compound of claim 29, wherein R¹ is phenyl or a non-aromatic 3-8 membered carbocycle, wherein R¹ is optionally substituted with up to four instances of R⁹.

31. The compound of claim 30, wherein R¹ is a non-aromatic 3-8 membered heterocycle, wherein R¹ is optionally substituted with up to four instances of R⁹.

32. The compound of claim 31, wherein R¹ is a non-aromatic 5-7 membered heterocycle, wherein R¹ is optionally substituted with one to three instances of R⁹.

33. The compound of claim 32 wherein R¹ is an N-attached pyrrolidine, piperidine, piperazine, azepine or morpholine, optionally substituted with one to three instances of R⁹, wherein each R⁹ is independently selected from (C₁-C₄)alkyl and (C₁-C₄)haloalkyl.

34. The compound of claim 33, wherein R¹ is an N-attached piperazine of formula
wherein R¹³ is chosen from hydrogen, (C₁-C₄)alkyl, (C₁-C₄)alkylcarbonyl and (C₁-C₄)alkoxycarbonyl and u is zero, one or two.

35. The compound of claim 33 wherein R¹ is an N-attached morpholine of formula
wherein u is zero, one or two.

36. The compound of claim 1, wherein —S(O)ₙR¹ is attached para or ortho to L on the phenyl ring.

37. The compound of claim 36, wherein said compound has one of the following formulae:
wherein p is zero or an integer from 1 to 3.

38. The compound of claim 37, wherein said compound has the following formulae:
wherein p is zero or one.

39. The compound of claim 37 wherein —S(O)ₙR¹ is attached para or ortho to L on the phenyl ring.

40. The compound of claim 39, wherein X is a direct bond and R⁵ is C(O)OH or C(O)O(C₁₋₄)alkyl.

41. The compound of claim 1, wherein R¹ is —NR⁶(C₁-C₆)alkyl and R⁶ is hydrogen or methyl.

42. The compound of claim 1 or 2 wherein
A is chosen from a cyclopentyl ring, a cycloheptyl ring and a dimethylcyclohexyl ring;
R² is methyl;
R³ and R⁴ are hydrogen;
L is —CH₂—;
n is 2; and
R¹ is chosen from
(a) pyrrolidinyl, piperidinyl, azepinyl or morpholinyl;
(b) piperazine-1-yl substituted at 4 with (C₁-C₄)alkylcarbonyl or (C₁-C₄)alkoxycarbonyl;
(c) —NR⁶(C₁-C₆)alkyl wherein R⁶ is hydrogen or methyl; and
(d) phenyl and substituted phenyl.

43. The compound of claim 1 which has the formula of II
in which
A is chosen from a cyclopentyl ring, a cycloheptyl ring, a cyclohexyl ring, and a dimethylcyclohexyl ring;
R⁷ is hydrogen or (C₁₋₄) alkyl;
L is —CH₂— or —S(O)ₘ—;
m is 0 or 2;
R⁸ is oxo;
t is 0, 1 or 2; and
R¹ is chosen from
(a) pyrrolidinyl, piperidinyl, azepinyl or morpholinyl;
(b) piperazine-1-yl substituted at the 4 position with (C₁-C₄)alkylcarbonyl or (C₁-C₄)alkoxycarbonyl;
(c) —NR⁶(C₁-C₆)alkyl wherein R⁶ is hydrogen or methyl; and
(d) phenyl and substituted phenyl.

44. The compound of claim 43, wherein A is a cyclohexyl ring or a dimethylcyclohexyl ring.

45. The compound of claim 44, wherein the compound has the formula of II′ or II″:

46. The compound of claim 1, wherein L is —CH₂—.

47. The compound of claim 1, wherein t is one and R⁸ is oxo.

48. The compound of claim 1, wherein the compound has the formula of III:

49. The compound of claim 1, wherein R¹ is chosen from pyrrolidine, piperidinyl, azepinyl, morpholinyl or phenyl.

50. The compound according to claim 49, wherein R¹ is chosen from pyrrolidine or morpholine.

51. The compound according to claim 1, wherein —(SO)₂R¹ is attached para or ortho to L on the phenyl ring.

52. A compound selected from the compounds listed in Table 1.

53. A composition comprising a pharmaceutically acceptable carrier and a compound according to claim 1.

54. A method for treating a patient suffering from a disease or disorder involving the CRTH2 receptor comprising administering to said patient a therapeutically effective amount of a compound according to claim 1 or a composition comprising said compound.

55. The method according to claim 54, wherein said disease or disorder is asthma, atopic dermatitis, allergic rhinitis, allergy, Grave's Disease, acute rhinitis, hatrophic rhinitis or chronic rhinitis, rhinitis caseosa, hypertrophic rhinitis, rhinitis purulenta, rhinitis sicca, rhinitis medicamentosa, membranous rhinitis, croupous rhinitis, fibrinous rhinitis, pseudomembranous rhinitis, scrofulous rhinitis, perennial allergic rhinitis, seasonal rhinitis, rhinitis nervosa, vasomotor rhinitis, antitussive activity, bronchial asthma, allergic asthma, intrinsic asthma, extrinsic asthma, dust asthma, chronic asthma, inveterate asthma, late asthma, airway hyper-responsiveness, bronchitis, chronic bronchitis, eosinophilic bronchitis, chronic inflammatory diseases of the lung which result in interstitial fibrosis, interstitial lung diseases (ILD), idiopathic pulmonary fibrosis, ILD associated with rheumatoid arthritis, scleroderma lung disease, chronic obstructive pulmonary disease (COPD), chronic sinusitis, conjunctivitis, allergic conjunctivitis, cystic fibrosis, fanner's lung, fibroid lung, hypersensitivity lung disease, hypersensitivity pneumonitis, idiopathic interstitial pneumonia, nasal congestion, nasal polyposis, otitis media, chronic cough associated with inflammation, systemic anaphylaxis, hypersensitivity responses, drug allergies, insect sting allergies, food related allergies, food-related allergies with symptoms of migraine, rhinitis or eczema, arthritis, rheumatic arthritis, infectious arthritis, autoimmune arthritis, seronegative arthritis, spondyloarthropathy, ankylosing spondylitis, psoriatic arthritis, Reiter's disease, osteoarthritis, systemic sclerosis, psoriasis, atopical dermatitis, contact dermatitis, seborrheic dermatitis, cutaneous eosinophilias, chronic skin ulcers, cutaneous lupus erythematosus, contact hypersensitivity, allergic contact dermatitis, eosinophilic folliculitis, Coeliac disease, cholecystitis, Crohn's disease, enteritis, eosinophilic gastroenteritis, eosinophilic esophagitis, enteropathy associated with seronegative arthropathies, gastritis, inflammatory bowel disease, irritable bowel disease, acute and chronic allograft rejection following solid organ transplant, chronic graft versus host disease, skin graft rejection, bone marrow transplant rejection, inflammation, hyperalgesia, allodynia, neuropathic pain, lupus erythematosus; systemic lupus, erythematosus; Hashimoto's thyroiditis, Grave's disease, type I diabetes, eosinophilia fasciitis, hyper IgE syndrome, idiopathic thrombocytopenia pupura; post-operative adhesions, ischemic/reperfusion injury in the heart, brain, peripheral limb hepatitis, mastocytosis, mastitis, vaginitis, vasculitis, myositis, basophilic leukemia, basophilic leukocytosis, or Churg-Strauss syndrome.

56. The method according to claim 55 for treating asthma or preventing an asthma attack.

57. The method according to claim 55 for treating allergic rhinitis.

58. The method according to claim 55 for treating Chronic Obstructive Pulmonary Disease.

59. The method according to claim 54 for treating neuropathic pain.

60. The method according to claim 55 for treating atopic dermatitis.

61. The method according to claim 55 for treating allergic conjunctivitis.

62. The method according to claim 55 for treating gastrointestinal tract related diseases and disorders selected from Crohn's disease, eosinophilic gastroenteritis, eosinophilic esophagitis, inflammatory bowel disease or irritable bowel disease.
